(19) United States
(12) Patent Application Publication
(10) Pub. No.: US 2016/0229839 A1
(43) Pub. Date: Aug. 11, 2016

(54) Factor XIa Inhibitors

(71) Applicants: Eric Mertz, Somerset, NJ (US); Scott D. Edmondson, Clark, NJ (US); Ning Shao, Watchung, NJ (US); Santhosh Neelamkavil, Edison, NJ (US); Corey Poker, Carteret, NJ (US); Zahid Hussain, Dayton, NJ (US); Zhuyan Guo, Scotch Plains, NJ (US); Nancy Jo Kevin, East Brunswick, NJ (US); Yi Zang, Princeton Junction, NJ (US); Jiafang He, Dayton, NJ (US)

(72) Inventors: Eric Mertz, Somerset, NJ (US); Scott D. Edmondson, Clark, NJ (US); Ning Shao, Watchung, NJ (US); Santhosh Neelamkavil, Edison, NJ (US); Corey Poker, Carteret, NJ (US); Zahid Hussain, Dayton, NJ (US); Zhuyan Guo, Scotch Plains, NJ (US); Nancy Jo Kevin, East Brunswick, NJ (US); Yi Zang, Princeton Junction, NJ (US); Jiafang He, Dayton, NJ (US)

(73) Assignee: Merck Sharp & Dohme Corp., Rahway, NJ (US)

(21) Appl. No.: 15/023,833

(22) PCT Filed: Sep. 23, 2014
(86) PCT No.: PCT/US14/56873

§ 371 Date: Mar. 22, 2016

Related U.S. Application Data
(60) Provisional application No. 61/883,510, filed on Sep. 27, 2013.

(51) Int. Cl. C07D 401/14; A01N 1/02; C07D 401/06; C07D 401/10; C07D 211/60

(52) U.S. Cl. CPC C07D 401/14; C07D 401/10; C07D 211/60; C07D 401/06; A01N 1/0226

(57) ABSTRACT

The present invention provides a compound of Formula (I)

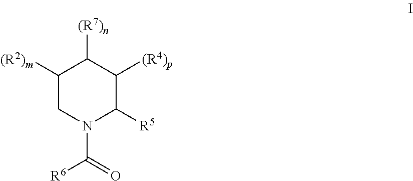

and pharmaceutical compositions comprising one or more said compounds, and methods for using said compounds for treating or preventing thromboses, embolisms, hypercoagulability or fibrotic changes. The compounds are selective Factor XIa inhibitors or dual inhibitors of Factor XIa and plasma kallikrein.

### BACKGROUND OF THE INVENTION

Factor XIa is a plasma serine protease involved in the regulation of blood coagulation. While blood coagulation is a necessary and important part of the regulation of an organism's homeostasis, abnormal blood coagulation can also have deleterious effects. For instance, thrombosis is the formation or presence of a blood clot inside a blood vessel or cavity of the heart. Such a blood clot can lodge in a blood vessel blocking circulation and inducing a heart attack or stroke. Thromboembolic disorders are the largest cause of mortality and disability in the industrialized world.

Blood clotting is a process of control of the blood stream essential for the survival of mammals. The process of clotting, and the subsequent dissolution of the clot after wound healing has taken place, commence after vascular damage, and can be divided into four phases. The first phase, vasoconstriction or vasocontraction, can cause a decrease in blood loss in the damaged area. In the next phase, platelet activation by thrombin, platelets attach to the site of the vessel wall damage and form a platelet aggregate. In the third phase, formation of clotting complexes leads to massive formation of thrombin, which converts soluble fibrinogen to fibrin by cleavage of two small peptides. In the fourth phase, after wound healing, the thrombus is dissolved by the action of the key enzyme of the endogenous fibrinolysis system, plasmin.

Two alternative pathways can lead to the formation of a fibrin clot, the intrinsic and the extrinsic pathway. These pathways are initiated by different mechanisms, but in the later phase they converge to give a common final path of the clotting cascade. In this final path of clotting, clotting factor X is activated. The activated factor X is responsible for the formation of thrombin from the inactive precursor prothrombin circulating in the blood. The formation of a thrombus on the bottom of a vessel wall abnormality without a wound is the result of the intrinsic pathway. Fibrin clot formation as a response to tissue damage or an injury is the result of the extrinsic pathway. Both pathways comprise a relatively large number of proteins, which are known as clotting factors. The intrinsic pathway requires the clotting factors V, VIII, IX, X, XI and XII and also prekallikrein, high molecular weight kininogen, calcium ions and phospholipids from platelets. The activation of factor XIa is a central point of intersection between the two pathways of activation of clotting. Factor XIa has an important role in blood clotting.

Coagulation is initiated when blood is exposed to artificial surfaces (e.g., during hemodialysis, “on-pump” cardiovascular surgery, vessel grafts, bacterial sepsis), on cell surfaces, cellular receptors, cell debris, DNA, RNA, and extracellular matrices. This process is also termed contact activation. Surface absorption of factor XII leads to a conformational change in the factor XII molecule, thereby facilitating activation to proteolytic active factor XII molecules (factor XIIa and factor XIIf). Factor XIIa (or XIIf) has a number of target proteins, including plasma prekallikrein and factor XI. Active plasma kallikrein further activates factor XII, leading to an amplification of contact activation. Alternatively, the serine protease prolylcarboxylpeptidase can activate plasma kallikrein complexed with high molecular weight kininogen in a multiprotein complex formed on the surface of cells and matrices (Shariat-Madar et al., Blood, 108:192-199 (2006)). Contact activation is a surface mediated process responsible in part for the regulation of thrombosis and inflammation, and is mediated, at least in part, by fibrinolytic-, complement-, kininogen/kinin-, and other humoral and cellular pathways (for review, Coleman, R., “Contact ActivationPathway”, Hemostasis and Thrombosis, pp. 103-122, Lippincott Williams & Wilkins (2001); Schmaier, A. H., “Contact Activation”, Thrombosis and Hemorrhage, pp. 105-128 (1998)). The biological relevance of the contact activation system for thromboembolic 5 diseases is supported by the phenotype of factor XII deficient mice. More specifically, factor XII deficient mice were protected from thrombotic vascular occlusion in several thrombosis models as well as stroke models and the phenotype of the XII deficient mice was identical to XI deficient mice (Renne et al., J Exp. Med., 202:271-281 (2005); Kleinschmitz et al., J Exp. Med., 203:513-518 (2006)). The fact that factor XI is downstream from factor XIIa, combined with the identical phenotype of the XII and XI deficient mice suggest that the contact activation system could play a major role in factor XI activation in vivo. Plasma kallikrein is a zymogen of a trypsin-like serine protease and is present in plasma. The gene structure is similar to that of factor XI. Overall, the amino acid sequence of plasma kallikrein has 58% homology to factor XI. Proteolytic activation by factor XIIa at an internal I 389-R390 bond yields a heavy chain (371 amino acids) and a light chain (248 amino acids). The active site of plasma kallikrein is contained in the light chain. The light chain of plasma kallikrein reacts with protease 15 inhibitors, including alpha 2 macroglobulin and Cl-inhibitor. Interestingly, heparin significantly accelerates the inhibition of plasma kallikrein by antithrombin III in the presence of high molecular weight kininogen (HMWK). In blood, the majority of plasma kallikrein circulates in complex with HMWK. Plasma kallikrein cleaves HMWK to liberate bradykinin. Bradykinin release results in increase of vascular permeability and vasodilation (for review, Coleman, R., “Contact Activation Pathway”, Hemostasis and Thrombosis, pp. 103-122, Lippincott Williams & Wilkins (2001); Schmaier A. H., “Contact Activation”, Thrombosis and Hemorrhage, pp. 105-128 (1998)).

Factor XIa inhibitor compounds are described in WO2013022814, WO 2013022814, WO 2013022818, WO 2013055984, WO2013056034, WO2013056060, WO2013118805. WO2013093484, WO2002042273, WO2002037937, WO2002060894, WO2003015715, WO2004002405, US20040180855, WO2004080971, WO2004094372, US20050228000, US20050282805, WO2005123680, US20090036438, US20120088758, US20060074103, WO2006062972, WO2006076246, US20060154915, US20090062287, US20060183771, WO2007070818, WO2007070816, WO2007070826, WO2008076805, WO2008157162, WO2009114677, WO2011100402, and WO2011100401.

### SUMMARY OF THE INVENTION

The present invention relates to compounds of Formula I:

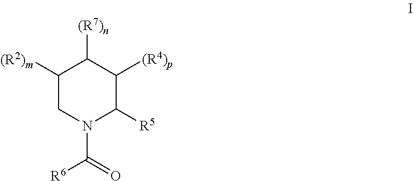

or pharmaceutically acceptable salts thereof. The compounds of Formula I are selective Factor XIa inhibitors or dual inhibitors of Factor XIa and plasma kallikrein, and as such may be useful in the treatment, inhibition or amelioration of one or more disease states that could benefit from inhibition of Factor XIa or plasma kallikrein, including thromboses, embolisms, hypercoagulability or fibrotic changes. The compounds of this invention could further be used in combination with other therapeutically effective agents, including but not limited to, other drugs useful for the treatment of thromboses, embolisms, hypercoagulability or fibrotic changes. The invention furthermore relates to processes for preparing compounds of Formula I, and pharmaceutical compositions which comprise compounds of Formula I and pharmaceutically acceptable salts thereof.

### DETAILED DESCRIPTION OF THE INVENTION

The invention includes compounds of Formula I

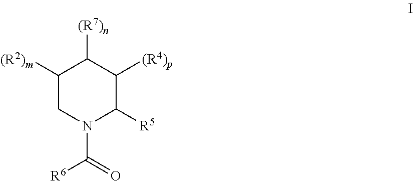

or a pharmaceutically acceptable salt thereof, wherein
m is 0 or 1, n is 0 or 1, and p is 0 or 1, provided that m+n+p=1;

R² and R⁷ are independently selected from aryl, heterocyclyl, and C₃₋₆cycloalkyl;

### R⁶ is

1) —CH═CH-aryl, wherein aryl is mono or disubstituted with a substituent independently selected from the group consisting of halogen and tetrazole,

2) —CH₂CH₂-aryl, wherein aryl is mono or disubstituted with a substituent independently selected from the group consisting of halogen and tetrazole,

3) 4-7 membered monocyclic heterocyclyl having one or two heteroatoms independently selected from N, O and S, which is unsubstituted or substituted at the nitrogen atom with —C(NH)NH₂,

4) 5-7 membered monocyclic cycloalkyl or aryl, wherein cycloalkyl or aryl is unsubstituted or substituted with one or two of —CH₂NH₂, NH₂, C(CH₃)₂NH₂, C₁₋₆ alkyl, or C₃₋₈ cycloalkyl,

5) 7-9 membered bicyclic aryl, which is unsubstituted or substituted with NH₂, or

6)

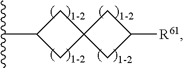

wherein R⁶¹ is —(CH₂)₀₋₁—NH₂;

### R⁴ is

1) —CH₂OR⁴¹, wherein R⁴¹ is hydrogen or —Si(C₁₋₆ alkyl)(C₁₋₆ alkyl)(C₁₋₆ alkyl),

2) aryl, or

3) 3-7 membered monocyclic cycloalkyl,
wherein aryl is unsubstituted or substituted with one or two substituents independently selected from CF₃, halogen, NH₂, OCF₃, C(O)NH₂, C₁₋₆alkyl or

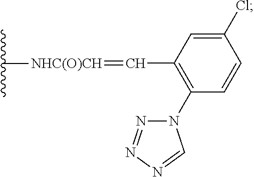

### R⁵ is

1) —C(O)NHR⁵¹ wherein
R⁵¹ is
a) 3-7 membered monocyclic aryl or cycloalkyl,
b) 8 membered bicyclic cycloalkyl, or
c) 9 membered bicyclic heteroaryl,
wherein aryl or cycloalkyl is unsubstituted or substituted with one or two substituents independently selected from C(O)OC(C₁₋₆ alkyl)(C₁₋₆ alkyl)(C₁₋₆ alkyl) or C(O)OH, and wherein heteroaryl is unsubstituted or substituted with methyl or CHOH,

2)

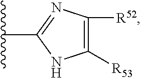

wherein
R⁵² is
a) 3-7 membered monocyclic aryl,
b) 6 membered heteroaryl containing 1 nitrogen atom, or
c) 9 membered bicyclic heteroaryl containing 1 or 2 heteroatoms, selected from N and O
wherein aryl is unsubstituted or substituted with with one or two substituents independently selected from CN, halogen, OC₁₋₆ alkyl, SO₂C₁₋₆ alkyl, CF₃, C(O)OC₁₋₆alkyl, NH₂, NHC(O)OC₁₋₆ alkyl, NHC(O)C₁₋₆ alkyl, C(O)OC(CH₃)₂, C(O)OH, PO₃H₂, or PO₃(C₁₋₆ alkyl)₂, wherein heteroaryl is unsubstituted or substituted with methyl or NH₂, and wherein bicyclic heteroaryl is unbsubstituted or substituted with a ═O, and
R⁵³ is hydrogen, halogen or C₁₋₆ alkyl, and

3)

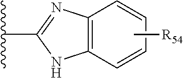

wherein

R⁵⁴ is hydrogen or halogen.

In another embodiment of the invention, m is 0, n is 0, p is 1, and R⁴ is

1) —CH₂OR⁴¹, wherein R⁴¹ is hydrogen or —Si(C₁₋₆ alkyl)(C₁₋₆ alkyl)(C₁₋₆ alkyl),

2) aryl, or

3) 5-7 membered monocyclic cycloalkyl,

wherein aryl is unsubstituted or substituted with one or two substituents independently selected from CF₃, halogen, NH₂, OCF₃, C(O)NH₂, C₁₋₆alkyl or

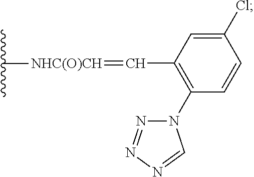

In another embodiment of the invention, R⁶ is

1) —CH═CH-aryl, wherein aryl is substituted with chloro and substituted with tetrazole,

2) —CH₂CH₂-aryl, wherein aryl is mono or disubstituted with a substituent independently selected from the group consisting of halogen and tetrazole,

3) 4-7 membered heterocyclyl having one N atom, wherein heterocyclyl is unsubstituted or substituted at the nitrogen atom with —C(NH)NH₂,

4) 5-7 membered monocyclic aryl, wherein aryl is unsubstituted or substituted with one or two substituents independently selected from —CH₂NH₂, NH₂, C(CH₃)₂NH₂, C(CH₃)₃, or cyclopropyl or

5) 7-9 membered bicyclic aryl, wherein bicyclic aryl is unsubstituted or substituted with NH₂.

In another embodiment of the invention, m is 0, n is 0, p is 1, and R⁶ is

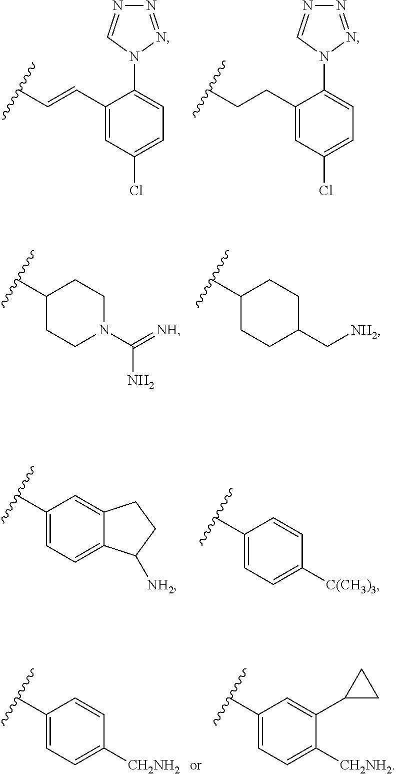

In another embodiment of the invention, m is 0, n is 0, p is 1 and R⁴ is

1) —CH₂OR⁴¹, wherein R⁴¹ is hydrogen or —Si(CH₃)₂(C(CH₃)₃),

2) phenyl, or

3) 5-7 membered monocyclic cycloalkyl, wherein phenyl is unsubstituted or substituted with with one or two substituents independently selected from CF₃, F, NH₂, OCF₃, C(O)NH₂, methyl, or

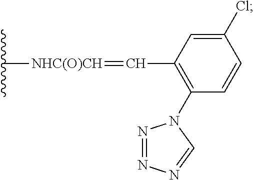

In another embodiment of the invention, m is 0, n is 0, p is 1, and p is 1 and R⁴ is

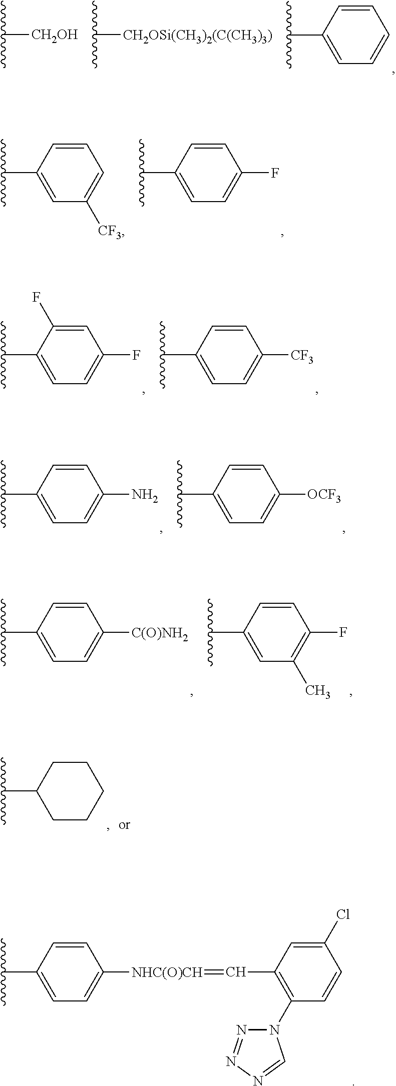

In another embodiment of the invention, m is 0, n is 0, p is 1, R⁵ is

1) —C(O)NHR⁵¹, wherein R⁵¹ is

a) 5-7 membered monocyclic aryl or cyclalkyl,

b) 8 membered bicyclic cycloalkyl, or

c) 9 membered bicyclic unsaturated heterocyclyl,

wherein alkyl or cycloalkyl is unsubstituted or substituted with one or two substituents independently selected from C(O)OC(CH₃)₂ or C(O)OH, and wherein heterocyclyl is unsubstituted or substituted with methyl or CHOH,

2)

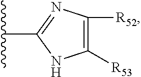

wherein

R⁵² is
a) 5-7 membered monocyclic aryl,
b) 6 membered heterocyclyl containing 1 nitrogen atom, or
c) 9 membered bicyclic heteroaryl containing 1 or 2 heteroatoms selected from N and O,
wherein aryl is unsubstituted or substituted with one or two substituents independently selected from CN, F, Cl, Br, OCH₃, SO₂CH₃, CF₃, C(O)OCH₃, NH₂, NHC(O)OCH, NHC(O)CH₂CH₂CH₃, C(O)OC(CH)₂, or C(O)OH, wherein heterocyclyl is unsubstituted or substituted with methyl or NH₂, and wherein bicyclic heteroaryl is unsubstituted or substituted with ═O, and

R⁵³ is hydrogen, Cl or methyl, or

3)

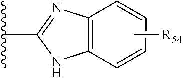

wherein R⁴ is hydrogen or F.

In another embodiment of the invention, m is 0, n is 0, p is 1, R⁵ is

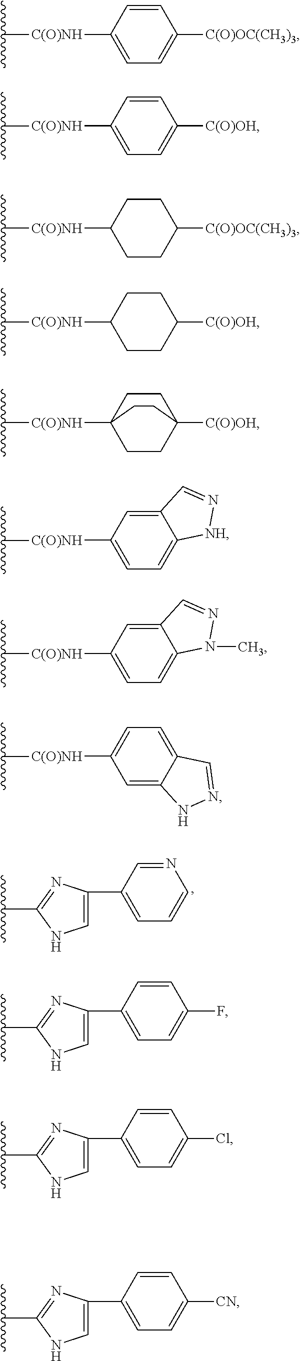

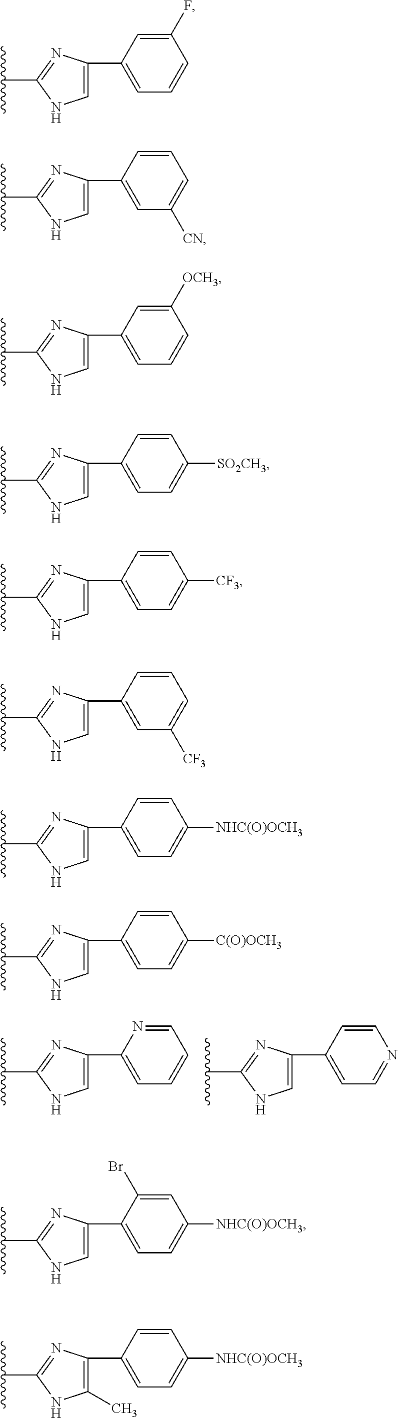

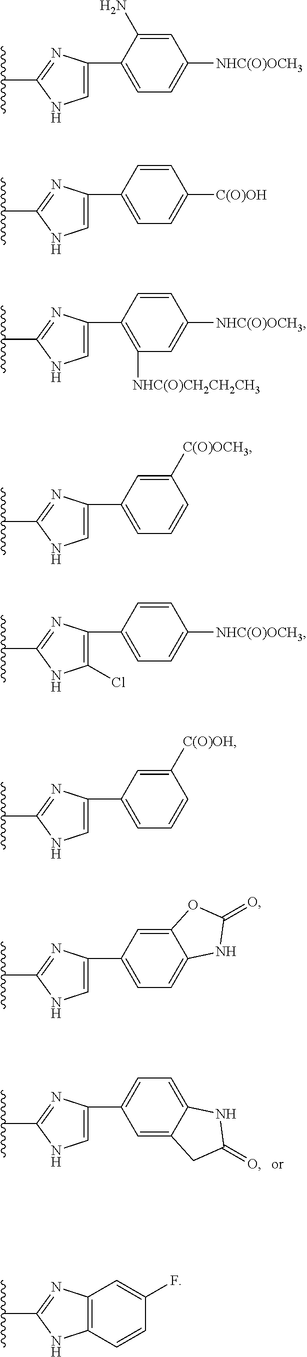

In another embodiment of the invention, the compound is
1-1: (E)-tert-butyl 4-(1-(3-(5-chloro-2-(1H-tetrazol-1-yl)phenyl)acryloyl)-3-phenylpiperidine-2-carboxamido)benzoate,
1-2: (E)-1-(2-(3-(2-((4-carboxyphenyl)carbamoyl)-3-phenylpiperidin-1-yl)-3-oxoprop-1-en-1-yl)-4-chlorophenyl)-1H-tetrazol-2-ium 2,2,2-trifluoroacetate,
1-3: (E)-tert-butyl 4-(1-(3-(5-chloro-2-(1H-tetrazol-1-yl)phenyl)acryloyl)-3-cyclohexylpiperidine-2-carboxamido)benzoate,
1-4: (E)-tert-butyl 4-(1-(3-(5-chloro-2-(1H-tetrazol-1-yl)phenyl)acryloyl)-3-cyclohexylpiperidine-2-carboxamido)cyclohexanecarboxylate,
1-5: (E)-1-(2-(3-(2-((4-carboxyphenyl)carbamoyl)-3-cyclohexylpiperidin-1-yl)-3-oxoprop-1-en-1-yl)-4-chlorophenyl)-1H-tetrazol-2-ium 2,2,2-trifluoroacetate,
1-6: (E)-1-(2-(3-(2-((4-carboxyhexyl)carbamoyl)-3-cyclohexylpiperidin-1-yl)-3-oxoprop-1-en-1-yl)-4-chlorophenyl)-1H-tetrazol-2-ium 2,2,2-trifluoroacetate,
1-7: (E)-3-(5-chloro-2-(1H-tetrazol-1-yl)phenyl)-1-(3-phenyl-2-(4-(pyridin-3-yl)-1H-imidazol-2-yl)piperidin-1-yl)prop-2-en-1-one,
1-8: 4-(1-(1-carbamimidoylpiperidine-4-carbonyl)-3-phenylpiperidine-2-carboxamido)benzoic acid,
1-9: 4-(1-(3-(5-chloro-2-(1H-tetrazol-1-yl)phenyl)propanoyl)-3-phenylpiperidine-2-carboxamido)benzoic acid,
1-10: (E)-4-(1-(3-(5-chloro-2-(H-tetrazol-1-yl)phenyl)acryloyl)-3-(3-fluorophenyl)piperidine-2-carboxamido)benzoic acid,
1-11: (E)-3-(5-chloro-2-(1H-tetrazol-1-yl)phenyl)-1-(3-phenyl-2-(4-(pyridin-3-yl)-1H-imidazol-2-yl)piperidin-1-yl)prop-2-en-1-one (2R,3R)
1-12: (E)-3-(5-chloro-2-(1H-tetrazol-1-yl)phenyl)-1-(3-phenyl-2-(4-(pyridin-3-yl)-1H-imidazol-2-yl)piperidin-1-yl)prop-2-en-1-one (2S,3S),
1-13: (E)-3-(5-chloro-2-(1H-tetrazol-1-yl)phenyl)-1-(2-(4-(4-fluorophenyl)-1H-imidazol-2-yl)-3-phenylpiperidin-1-yl)prop-2-en-1-one,
1-14: 4-(1-(1-amino-2,3-dihydro-1H-indene-5-carbonyl)-3-phenylpiperidine-2-carboxamido)benzoic acid, rac cis,
1-15: 4-(1-(4-(2-aminopropan-2-yl)benzoyl)-3-phenylpiperidine-2-carboxamido)benzoic acid,
1-16: 4-(1-((S)-1-amino-2,3-dihydro-1H-indene-5-carbonyl)-3-phenylpiperidine-2-carboxamido)benzoic acid, cis,
1-17: ammonium (E)-4-(1-(3-(5-chloro-2-(1H-tetrazol-1-yl)phenyl)acryloyl)-3-(3-(trifluoromethyl)phenyl)piperidine-2-carboxamido)benzoate,
1-18: ammonium (E)-4-(1-(3-(5-chloro-2-(1H-tetrazol-1-yl)phenyl)acryloyl)-3-(4-fluorophenyl)piperidine-2-carboxamido)benzoate,
1-19: ammonium (E)-4-(1-(3-(5-chloro-2-(1H-tetrazol-1-yl)phenyl)acryloyl)-3-(2,4-difluorophenyl)piperidine-2-carboxamido)cyclohexanecarboxylate,
1-20: (4-(2-((4-carboxyphenyl)carbamoyl)-3-phenylpiperidine-1-carbonyl)phenyl)methanaminium 2,2,2-trifluoroacetate,
1-21: (E)-3-(5-chloro-2-(1H-tetrazol-1-yl)phenyl)-1-(2-(4-(4-chlorophenyl)-1H-imidazol-2-yl)-3-phenylpiperidin-1-yl)prop-2-en-1-one,
1-22: (E)-4-(2-(1-(3-(5-chloro-2-(1H-tetrazol-1-yl)phenyl)acryloyl)-3-phenylpiperidin-2-yl)-1H-imidazol-4-yl)benzonitrile,
1-23: 3-(5-chloro-2-(1H-tetrazol-1-yl)phenyl)-1-(2-(4-(4-chlorophenyl)-1H-imidazol-2-yl)-3-phenylpiperidin-1-yl)propan-1-one,
1-24: 4-(2-(1-(3-(5-chloro-2-(1H-tetrazol-1-yl)phenyl)propanoyl)-3-phenylpiperidin-2-yl)-1H-imidazol-4-yl)benzonitrile,
1-25: (E)-4-(1-(3-(5-chloro-2-(1H-tetrazol-1-yl)phenyl)acryloyl)-3-(3-(trifluoromethyl)phenyl)piperidine-2-carboxamido)benzoic acid,
1-26: (E)-4-(1-(3-(5-chloro-2-(1H-tetrazol-1-yl)phenyl)acryloyl)-3-(2,4-difluorophenyl)piperidine-2-carboxamido)benzoic acid,
1-27: (E)-3-(2-(1-(3-(5-chloro-2-(1H-tetrazol-1-yl)phenyl)acryloyl)-3-phenylpiperidin-2-yl)-1H-imidazol-4-yl)benzonitrile,
1-28: (E)-3-(5-chloro-2-(1H-tetrazol-1-yl)phenyl)-1-(2-(4-(3-methoxyphenyl)-1H-imidazol-2-yl)-3-phenylpiperidin-1-yl)prop-2-en-1-one,
1-29: (E)-3-(5-chloro-2-(1H-tetrazol-1-yl)phenyl)-1-(2-(4-(4-(methyl sulfonyl)phenyl)-1H-imidazol-2-yl)-3-phenyl piperidin-1-yl)prop-2-en-1-one,
1-30: (E)-3-(5-chloro-2-(1H-tetrazol-1-yl)phenyl)-1-(3-phenyl-2-(4-(4-(trifluoromethyl)phenyl)-1H-imidazol-2-yl)piperidin-1-yl)prop-2-en-1-one.
1-31: (E)-3-(5-chloro-2-(1H-tetrazol-1-yl)phenyl)-1-(3-phenyl-2-(4-(3-(tifluoromethyl)phenyl)-1H-imidazol-2-yl)piperidin-1-yl)prop-2-en-1-one,
1-32: (E)-4-(1-(3-(5-chloro-2-(1H-tetrazol-1-yl)phenyl)acryloyl)-3-(4-(trifluoromethyl)phenyl)piperidine-2-carboxamido)benzoic acid,
1-33:(E)-4-(3-(4-aminophenyl)-1-(3-(5-chloro-2-(1H-tetrazol-1-yl)phenyl)acryloyl)piperidine-2-carboxamido)benzoic,
1-34: 4-(3-(4-((E)-3-(5-chloro-2-(1H-tetrazol-1-yl)phenyl)acrylamido)phenyl)-1-((E)-3-(5-chloro-2-(1H-tetrazol-1-yl)phenyl)acryloyl)piperidine-2-carboxamido)benzoic acid,
1-35: (E)-4-(1-(3-(5-chloro-2-(1H-tetrazol-1-yl)phenyl)acryloyl)-3-(4-(tifluoromethoxy)phenyl)piperidine-2-carboxamido)benzoic acid,
1-36: (E)-4-(3-(4-carbamoylphenyl)-1-(3-(5-chloro-2-(1H-tetrazol-1-yl)phenyl)acryloyl)piperidine-2-carboxamido)benzoic acid,
1-37: (E)-4-(1-(3-(5-chloro-2-(1H-tetrazol-1-yl)phenyl)acryloyl)-3-(4-fluoro-3-methylphenyl)piperidine-2-carboxamido)benzoic acid,
1-38: (E)-methyl (4-(2-(1-(3-(5-chloro-2-(1H-tetrazol-1-yl)phenyl)acryloyl)-3-phenylpiperidin-2-yl)-1H-imidazol-4-yl)phenyl)carbamate,
1-39: (4-(2-((4-carboxyphenyl)carbamoyl)-3-phenylpiperidine-1-carbonyl)cyclohexyl)methanaminium 2,2,2-trifluoroacetate,
1-40: (E)-methyl 4-(2-(1-(3-(5-chloro-2-(1H-tetrazol-1-yl)phenyl)acryloyl)-3-phenylpiperidin-2-yl)-1H-imidazol-4-yl)benzoate,
1-41: (E)-3-(5-chloro-2-(1H-tetrazol-1-yl)phenyl)-1-(3-phenyl-2-(4-(pyridin-2-yl)-1H-imidazol-2-yl)piperidin-1-yl)prop-2-en-1-one,
1-42: (E)-3-(5-chloro-2-(1H-tetrazol-1-yl)phenyl)-1-(3-phenyl-2-(4-(pyridin-4-yl)-1H-imidazol-2-yl)piperidin-1-yl)prop-2-en-1-one,
1-43: (4-(2-((4-carboxyphenyl)carbamoyl)-3-phenylpiperidine-1-carbonyl)-2-cyclopropylphenyl)methanaminium 2,2,2-trifluoroacetate,
1-44: (E)-methyl (3-bromo-4-(2-(1-(3-(5-chloro-2-(1H-tetrazol-1-yl)phenyl)acryloyl)-3-phenylpiperidin-2-yl)-1H-imidazol-4-yl)phenyl)carbamate,
1-45: (E)-4-(2-(1-(3-(5-chloro-2-(1H-tetrazol-1-yl)phenyl)acryloyl)-3-phenylpiperidin-2-yl)-1H-imidazol-4-yl)benzoic acid,
1-46: (E)-methyl (4-(2-(1-(3-(5-chloro-2-(1H-tetrazol-1-yl)phenyl)acryloyl)-3-phenylpiperidin-2-yl)-1H-imidazol-4-yl)phenyl)carbamate,
1-47: (E)-methyl (4-(2-(1-(3-(5-chloro-2-(1H-tetrazol-1-yl)phenyl)acryloyl)-3-phenylpiperidin-2-yl)-1H-imidazol-4-yl)phenyl)carbamate.
1-48: (E)-methyl (3-amino-4-(2-(1-(3-(5-chloro-2-(1H-tetrazol-1-yl)phenyl)acryloyl)-3-phenylpiperidin-2-yl)-1H-imidazol-4-yl)phenyl)carbamate,
1-49: 4-(1-(4-(aminomethyl)cyclohexanecarbonyl)-3-phenylpiperidine-2-carboxamido)benzoic acid.
1-50: 4-(1-(4-(aminomethyl)cyclohexanecarbonyl)-3-phenyl piperidine-2-carboxamido)benzoic acid,
1-51: (E)-methyl (3-butyramido-4-(2-(1-(3-(5-chloro-2-(1H-tetrazol-1-yl)phenyl)acryloyl)-3-phenylpiperidin-2-yl)-1H-imidazol-4-yl)phenyl)carbamate,
1-52: methyl (4-(2-((2S,3S)-1-((E)-3-(5-chloro-2-(1H-tetrazol-1-yl)phenyl)acryloyl)-3-phenylpiperidin-2-yl)-5-methyl-1H-imidazol-4-yl)phenyl)carbamate,
1-53: (E)-6-(2-(1-(3-(5-chloro-2-(1H-tetrazol-1-yl)phenyl)acryloyl)-3-phenylpiperidin-2-yl)-1H-imidazol-4-yl)benzo[d]oxazol-2(3H)-one,
1-54: (E)-methyl 3-(2-(1-(3-(5-chloro-2-(1H-tetrazol-1-yl)phenyl)acryloyl)-3-phenylpiperidin-2-yl)-1H-imidazol-4-yl)benzoate,
1-55: methyl (4-(5-chloro-2-((2S,3S)-1-((E)-3-(5-chloro-2-(1H-tetrazol-1-yl)phenyl)acryloyl)-3-phenylpiperidin-2-yl)-1H-imidazol-4-yl)phenyl)carbamate,
1-56: 4-((2R,3R)-1-((E)-3-(5-chloro-2-(1H-tetrazol-1-yl)phenyl)acryloyl)-3-phenylpiperidine-2-carboxamido)benzoic acid,
1-57: 4-((2S,3S)-1-((E)-3-(5-chloro-2-(1H-tetrazol-1-yl)phenyl)acryloyl)-3-phenylpiperidine-2-carboxamido)benzoic acid,
1-58: (E)-3-(2-(1-(3-(5-chloro-2-(1H-tetrazol-1-yl)phenyl)acryloyl)-3-phenylpiperidin-2-yl)-1H-imidazol-4-yl)benzoic acid,
1-59: (E)-1-(3-(5-chloro-2-(1H-tetrazol-1-yl)phenyl)acryloyl)-N-(1H-indazol-5-yl)-3-phenylpiperidine-2-carboxamide,
1-60: (E)-1-(3-(5-chloro-2-(1H-tetrazol-1-yl)phenyl)acryloyl)-N-(1H-indazol-5-yl)-3-phenylpiperidine-2-carboxamide,
1-61: (E)-1-(3-(5-chloro-2-(1H-tetrazol-1-yl)phenyl)acryloyl)-N-(1H-indazol-6-yl)-3-phenylpiperidine-2-carboxamide,
1-62: 2-((2R,3R)-1-((E)-3-(5-chloro-2-(1H-tetrazol-1-yl)phenyl)acryloyl)-3-phenylpiperidin-2-yl)-5-(2-oxoindolin-5-yl)-1H-imidazol-3-ium 2,2,2-trifluoroacetate
1-63: 5-(2-((2S,3S)-1-((E)-3-(5-chloro-2-(1H-tetrazol-1-yl)phenyl)acryloyl)-3-phenylpiperidin-2-yl)-1H-imidazol-5-yl)indolin-2-one,
1-64: methyl (4-(5-chloro-2-((2R,3R)-1-((E)-3-(5-chloro-2-(1H-tetrazol-1-yl)phenyl)acryloyl)-3-phenylpiperidin-2-yl)-1H-imidazol-4-yl)phenyl)carbamate,
1-65: (2S,3S)-1-((E)-3-(5-chloro-2-(1H-tetrazol-1-yl)phenyl)acryloyl)-N-(1H-indazol-5-yl)-3-phenylpiperidine-2-carboxamide,
1-66: methyl (4-(2-((2S,3R)-3-(((tert-butyldimethylsilyl)oxy)methyl)-1-((E)-3-(5-chloro-2-(1H-tetrazol-1-yl)phenyl)acryloyl)piperidin-2-yl)-1H-imidazol-4-yl)phenyl)carbamate,
1-67: methyl (4-(2-((2S,3R)-1-((E)-3-(5-chloro-2-(1H-tetrazol-1-yl)phenyl)acryloyl)-3-(hydroxymethyl)piperidin-2-yl)-1H-imidazol-4-yl)phenyl)carbamate,
1-68: (E)-3-(5-chloro-2-(1H-tetrazol-1-yl)phenyl)-1-((2S,3S)-2-(5-fluoro-1H-benzo[d]imidazol-2-yl)-3-phenylpiperidin-1-yl)prop-2-en-1-one,
1-69: 3-(2-((2S,3S)-1-((E)-3-(5-chloro-2-(1H-tetrazol-1-yl)phenyl)acryloyl)-3-phenylpiperidin-2-yl)-1H-imidazol-4-yl)benzoic acid,
1-70: 3-(2-((2R,3R)-1-((E)-3-(5-chloro-2-(1H-tetrazol-1-yl)phenyl)acryloyl)-3-phenylpiperidin-2-yl)-1H-imidazol-4-yl)benzoic acid,
1-71: (E)-4-(1-(3-(5-chloro-2-(1H-tetrazol-1-yl)phenyl)acryloyl)-5-phenylpiperidine-2-carboxamido)benzoic acid, or
1-72: 4-((2S,3S)-1-((E)-3-(5-chloro-2-(1H-tetrazol-1-yl)phenyl)acryloyl)-3-phenylpiperidine-2-carboxamido)bicyclo[2.2.2]octane-1-carboxylic acid.

The structures of compounds 1-1 to 1-75 named above are shown below:

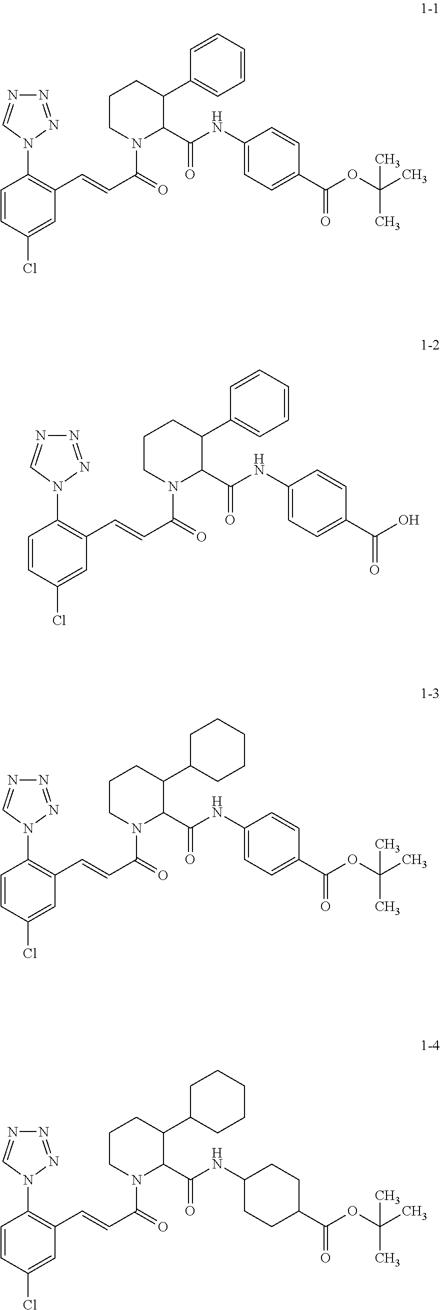 [SMILES: CC(C)(C)OC(=O)c1ccc(NC(=O)C2C(c3ccccc3)CCCN2C(=O)/C=C/c2cc(Cl)ccc2-n2cnnn2)cc1 ; O=C(O)c1ccc(NC(=O)C2C(c3ccccc3)CCCN2C(=O)/C=C/c2cc(Cl)ccc2-n2cnnn2)cc1 ; CC(C)(C)OC(=O)c1ccc(NC(=O)C2C(C3CCCCC3)CCCN2C(=O)/C=C/c2cc(Cl)ccc2-n2cnnn2)cc1 ; CC(C)(C)OC(=O)C1CCC(NC(=O)C2C(C3CCCCC3)CCCN2C(=O)/C=C/c2cc(Cl)ccc2-n2cnnn2)CC1]

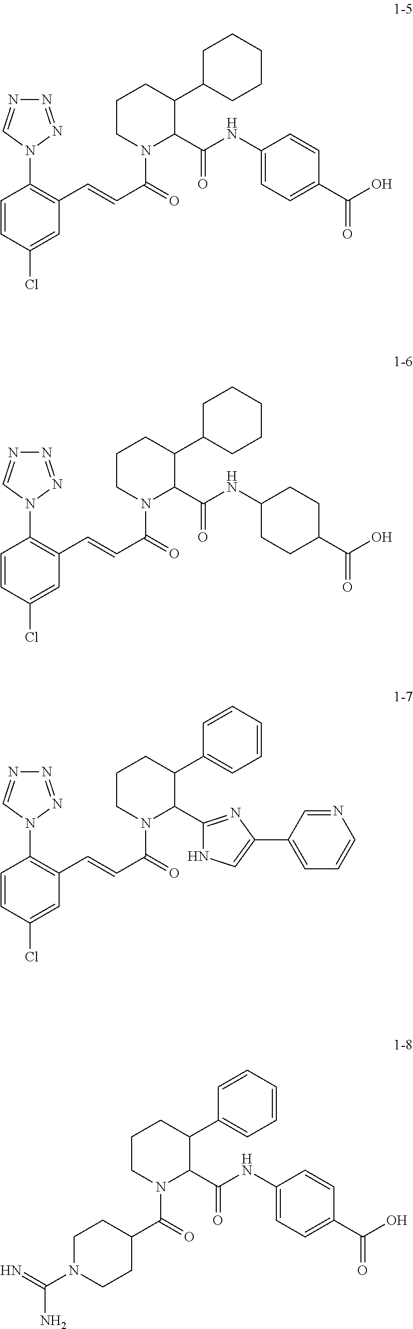 [SMILES: O=C(O)c1ccc(NC(=O)C2C(C3CCCCC3)CCCN2C(=O)/C=C/c2cc(Cl)ccc2-n2cnnn2)cc1 ; O=C(O)C1CCC(NC(=O)C2C(C3CCCCC3)CCCN2C(=O)/C=C/c2cc(Cl)ccc2-n2cnnn2)CC1 ; O=C(/C=C/c1cc(Cl)ccc1-n1cnnn1)N1CCCC(c2ccccc2)C1c1nc(-c2cccnc2)c[nH]1 ; N=C(N)N1CCC(C(=O)N2CCCC(c3ccccc3)C2C(=O)Nc2ccc(C(=O)O)cc2)CC1]

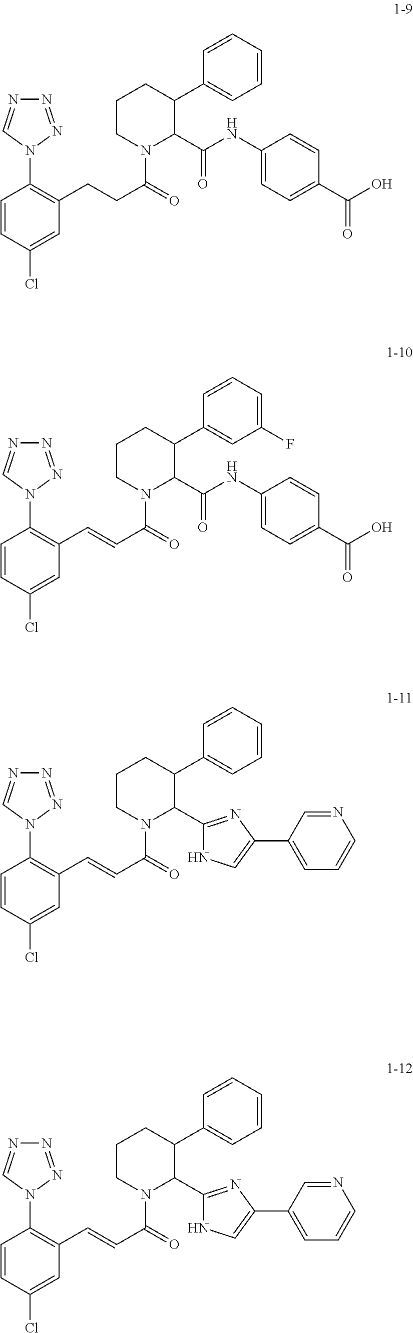 [SMILES: O=C(O)c1ccc(NC(=O)C2C(c3ccccc3)CCCN2C(=O)CCc2cc(Cl)ccc2-n2cnnn2)cc1 ; O=C(O)c1ccc(NC(=O)C2C(c3cccc(F)c3)CCCN2C(=O)/C=C/c2cc(Cl)ccc2-n2cnnn2)cc1 ; O=C(/C=C/c1cc(Cl)ccc1-n1cnnn1)N1CCCC(c2ccccc2)C1c1nc(-c2cccnc2)c[nH]1 ; O=C(/C=C/c1cc(Cl)ccc1-n1cnnn1)N1CCCC(c2ccccc2)C1c1nc(-c2cccnc2)c[nH]1]

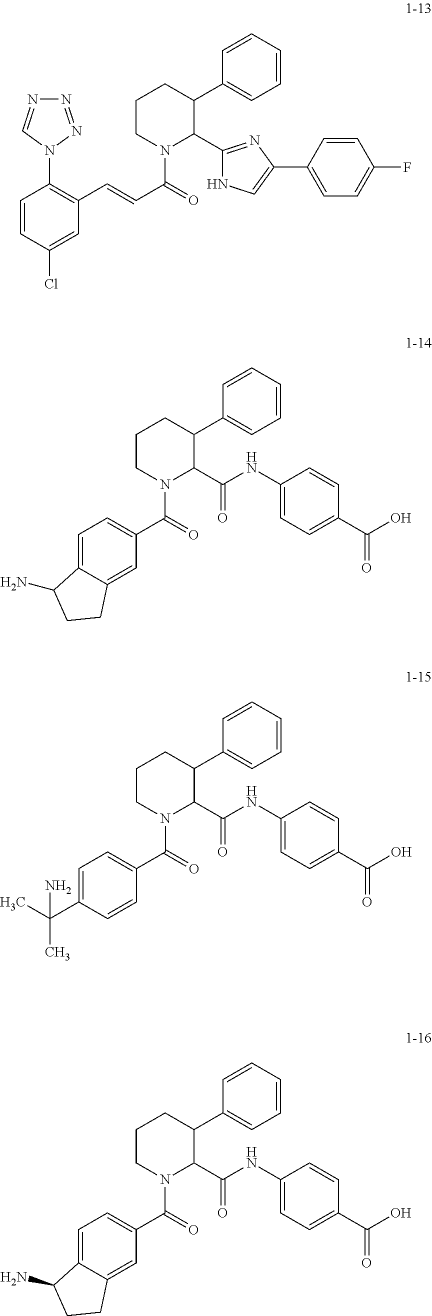 [SMILES: O=C(/C=C/c1cc(Cl)ccc1-n1cnnn1)N1CCCC(c2ccccc2)C1c1nc(-c2ccc(F)cc2)c[nH]1 ; NC1CCc2cc(C(=O)N3CCCC(c4ccccc4)C3C(=O)Nc3ccc(C(=O)O)cc3)ccc21 ; CC(C)(N)c1ccc(C(=O)N2CCCC(c3ccccc3)C2C(=O)Nc2ccc(C(=O)O)cc2)cc1 ; N[C@@H]1CCc2cc(C(=O)N3CCCC(c4ccccc4)C3C(=O)Nc3ccc(C(=O)O)cc3)ccc21]

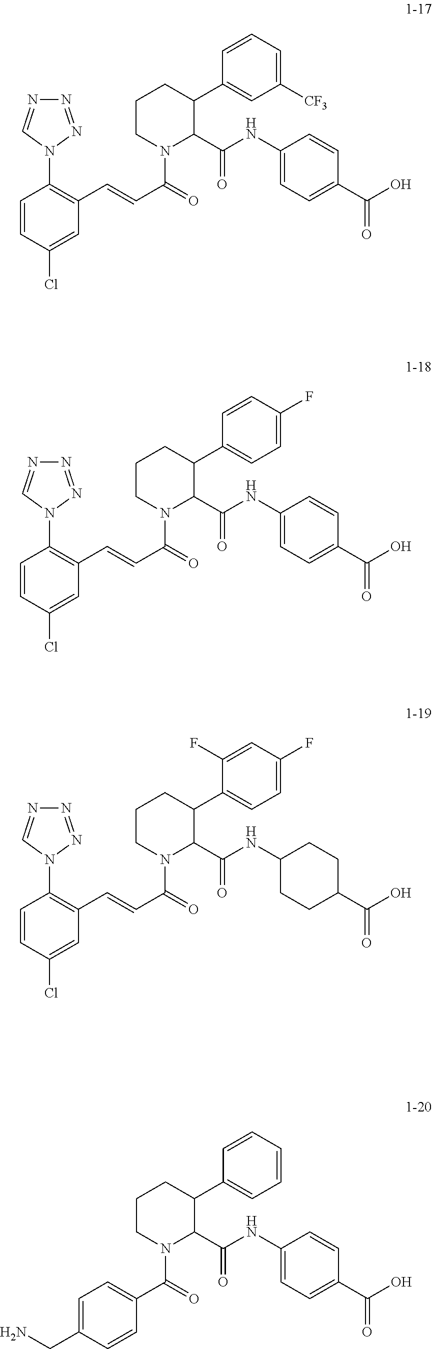 [SMILES: O=C(O)c1ccc(NC(=O)C2C(c3cccc(C(F)(F)F)c3)CCCN2C(=O)/C=C/c2cc(Cl)ccc2-n2cnnn2)cc1 ; O=C(O)c1ccc(NC(=O)C2C(c3ccc(F)cc3)CCCN2C(=O)/C=C/c2cc(Cl)ccc2-n2cnnn2)cc1 ; O=C(O)C1CCC(NC(=O)C2C(c3ccc(F)cc3F)CCCN2C(=O)/C=C/c2cc(Cl)ccc2-n2cnnn2)CC1 ; NCc1ccc(C(=O)N2CCCC(c3ccccc3)C2C(=O)Nc2ccc(C(=O)O)cc2)cc1]

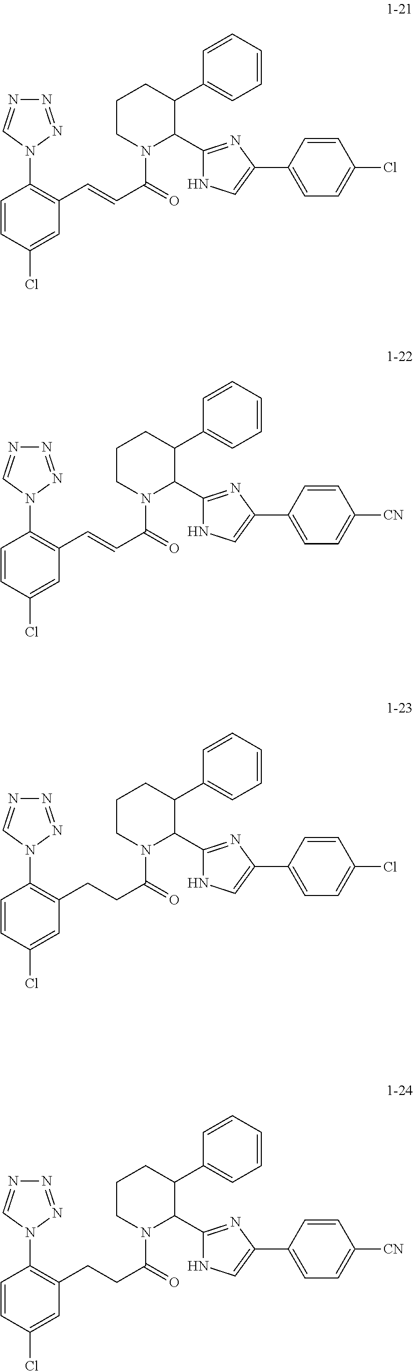 [SMILES: O=C(/C=C/c1cc(Cl)ccc1-n1cnnn1)N1CCCC(c2ccccc2)C1c1nc(-c2ccc(Cl)cc2)c[nH]1 ; N#Cc1ccc(-c2c[nH]c(C3C(c4ccccc4)CCCN3C(=O)/C=C/c3cc(Cl)ccc3-n3cnnn3)n2)cc1 ; O=C(CCc1cc(Cl)ccc1-n1cnnn1)N1CCCC(c2ccccc2)C1c1nc(-c2ccc(Cl)cc2)c[nH]1 ; N#Cc1ccc(-c2c[nH]c(C3C(c4ccccc4)CCCN3C(=O)CCc3cc(Cl)ccc3-n3cnnn3)n2)cc1]

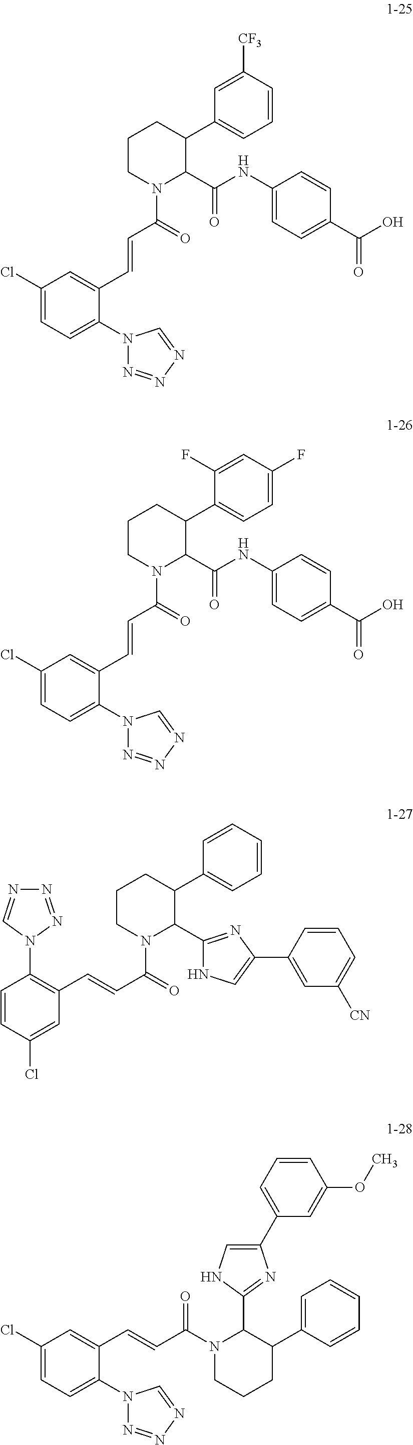 [SMILES: O=C(O)c1ccc(NC(=O)C2C(c3cccc(C(F)(F)F)c3)CCCN2C(=O)/C=C/c2cc(Cl)ccc2-n2cnnn2)cc1 ; O=C(O)c1ccc(NC(=O)C2C(c3ccc(F)cc3F)CCCN2C(=O)/C=C/c2cc(Cl)ccc2-n2cnnn2)cc1 ; N#Cc1cccc(-c2c[nH]c(C3C(c4ccccc4)CCCN3C(=O)/C=C/c3cc(Cl)ccc3-n3cnnn3)n2)c1 ; COc1cccc(-c2c[nH]c(C3C(c4ccccc4)CCCN3C(=O)/C=C/c3cc(Cl)ccc3-n3cnnn3)n2)c1]

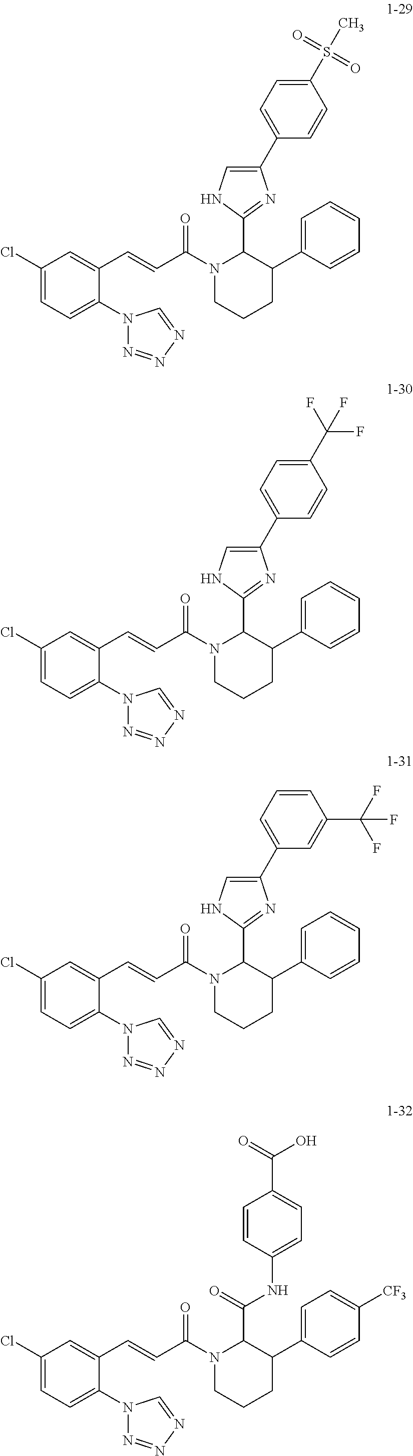 [SMILES: CS(=O)(=O)c1ccc(-c2c[nH]c(C3C(c4ccccc4)CCCN3C(=O)/C=C/c3cc(Cl)ccc3-n3cnnn3)n2)cc1 ; O=C(/C=C/c1cc(Cl)ccc1-n1cnnn1)N1CCCC(c2ccccc2)C1c1nc(-c2ccc(C(F)(F)F)cc2)c[nH]1 ; O=C(/C=C/c1cc(Cl)ccc1-n1cnnn1)N1CCCC(c2ccccc2)C1c1nc(-c2cccc(C(F)(F)F)c2)c[nH]1 ; O=C(O)c1ccc(NC(=O)C2C(c3ccc(C(F)(F)F)cc3)CCCN2C(=O)/C=C/c2cc(Cl)ccc2-n2cnnn2)cc1]

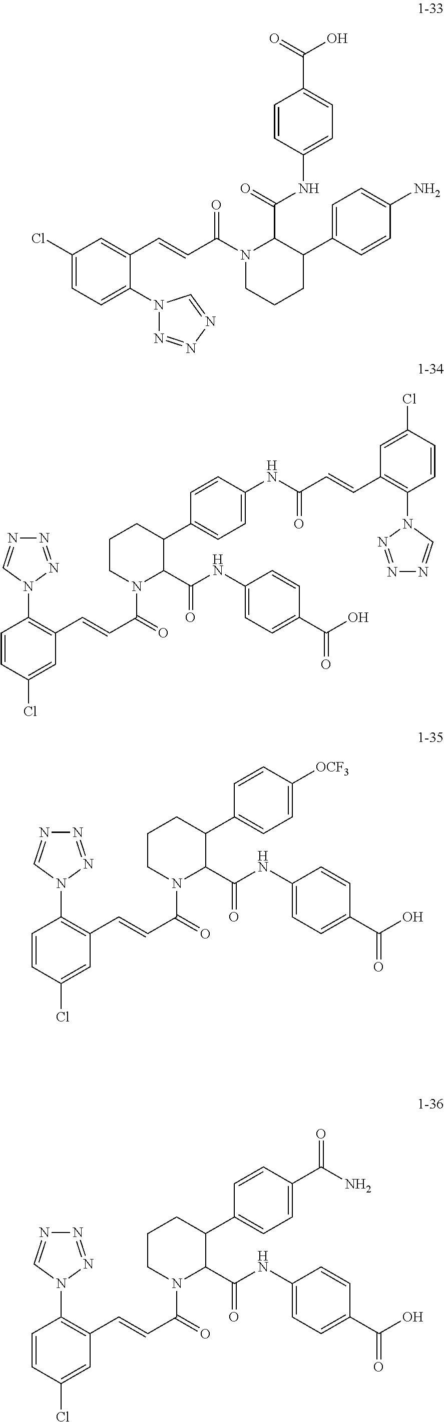 [SMILES: Nc1ccc(C2CCCN(C(=O)/C=C/c3cc(Cl)ccc3-n3cnnn3)C2C(=O)Nc2ccc(C(=O)O)cc2)cc1 ; O=C(/C=C/c1cc(Cl)ccc1-n1cnnn1)Nc1ccc(C2CCCN(C(=O)/C=C/c3cc(Cl)ccc3-n3cnnn3)C2C(=O)Nc2ccc(C(=O)O)cc2)cc1 ; O=C(O)c1ccc(NC(=O)C2C(c3ccc(OC(F)(F)F)cc3)CCCN2C(=O)/C=C/c2cc(Cl)ccc2-n2cnnn2)cc1 ; NC(=O)c1ccc(C2CCCN(C(=O)/C=C/c3cc(Cl)ccc3-n3cnnn3)C2C(=O)Nc2ccc(C(=O)O)cc2)cc1]

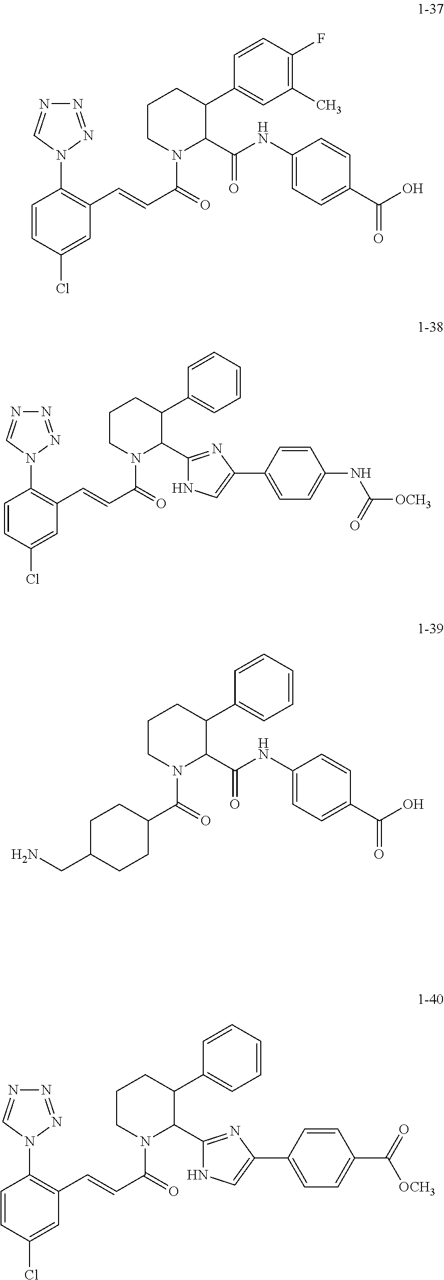 [SMILES: Cc1cc(C2CCCN(C(=O)/C=C/c3cc(Cl)ccc3-n3cnnn3)C2C(=O)Nc2ccc(C(=O)O)cc2)ccc1F ; COC(=O)Nc1ccc(-c2c[nH]c(C3C(c4ccccc4)CCCN3C(=O)/C=C/c3cc(Cl)ccc3-n3cnnn3)n2)cc1 ; NCC1CCC(C(=O)N2CCCC(c3ccccc3)C2C(=O)Nc2ccc(C(=O)O)cc2)CC1 ; COC(=O)c1ccc(-c2c[nH]c(C3C(c4ccccc4)CCCN3C(=O)/C=C/c3cc(Cl)ccc3-n3cnnn3)n2)cc1]

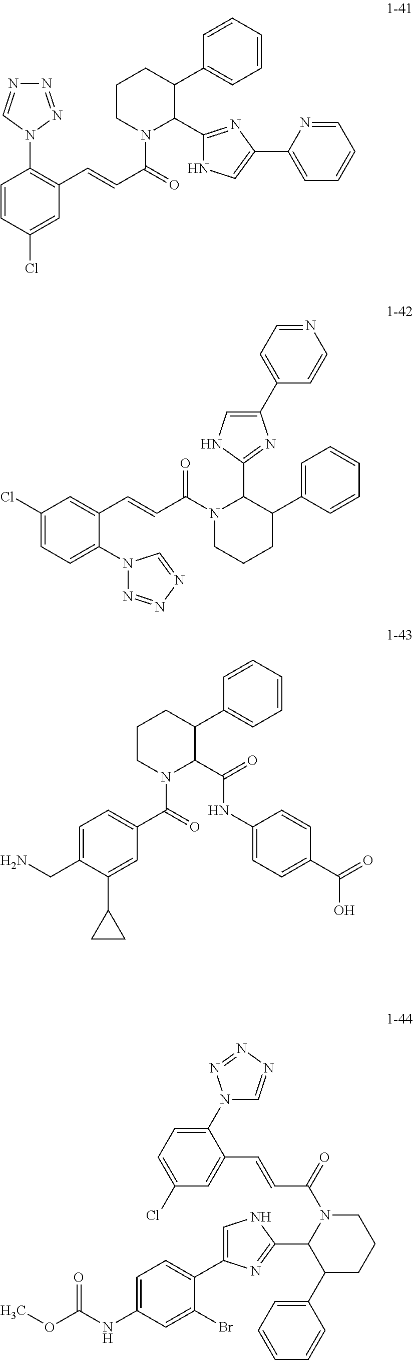 [SMILES: O=C(/C=C/c1cc(Cl)ccc1-n1cnnn1)N1CCCC(c2ccccc2)C1c1nc(-c2ccccn2)c[nH]1 ; O=C(/C=C/c1cc(Cl)ccc1-n1cnnn1)N1CCCC(c2ccccc2)C1c1nc(-c2ccncc2)c[nH]1 ; NCc1ccc(C(=O)N2CCCC(c3ccccc3)C2C(=O)Nc2ccc(C(=O)O)cc2)cc1C1CC1 ; COC(=O)Nc1ccc(-c2c[nH]c(C3C(c4ccccc4)CCCN3C(=O)/C=C/c3cc(Cl)ccc3-n3cnnn3)n2)c(Br)c1]

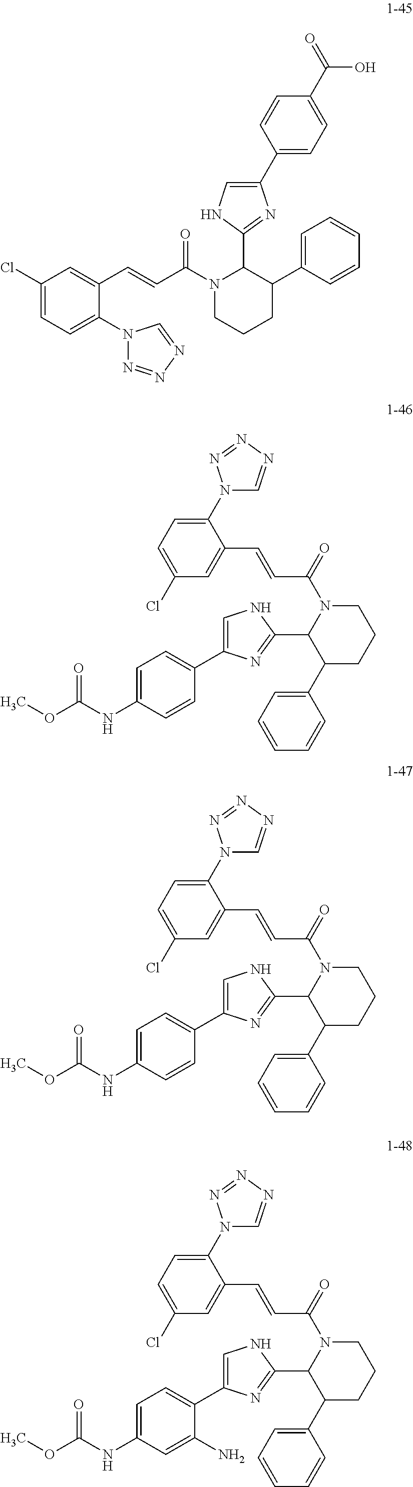 [SMILES: O=C(O)c1ccc(-c2c[nH]c(C3C(c4ccccc4)CCCN3C(=O)/C=C/c3cc(Cl)ccc3-n3cnnn3)n2)cc1 ; COC(=O)Nc1ccc(-c2c[nH]c(C3C(c4ccccc4)CCCN3C(=O)/C=C/c3cc(Cl)ccc3-n3cnnn3)n2)cc1 ; COC(=O)Nc1ccc(-c2c[nH]c(C3C(c4ccccc4)CCCN3C(=O)/C=C/c3cc(Cl)ccc3-n3cnnn3)n2)cc1 ; COC(=O)Nc1ccc(-c2c[nH]c(C3C(c4ccccc4)CCCN3C(=O)/C=C/c3cc(Cl)ccc3-n3cnnn3)n2)c(N)c1]

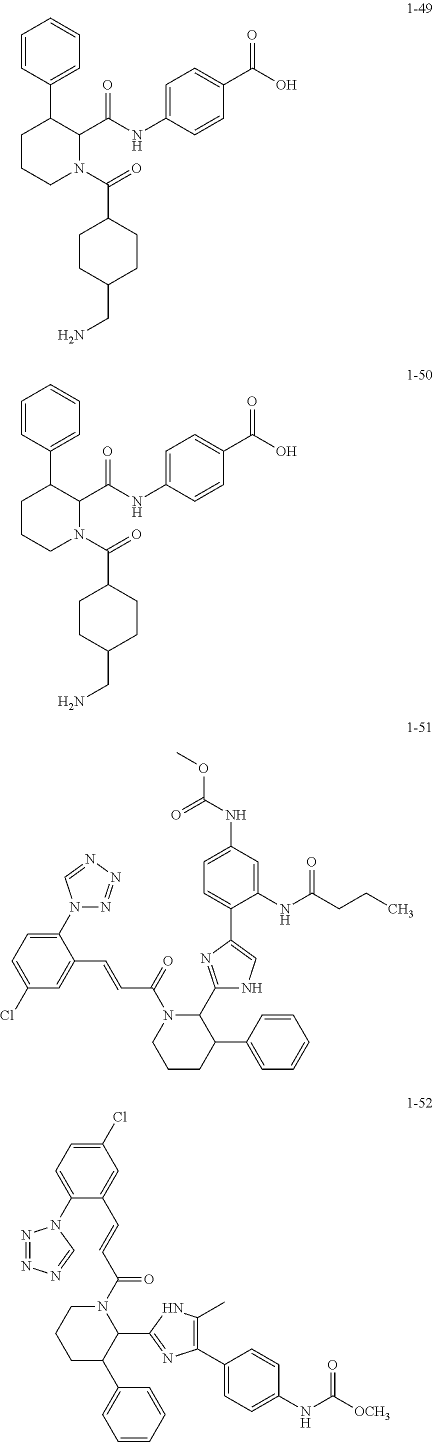 [SMILES: NCC1CCC(C(=O)N2CCCC(c3ccccc3)C2C(=O)Nc2ccc(C(=O)O)cc2)CC1 ; NCC1CCC(C(=O)N2CCCC(c3ccccc3)C2C(=O)Nc2ccc(C(=O)O)cc2)CC1 ; CCCC(=O)Nc1cc(NC(=O)OC)ccc1-c1c[nH]c(C2C(c3ccccc3)CCCN2C(=O)/C=C/c2cc(Cl)ccc2-n2cnnn2)n1 ; COC(=O)Nc1ccc(-c2nc(C3C(c4ccccc4)CCCN3C(=O)/C=C/c3cc(Cl)ccc3-n3cnnn3)[nH]c2C)cc1]

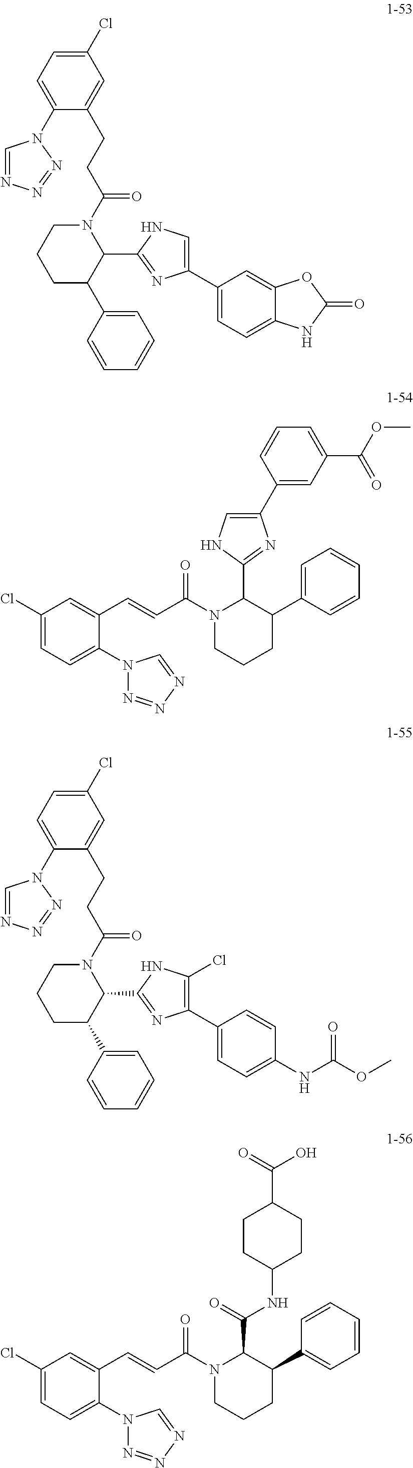 [SMILES: O=C(CCc1cc(Cl)ccc1-n1cnnn1)N1CCCC(c2ccccc2)C1c1nc(-c2ccc3[nH]c(=O)oc3c2)c[nH]1 ; COC(=O)c1cccc(-c2c[nH]c(C3C(c4ccccc4)CCCN3C(=O)/C=C/c3cc(Cl)ccc3-n3cnnn3)n2)c1 ; COC(=O)Nc1ccc(-c2nc([C@@H]3[C@H](c4ccccc4)CCCN3C(=O)CCc3cc(Cl)ccc3-n3cnnn3)[nH]c2Cl)cc1 ; O=C(O)C1CCC(NC(=O)[C@H]2[C@@H](c3ccccc3)CCCN2C(=O)/C=C/c2cc(Cl)ccc2-n2cnnn2)CC1]

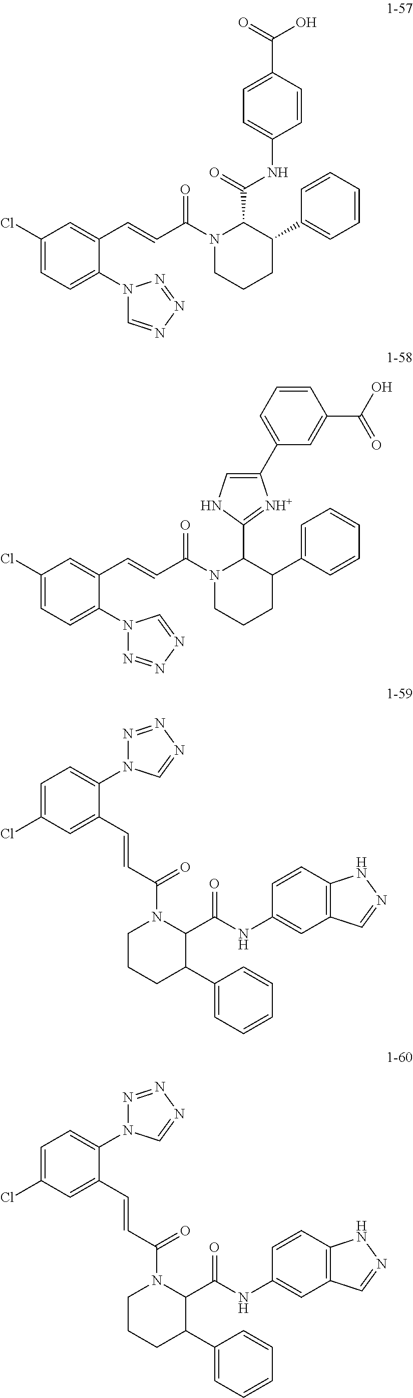 [SMILES: O=C(O)c1ccc(NC(=O)[C@@H]2[C@H](c3ccccc3)CCCN2C(=O)/C=C/c2cc(Cl)ccc2-n2cnnn2)cc1 ; O=C(O)c1cccc(-c2c[nH]c(C3C(c4ccccc4)CCCN3C(=O)/C=C/c3cc(Cl)ccc3-n3cnnn3)[nH+]2)c1 ; O=C(Nc1ccc2[nH]ncc2c1)C1C(c2ccccc2)CCCN1C(=O)/C=C/c1cc(Cl)ccc1-n1cnnn1 ; O=C(Nc1ccc2[nH]ncc2c1)C1C(c2ccccc2)CCCN1C(=O)/C=C/c1cc(Cl)ccc1-n1cnnn1]

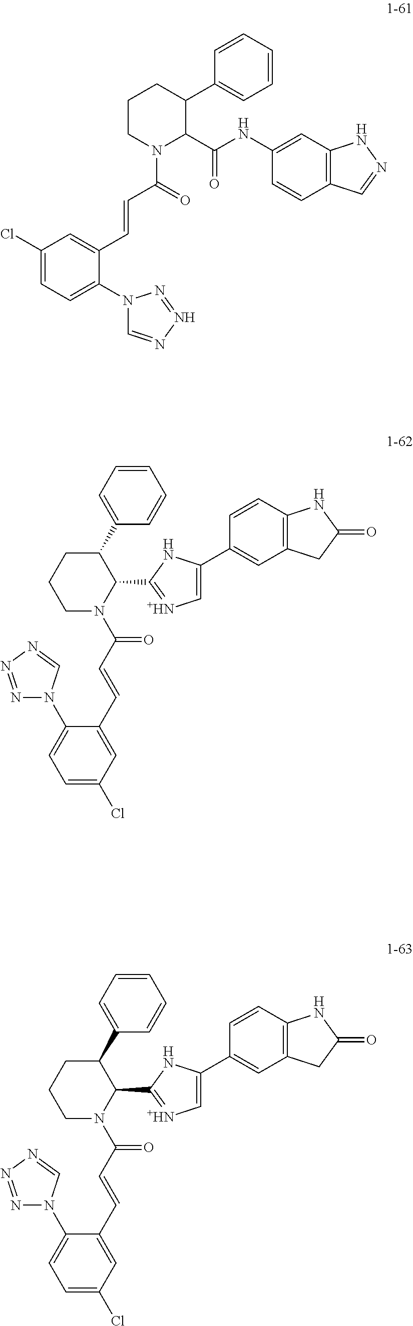 [SMILES: O=C(Nc1ccc2cn[nH]c2c1)C1C(c2ccccc2)CCCN1C(=O)/C=C/c1cc(Cl)ccc1-n1cnnn1 ; O=C1Cc2cc(-c3c[nH+]c([C@H]4[C@@H](c5ccccc5)CCCN4C(=O)/C=C/c4cc(Cl)ccc4-n4cnnn4)[nH]3)ccc2N1 ; O=C1Cc2cc(-c3c[nH+]c([C@@H]4[C@H](c5ccccc5)CCCN4C(=O)/C=C/c4cc(Cl)ccc4-n4cnnn4)[nH]3)ccc2N1]

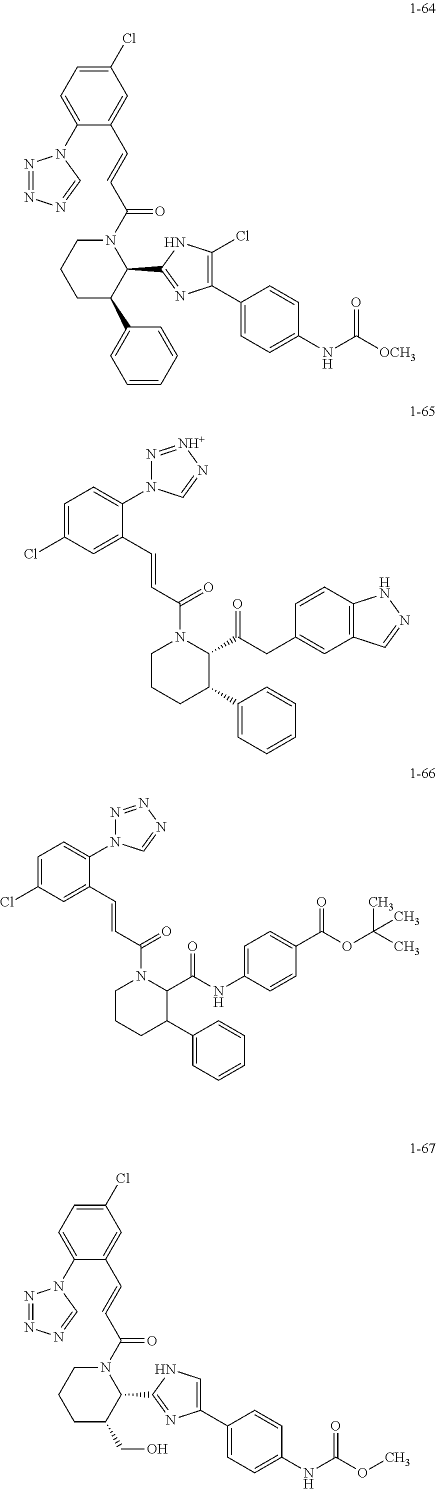 [SMILES: COC(=O)Nc1ccc(-c2nc([C@H]3[C@@H](c4ccccc4)CCCN3C(=O)/C=C/c3cc(Cl)ccc3-n3cnnn3)[nH]c2Cl)cc1 ; O=C(Cc1ccc2[nH]ncc2c1)[C@@H]1[C@H](c2ccccc2)CCCN1C(=O)/C=C/c1cc(Cl)ccc1-n1cn[nH+]n1 ; CC(C)(C)OC(=O)c1ccc(NC(=O)C2C(c3ccccc3)CCCN2C(=O)/C=C/c2cc(Cl)ccc2-n2cnnn2)cc1 ; COC(=O)Nc1ccc(-c2c[nH]c([C@@H]3[C@H](CO)CCCN3C(=O)/C=C/c3cc(Cl)ccc3-n3cnnn3)n2)cc1]

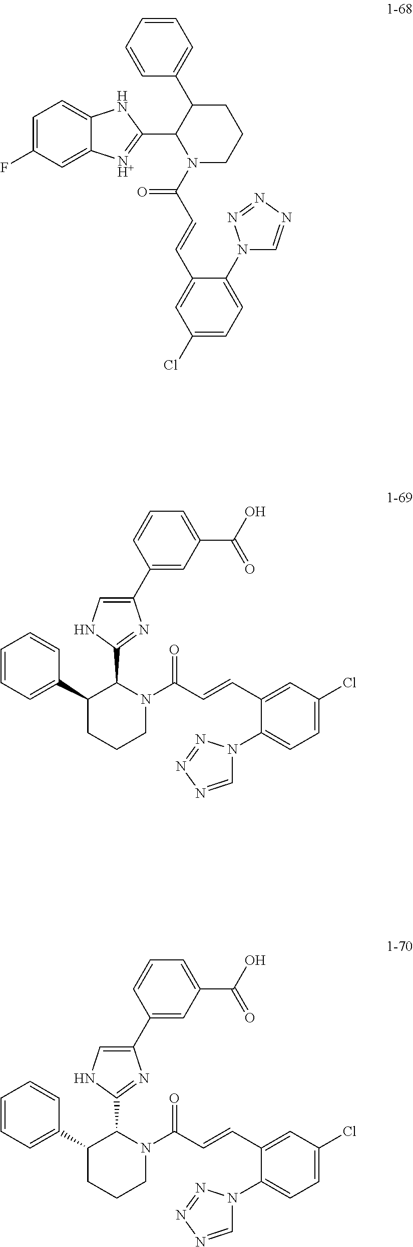 [SMILES: O=C(/C=C/c1cc(Cl)ccc1-n1cnnn1)N1CCCC(c2ccccc2)C1c1[nH]c2ccc(F)cc2[nH+]1 ; O=C(O)c1cccc(-c2c[nH]c([C@@H]3[C@H](c4ccccc4)CCCN3C(=O)/C=C/c3cc(Cl)ccc3-n3cnnn3)n2)c1 ; O=C(O)c1cccc(-c2c[nH]c([C@H]3[C@@H](c4ccccc4)CCCN3C(=O)/C=C/c3cc(Cl)ccc3-n3cnnn3)n2)c1]

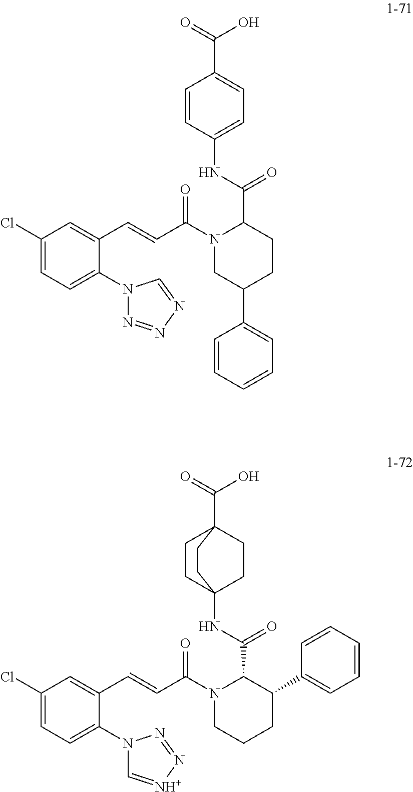 [SMILES: O=C(O)c1ccc(NC(=O)C2CCC(c3ccccc3)CN2C(=O)/C=C/c2cc(Cl)ccc2-n2cnnn2)cc1 ; O=C(NC12CCC(C(=O)O)(CC1)CC2)[C@@H]1[C@H](c2ccccc2)CCCN1C(=O)/C=C/c1cc(Cl)ccc1-n1c[nH+]nn1]

Reference to the preferred classes and subclasses set forth above is meant to include all combinations of particular and preferred groups unless stated otherwise.

Specific embodiments of the present invention include, but are not limited to the compounds identified herein as Examples 1 to 76, or pharmaceutically acceptable salts thereof

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application. These and other aspects of the invention will be apparent from the teachings contained herein.

The invention also includes compositions for inhibiting loss of blood platelets, inhibiting formation of blood platelet aggregates, inhibiting formation of fibrin, inhibiting thrombus formation, inhibiting embolus formation, and treating inflammatory disorders in a mammal, comprising a compound of the invention in a pharmaceutically acceptable carrier. These compositions may optionally include anticoagulants, antiplatelet agents, and thrombolytic agents. The compositions can be added to blood, blood products, or mammalian organs in order to effect the desired inhibitions.

The invention also includes compositions for preventing or treating unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels, in a mammal, comprising a compound of the invention in a pharmaceutically acceptable carrier. These compositions may optionally include anticoagulants, antiplatelet agents, and thrombolytic agents.

The invention also includes a method for reducing the thrombogenicity of a surface in a mammal by attaching to the surface, either covalently or noncovalently, a compound of the invention.

Compounds of the invention are Factor XIa inhibitors and may have therapeutic value in, for example, preventing coronary artery disease. The compounds are selective Factor XIa inhibitors or dual inhibitors of Factor XIa and plasma kallikrein.

It will be understood that, as used herein, references to the compounds of structural Formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Also included are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

The present invention encompasses all stereoisomeric forms of the compounds of Formula I. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both enantiomers and mixtures thereof, are embraced within the Formula. When a particular configuration is depicted, that entantiomer (either (R) or (S), at that center) is intended. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

When any variable (e.g. R⁴, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Reference to the compounds of this invention as those of a specific formula or embodiment, e.g., Formula I or any other generic structural formula or specific compound described or claimed herein, is intended to encompass the specific compound or compounds falling within the scope of the formula or embodiment, including salts thereof, particularly pharmaceutically acceptable salts, solvates of such compounds and solvated salt forms thereof, where such forms are possible unless specified otherwise.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁₋₆alkyl esters and —C₁₋₆alkyl substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

Except where noted herein, the term “alkyl” is intended to include both branched- and straight-chain saturated aliphatic hydrocarbon groups having the specified number of carbon atoms. Commonly used abbreviations for alkyl groups are used throughout the specification, e.g. methyl, may be represented by conventional abbreviations including “Me” or CH₃ or a symbol that is an extended bond as the terminal group, e.g.

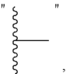

ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₄ alkyl” (or “C₁-C₄ alkyl”) for example, means linear or branched chain alkyl groups, including all isomers, having the specified number of carbon atoms. For example, the structure

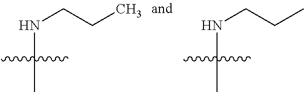

have equivalent meanings. C₁₋₄ alkyl includes n-, iso-, sec- and t-butyl, n- and isopropyl, ethyl and methyl. If no number is specified, 1-4 carbon atoms are intended for linear or branched alkyl groups.

Except where noted herein, “alkanol” is intended to include aliphatic alcohols having the specified number of carbon atoms, such as methanol, ethanol, propanol, etc., where the —OH group is attached at any aliphatic carbon, e.g., propan-1-ol, propan-2-ol, etc.

Except where noted, the term “cycloalkyl” means a monocyclic or bicyclic saturated aliphatic hydrocarbon group having the specified number of carbon atoms. For example, “cycloalkyl” includes cyclopropyl, cyclobutyl, cyclopentyl, cyclohexyl, and so on.

Except where noted, the term “halogen” or “halo” means fluorine, chlorine, bromine or iodine.

Except where noted, the term “heteroaryl”, as used herein, represents a stable monocyclic, bicyclic or tricyclic ring of up to 10 atoms in each ring, wherein at least one ring is aromatic, and at least one ring contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, tetra-hydroquinoline and 3-oxo-3,4-dihydro-2Nbenzo[b][1,4]thiazine. If the heteroaryl contains nitrogen atoms, it is understood that the corresponding N-oxides thereof are also encompassed by this definition.

Except where noted, the term “heterocycle” or “heterocyclyl” as used herein is intended to mean a 5- to 10-membered nonaromatic ring, unless otherwise specified, containing from 1 to 4 heteroatoms selected from the group consisting of O, N, S, SO, or SO₂ and includes bicyclic groups. “Heterocyclyl” therefore includes, but is not limited to the following: piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

Except where noted, the term “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl and indanyl.

The term “saturated heterocycle” refers to a saturated monocyclic 5- to 8-membered ring having 1-4 heteroatoms selected from N, O and S, or a 7- to 12-membered saturated bicyclic ring system having 1-6 heteroatoms selected from N, O and S, or a 12- to 14-membered ring having 1-4 heteroatoms selected from N, O and S. Representative examples include piperidinyl, piperazinyl, azepanyl, pyrrolidinyl, pyrazolidinyl, imidazolidinyl, oxazolidinyl, isoxazolidinyl, morpholinyl, thiomorpholinyl, thiazolidinyl, isothiazolidinyl, and tetrahydrofuryl (or tetrahydrofuranyl).

Except where noted herein, the term “carbocycle” (and variations thereof such as “carbocyclic” or “carbocyclyl”) as used herein, unless otherwise indicated, refers to a C₃ to C₈ monocyclic saturated or unsaturated ring, e.g., C₃₋₈ monocyclic carbocycle, or a C₉ to C₁₂ bicyclic saturated or unsaturated ring, e.g., C₉₋₁₂ bicyclic carbocycle. The carbocycle may be attached to the rest of the molecule at any carbon atom which results in a stable compound.

Saturated carbocyclic rings include, for example, “cycloalkyl” rings, e.g., cyclopropyl, cyclobutyl, etc. Unsaturated carbocyclic rings include, for example, “aryl” rings. Unsaturated bicyclic carbocyclic ring systems include fused ring systems where all ring system members are carbon atoms and where at least one of the fused rings is not saturated.

“Celite®” (Fluka) diatomite is diatomaceous earth, and can be referred to as “celite”.

Except where noted herein, structures containing substituent variables such as variable “R” below:

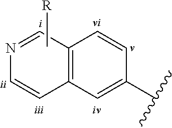

which are depicted as not being attached to any one particular bicyclic ring carbon atom, represent structures in which the variable can be optionally attached to any bicyclic ring carbon atom. For example, variable R shown in the above structure can be attached to any one of 6 bicyclic ring carbon atoms i, ii, iii, iv, v or vi.

Except where noted herein, bicyclic ring systems include fused ring systems, where two rings share two atoms, and spiro ring systems, where two rings share one atom.

The invention also includes derivatives of the compounds of Formula I, acting as prodrugs and solvates. Prodrugs, following administration to the patient, are converted in the body by normal metabolic or chemical processes, such as through hydrolysis in the blood, to the compound of Formula 1. Such prodrugs include those that demonstrate enhanced bioavailability, tissue specificity, and/or cellular delivery, to improve drug absorption of the compound of Formula I. The effect of such prodrugs may result from modification of physicochemical properties such as lipophilicity, molecular weight, charge, and other physicochemical properties that determine the permeation properties of the drug.

The preparation of pharmaceutically acceptable salts from compounds of the Formula I capable of salt formation, including their stereoisomeric forms is carried out in a manner known per se. With basic reagents such as hydroxides, carbonates, hydrogencarbonates, alkoxides and ammonia or organic bases, for example, trimethyl- or triethylamine, ethanolamine, diethanolamine or triethanolamine, trometamol or alternatively basic amino acids, for example lysine, ornithine or arginine, the compounds of the Formula I form stable alkali metal, alkaline earth metal or optionally substituted ammonium salts. If the compounds of the Formula I have basic groups, stable acid addition salts can also be prepared using strong acids. For this, inorganic and organic acids such as hydrochloric, hydrobromic, sulfuric, hemisulfuric, phosphoric, methanesulfonic, benzenesulfonic, p-toluenesulfonic, 4-bromobenzenesulfonic, cyclohexylamidosulfonic, trifluoromethylsulfonic, 2-hydroxyethanesulfonic, acetic, oxalic, tartaric, succinic, glycerolphosphoric, lactic, malic, adipic, citric, fumaric, maleic, gluconic, glucuronic, palmitic or trifluoroacetic acid are suitable.

The invention also relates to medicaments containing at least one compound of the Formula I and/or of a pharmaceutically acceptable salt of the compound of the Formula I and/or an optionally stereoisomeric form of the compound of the Formula I or a pharmaceutically acceptable salt of the stereoisomeric form of the compound of Formula I, together with a pharmaceutically suitable and pharmaceutically acceptable vehicle, additive and/or other active substances and auxiliaries.

Anticoagulant therapy is indicated for the treatment and prevention of a variety of thrombotic conditions, particularly coronary artery and cerebrovascular disease. Those experienced in this field are readily aware of the circumstances requiring anticoagulant therapy. The term “patient” used herein is taken to mean mammals such as primates, humans, sheep, horses, cattle, pigs, dogs, cats, rats, and mice.

Factor XIa or dual Factor XIa/plasma kallikrein inhibition are useful not only in the anticoagulant therapy of individuals having thrombotic conditions, but are useful whenever inhibition of blood coagulation is required such as to prevent coagulation of stored whole blood and to prevent coagulation in other biological samples for testing or storage. Thus, the Factor XIa or dual Factor XIa/plasma kallikrein inhibitors can be added to or contacted with any medium containing or suspected of containing thrombin and in which it is desired that blood coagulation be inhibited, e.g., when contacting the mammal's blood with material selected from the group consisting of vascular grafts, stents, orthopedic prosthesis, cardiac prosthesis, and extracorporeal circulation systems.

Compounds of the invention may be useful for treating or preventing venous thromboembolism (e.g., obstruction or occlusion of a vein by a detached thrombus; obstruction or occlusion of a lung artery by a detached thrombus), cardiogenic thromboembolism (e.g., obstruction or occlusion of the heart by a detached thrombus), arterial thrombosis (e.g., formation of a thrombus within an artery that may cause infarction of tissue supplied by the artery), atherosclerosis (e.g., arteriosclerosis characterized by irregularly distributed lipid deposits) in mammals, and for lowering the propensity of devices that come into contact with blood to clot blood.

Examples of venous thromboembolism which may be treated or prevented with compounds of the invention include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the invention may be useful for maintaining patency of indwelling catheters.

Examples of cardiogenic thromboembolism which may be treated or prevented with compounds of the invention include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of arterial thrombosis include unstable angina (severe constrictive pain in chest of coronary origin), myocardial infarction (heart muscle cell death resulting from insufficient blood supply), ischemic heart disease (local anemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterial thrombosis, compounds of the invention may be useful for maintaining patency in arteriovenous cannulas.

Examples of atherosclerosis include arteriosclerosis.

The compounds of the invention may also be kallikrein inhibitors and especially useful for treatment of hereditary angioedema.

Examples of devices that come into contact with blood include vascular grafts, stents, orthopedic prosthesis, cardiac prosthesis, and extracorporeal circulation systems.

The medicaments according to the invention can be administered by oral, inhalative, rectal or transdermal administration or by subcutaneous, intraarticular, intraperitoneal or intravenous injection. Oral administration is preferred. Coating of stents with compounds of the Formula (I) and other surfaces which come into contact with blood in the body is possible.

The invention also relates to a process for the production of a medicament, which comprises bringing at least one compound of the Formula (I) into a suitable administration form using a pharmaceutically suitable and pharmaceutically acceptable carrier and optionally further suitable active substances, additives or auxiliaries.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the Factor XIa inhibitors or dual Factor XIa/plasma kallikrein inhibitors is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the Factor XIa inhibitors or dual Factor XIa/plasma kallikrein inhibitors, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the Factor XIa inhibitors may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver between 0.025-7.5 mg/kg/day, preferably 0.1-2.5 mg/kg/day, and more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/mL, e.g. 0.1 mg/mL, 0.3 mg/mL, and 0.6 mg/mL, and administered in amounts per day of between 0.01 mL/kg patient weight and 10.0 mL/kg patient weight, e.g. 0.1 mL/kg, 0.2 mL/kg, 0.5 mL/kg. In one example, an 80 kg patient, receiving 8 mL twice a day of an intravenous formulation having a concentration of active ingredient of 0.5 mg/mL, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

Compounds of the Formula I can be administered both as a monotherapy and in combination with other therapeutic agents, including antithrombotics (anticoagulants and platelet aggregation inhibitors), thrombolytics (plasminogen activators), other profibrinolytically active substances, hypotensives, blood sugar regulators, lipid-lowering agents and antiarrhythmics.

The Factor XIa inhibitors or dual Factor XIa/plasma kallikrein inhibitors can also be co-administered with suitable anticoagulants, including, but not limited to, other Factor XIa inhibitors, thrombin inhibitors, thrombin receptor antagonists, factor VIIa inhibitors, factor Xa inhibitors, factor IXa inhibitors, factor XIIa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis), other anticoagulants such as aspirin, and thrombolytic agents such as plasminogen activators or streptokinase to achieve synergistic effects in the treatment of various vascular pathologies. Such anticoagulants include, for example, apixaban, dabigatran, cangrelor, ticagrelor, vorapaxar, clopidogrel, edoxaban, mipomersen, prasugrel, rivaroxaban, and semuloparin. For example, patients suffering from coronary artery disease, and patients subjected to angioplasty procedures, would benefit from coadministration of fibrinogen receptor antagonists and thrombin inhibitors. Factor XIa inhibitors may be administered first following thrombus formation, and tissue plasminogen activator or other plasminogen activator is administered thereafter.

Alternatively or additionally, one or more additional pharmacologically active agents may be administered in combination with a compound of the invention. The additional active agent (or agents) is intended to mean a pharmaceutically active agent (or agents) that is active in the body, including pro-drugs that convert to pharmaceutically active form after administration, which is different from the compound of the invention, and also includes free-acid, free-base and pharmaceutically acceptable salts of said additional active agents when such forms are sold commercially or are otherwise chemically possible. Generally, any suitable additional active agent or agents, including but not limited to anti-hypertensive agents, additional diuretics, anti-atherosclerotic agents such as a lipid modifying compound, anti-diabetic agents and/or anti-obesity agents may be used in any combination with the compound of the invention in a single dosage formulation (a fixed dose drug combination), or may be administered to the patient in one or more separate dosage formulations which allows for concurrent or sequential administration of the active agents (co-administration of the separate active agents). Examples of additional active agents which may be employed include but are not limited to angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril); angiotensin II receptor antagonists also known as angiotensin receptor blockers or ARBs, which may be in free-base, free-acid, salt or pro-drug form, such as azilsartan, e.g., azilsartan medoxomil potassium (EDARBI®), candesartan, e.g., candesartan cilexetil (ATACAND®), eprosartan, e.g., eprosartan mesylate (TEVETAN®), irbesartan (AVAPRO®), losartan, e.g., losartan potassium (COZAAR®), olmesartan, e.g, olmesartan medoximil (BENICAR®), telmisartan (MICARDIS®), valsartan (DIOVAN®), and any of these drugs used in combination with a thiazide-like diuretic such as hydrochlorothiazide (e.g., HYZAAR®, DIOVAN HCT®, ATACAND HCT®), etc.); potassium sparing diuretics such as amiloride HCl, spironolactone, epleranone, triamterene, each with or without HCTZ; neutral endopeptidase inhibitors (e.g., thiorphan and phosphoramidon); aldosterone antagonists; aldosterone synthase inhibitors; renin inhibitors; enalkrein; RO 42-5892; A 65317; CP 80794; ES 1005; ES 8891; SQ 34017; aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635); endothelin receptor antagonists; vasodilators (e.g. nitroprusside); calcium channel blockers (e.g., amlodipine, nifedipine, verapamil, diltiazem, felodipine, gallopamil, niludipine, nimodipine, nicardipine); potassium channel activators (e.g., nicorandil, pinacidil, cromakalim, minoxidil, aprilkalim, loprazolam); sympatholitics; beta-adrenergic blocking drugs (e.g., acebutolol, atenolol, betaxolol, bisoprolol, carvedilol, metoprolol, metoprolol tartate, nadolol, propranolol, sotalol, timolol); alpha adrenergic blocking drugs (e.g., doxazosin, prazosin or alpha methyldopa); central alpha adrenergic agonists; peripheral vasodilators (e.g. hydralazine); lipid lowering agents, e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), and fluvastatin (particularly the sodium salt sold in LESCOL®); a cholesterol absorption inhibitor such as ezetimibe (ZETIA®), and ezetimibe in combination with any other lipid lowering agents such as the HMG-CoA reductase inhibitors noted above and particularly with simvastatin (VYTORIN®) or with atorvastatin calcium; niacin in immediate-release or controlled release forms, and particularly niacin in combination with a DP antagonist such as laropiprant and/or with an HMVG-CoA reductase inhibitor; niacin receptor agonists such as acipimox and acifran, as well as niacin receptor partial agonists; metabolic altering agents including insulin sensitizing agents and related compounds for the treatment of diabetes such as biguanides (e.g., metformin), meglitinides (e.g., repaglinide, nateglinide), sulfonylureas (e.g., chlorpropamide, glimepiride, glipizide, glyburide, tolazamide, tolbutamide), thiazolidinediones also referred to as glitazones (e.g., pioglitazone, rosiglitazone), alpha glucosidase inhibitors (e.g., acarbose, miglitol), dipeptidyl peptidase inhibitors, (e.g., sitagliptin (JANUVIA®), alogliptin, vildagliptin, saxagliptin, linagliptin, dutogliptin, gemigliptin), ergot alkaloids (e.g., bromocriptine), combination medications such as JANUMET® (sitagliptin with metformin), and injectable diabetes medications such as exenatide and pramlintide acetate; inhibitors of glucose uptake, such as sodium-glucose transporter (SGLT) inhibitors and its various isoforms, such as SGLT-1, SGLT-2 (e.g., ASP-1941, TS-071, BI-10773, tofogliflozin, LX-4211, canagliflozin, dapagliflozin, ertugliflozin, ipragliflozin and remogliflozin), and SGLT-3; or with other drugs beneficial for the prevention or the treatment of the above-mentioned diseases including but not limited to diazoxide; and including the free-acid, free-base, and pharmaceutically acceptable salt forms, pro-drug forms, e.g., esters, and salts of pro-drugs of the above medicinal agents, where chemically possible. Trademark names of pharmaceutical drugs noted above are provided for exemplification of the marketed form of the active agent(s); such pharmaceutical drugs could be used in a separate dosage form for concurrent or sequential administration with a compound of the invention, or the active agent(s) therein could be used in a fixed dose drug combination including a compound of the invention.

Typical doses of Factor XIa inhibitors or Factor XIa/plasma kallikrein inhibitors of the invention in combination with other suitable anti-platelet agents, anticoagulation agents, or thrombolytic agents may be the same as those doses of Factor XIa inhibitors administered without coadministration of additional anti-platelet agents, anticoagulation agents, or thrombolytic agents, or may be substantially less that those doses of thrombin inhibitors administered without coadministration of additional anti-platelet agents, anticoagulation agents, or thrombolytic agents, depending on a patient's therapeutic needs.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e. prevent, inhibit or ameliorate) the thromboembolic and/or inflammatory disease condition or treat the progression of the disease in a host.

The compounds of the invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of each of the compounds when administered individually as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

The present invention is not limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

Abbreviations used herein are as follows:

Boc is tert-butyloxycarbonyl
BOP Cl is bis(2-oxo-3-oxazolidinyl)phosphinic chloride
BOP reagent is benzotriazol-1-yloxy tris(dimethylamino)phosphonium hexafluorophosphate
celite is Celite® diatomaceous earth
DCC is 1,3-dicyclohexylcarbodiimide
DCE is 1,2-dichloroethane
DCM is dichloromethane
DIPEA is diisopropylethylamine

### DMF is N,N-dimethylformamide

EDC is N-(3-dimethylaminopropyl)-N′-ethylcarbodiimide hydrochloride
HATU is O-(7-azabenzotriazol-1-yl)-N,N,N′,N′-tetramethyluronium hexafluorophosphate
HPLC is high performance liquid chromatography
LCMS is Liquid chromatography-mass spectrometry
Ph is phenyl
PyB OP is benzotriaxole-1-yl-oxy-tris-pyrrolidino-phosphonium hexafluorophosphate
SFC is supercritical fluid chromatography
S-Phos is 2-Dicyclohexylphosphino-2′,6′-dimethoxybiphenyl

### TEA is Triethanolamine

TFA is trifluoroacetic acid
THF is tetrahydrofuran
WSC HCl is 1-ethyl-3-(3′-dimethylaminopropyl)carbodiimide hydrochloride

Also, TLC is thin layer chromatography; Ts is tosyl; UV is ultraviolet; W is watts; wt. % is percentage by weight; x g is times gravity; α_{D} is the specific rotation of polarized light at 589 nm; OC is degrees Celsius; % w/v is percentage in weight of the former agent relative to the volume of the latter agent.

LCMS conditions: column: SUPELCO Ascentis Express C18 3×100 mm, 2.7 μm. Solvent system: A—0.05% TFA in water and B—0.05% TFA in Acetonitrile. Gradient condition: 10% B to 99% B in 3.5 min.

### Methods for Making the Compounds of Present Invention

### General Methods

The compounds of the present invention can be readily produced from known compounds or commercially available compounds by, for example, known processes described in published documents, and produced by production processes described below. The present invention is not limited to the production processes described below. The invention also includes processes for the preparation of compounds of the invention.

It should be noted that, when compounds of the present invention synthesized has a reactive group such as hydroxy group, amino group, carboxyl group, or thiol group as its substituent, such group may be adequately protected with a protective group in each reaction step and the protective group may be removed at an adequate stage. The process of such introduction and removal of the protective group may be adequately determined depending on the group to be protected and the type of the protective group, and such introduction and removal are conducted, for example, by the process described in the review section of Greene, T. W., et. al., “Protective Groups in Organic Synthesis”, 2007, 4th Ed., Wiley, New York, or Kocienski, P., “Protecting Groups” 1994, Thieme.

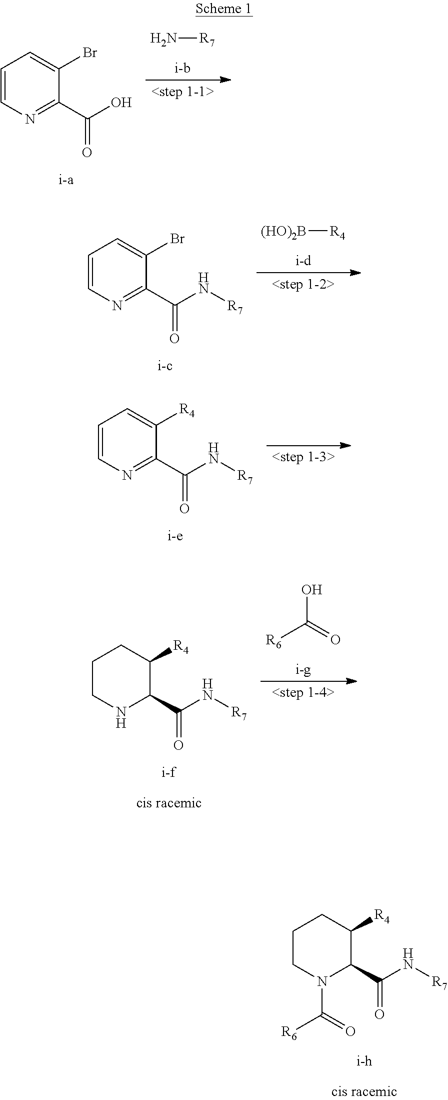

R⁴ and R⁶ are defined above. R⁷ is aryl, heteroaryl, carbocyclic, or saturated heterocyclic.

### <Step 1-1>

A compound represented by formula (i-c) can be produced by allowing 3-bromopicolinic acid (i-a) to react with a compound represented by formula (i-b) by a well-known or a process similar to that described in published documents, for example, Organic synthesis IV, Acids, amino acids, and peptides, pp. 191-309, 1992, Maruzen Co., Ltd., in the presence of a condensing agent such as 1,3-dicyclohexylcarbodiimide (DCC), 1-ethyl-3-(3′-dimethylaminopropyl)carbodiimide hydrochloride (WSC·HCl or EDC HCl), O-(7-azabenzotriazol-1-yl)-N,N,N′,N′-tetramethyluronium hexafluorophosphate (HATU), benzotriazol-1-yloxy tris(dimethylamino)phosphonium hexafluorophosphate (BOP reagent), or bis(2-oxo-3-oxazolidinyl)phosphinic chloride (BOP-Cl), in a solvent which is inactive to the reaction, such as a halogenated solvent, e.g., dichloromethane or chloroform, an ethereal solvent, e.g., diethyl ether or tetrahydrofuran, an aromatic hydrocarbon solvent, e.g., toluene or benzene, a polar solvent, e.g., N,N-dimethylformamide, or an alcoholic solvent, e.g., methanol, ethanol, or 2-propanol, in the presence or absence of a base such as triethylamine or N,N-diisopropylethyl amine at a temperature in the range of 0° C. to the solvent reflux temperature.

### <Step 1-2>

A compound represented by formula (i-e) can be produced from the reaction of a compound represented by formula (i-c) and a compound represented by (i-d) by a well-known or similar process that is described in published documents, for example, Metal Catalyzed Cross-Coupling Reactions, 2^{nd} Edition, 2004, Wiley-VCH, in the presence of a palladium catalyst such as tetrakis(triphenylphosphine)palladium(0), bis(triphenylphosphine)palladium(II) dichloride, or (1,1′-bis(diphenylphosphino)-ferrocene)palladium(II) dichloride, and inorganic base such as sodium carbonate, potassium carbonate, or potassium phosphate. The reaction can be carried out with water or without water and a solvent which is inactive to the reaction, such as toluene, N,N-dimethylformamide, dioxane, or a mixed solvent thereof at a temperature in the range of 90° C. to 120° C. using conventional or microwave heating.

### <Step 1-3>

A compound represented by formula (i-f) can be produced by allowing a compound represented by formula (i-e) to react with hydrogen gas by a well-known or similar process to that described in published documents, for example, Metal Catalyzed Reactions of Hydrocarbons, pp. 437-471, 2005, Springer US, in the presence of hydrogen gas and a catalyst such as platinum (IV) oxide, rhodium on alumina, rhodium on carbon, Raney nickel, or rhodium (III) oxide, or mixtures thereof. The reaction can be performed with or without the addition of an acid such as hydrochloric acid or acetic acid, and it can be occur in an inert solvent such as methanol, ethanol, 2-propanol, or water, or mixtures thereof, at room temperature and hydrogen pressures ranging from atmospheric pressure to 60 psi. The process as described above can generate a compound represented by formula (i-f) as a racemic mixture of cis-2,3-disubstituted piperidines.

### <Step 1-4>

A compound represented by formula (i-h) can be produced by allowing a compound represented by formula (i-f) to react with a compound of formula (i-g) by a well-known or similar process to that described in published documents, for example, Organic synthesis IV, Acids, amino acids, and peptides, pp. 191-309, 1992, Maruzen Co., Ltd., in the presence of a condensing agent such as 1,3-dicyclohexylcarbodiimide (DCC), 1-ethyl-3-(3′-dimethylaminopropyl)carbodiimide hydrochloride (WSC·HCl or EDC HCl), O-(7-azabenzotriazol-1-yl)-N,N,N′,N′-tetramethyluronium hexafluorophosphate (HATU), benzotriazol-1-yloxy tris(dimethylamino)-phosphonium hexafluorophosphate (BOP reagent), bis(2-oxo-3-oxazolidinyl)phosphinic chloride (BOP-Cl), or (benzotriazol-1-yloxy)tripyrrolidinophosphonium hexafluorophosphate (PyBOP) in a solvent which is inactive to the reaction, such as a halogenated solvent, e.g., dichloromethane or chloroform, an ethereal solvent, e.g., diethyl ether or tetrahydrofuran, an aromatic hydrocarbon solvent, e.g., toluene or benzene, a polar solvent, e.g., N,N-dimethyl-formamide, or an alcoholic solvent, e.g., methanol, ethanol, or 2-propanol, in the presence or absence of a base such as triethylamine or N,N-diisopropylethyl amine at a temperature in the range of 0° C. to the solvent reflux temperature. The process as described above can generate a compound of formula (i-h) as a racemic mixture of cis 2,3-disubstituted piperidines. A compound of formula (i-h) can be obtained as a single enantiomer using a chiral resolution process such as chiral preparatory HPLC or chiral supercritical fluid chromatography (SFC).

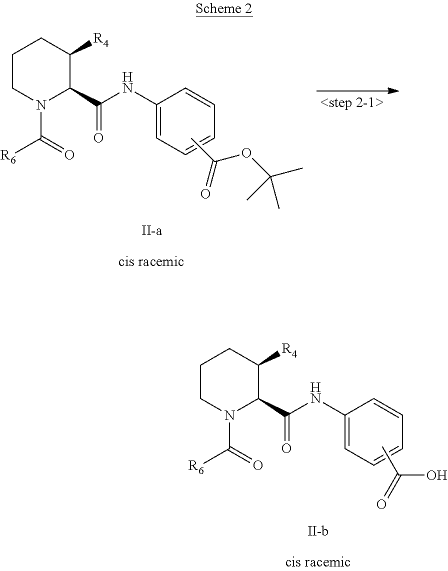

R⁴ and R⁶ are defined above.

### <Step 2-1>

In the specific case where a compound of formula (i-h) (Scheme 1) contains a tert-butyl ester group, as represented by a compound of formula (ii-a), a carboxylic acid compound represented by formula (ii-b) can be produced following a well-known process or a process similar to that described in published documents, for example, Greene, T. W., et. al., Protective Groups in Organic Synthesis (2007), 4th Ed., in the presence of an acid such as trifluoroacetic acid, formic acid, hydrochloric acid, or acetic acid in a solvent which is inactive to the reaction, such as a halogenated solvent, e.g., dichloromethane or chloroform, or an ethereal solvent, e.g., dioxane or tetrahydrofuran, at a temperature in the range of 0° C. to the solvent reflux temperature. The process as described above can generate a compound of formula (ii-b) as a racemic mixture of cis 2,3-disubstituted piperidines. A compound of formula (ii-b) can be obtained as a single enantiomer using a chiral resolution process such as chiral preparatory HPLC or chiral supercritical fluid chromatography (SFC).

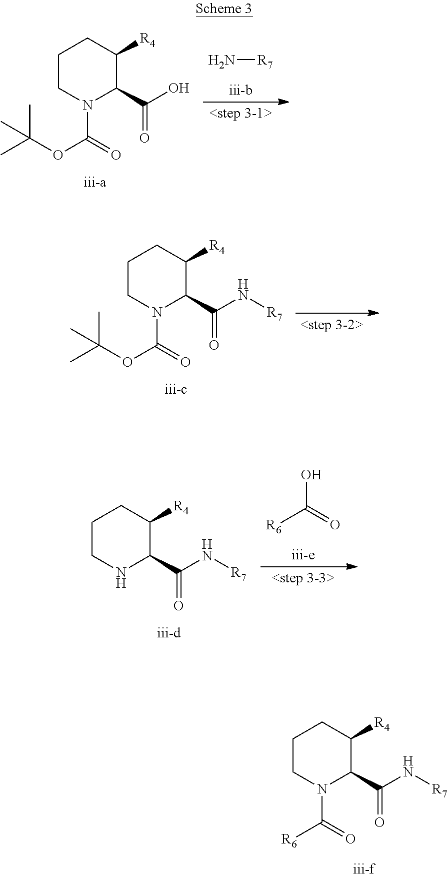

R⁴ and R⁶ are defined above. R⁷ is aryl, heteroaryl, carbocyclic, or saturated heterocyclic.

### <Step 3-1>

A compound represented by formula (iii-c) can be produced by a similar process as that used in <Step 1-1> of (Reaction Scheme 1) using a compound represented by formula (iii-a) with a compound represented by formula iii-b. In this process, the compound of formula (iii-a) can be a single enantiomer or a racemic mixture.

### <Step 3-2>

A compound represented by formula (iii-d) can be prepared by allowing a compound of formula (iii-c) to react following a well-known process or a process similar to that described in published documents, for example, Greene, T. W., et. al., Protective Groups in Organic Synthesis (2007), 4th Ed., in the presence of an acid such as trifluoroacetic acid, hydrochloric acid, or sulfuric acid in a solvent which is inactive to the reaction, such as a halogenated solvent, e.g., dichloromethane or chloroform, or an ethereal solvent, e.g., dioxane, diethyl ether, or tetrahydrofuran, or an alcohol, e.g., methanol or ethanol, at room temperature.

### <Step 3-3>

A compound represented by formula (iii-f) can be produced by the similar process as that used in <Step 1-4> of (Reaction Scheme 1) using a compound represented by formula (iii-d) with a compound represented by formula (iii-e). In the case where a compound of formula (iii-f) is a racemic mixture, a compound of formula (iii-f) can be obtained as a single enantiomer using a chiral resolution process such as chiral preparatory HPLC or chiral supercritical fluid chromatography (SFC).

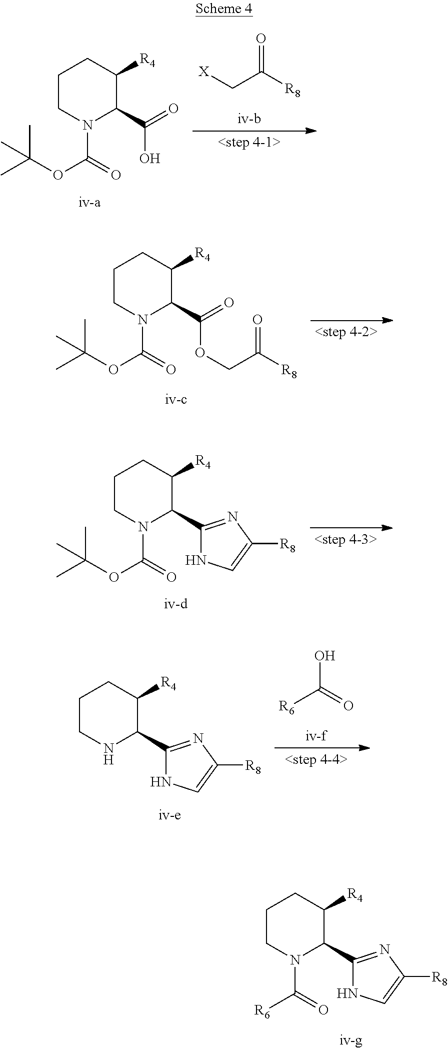

R⁴ and R⁶ are defined above. R⁸ is aryl, heteroaryl, carbocyclic, or saturated heterocyclic.

### <Step 4-1>

A compound represented by formula (iv-c) can be produced by allowing a compound of formula (iv-a) to react with a compound of formula (iv-b), where X is Cl, Br, or I, following a well-known process or a process similar to that described in published documents, for example, Contour-Galcera, M.-O., et. al., Bioorganic and Medicinal Chemistry Letters, 2001, Volume 11, Issue 5, pages 741-745, in the presence of a base such as potassium carbonate, sodium carbonate, cesium carbonate, triethylamine, or N,N-diisopropylethylamine in a solvent such as N,N-dimethylformamide, ethanol, methanol, water, or mixtures thereof, at a temperature between room temperature and 60° C. In this process, the compound of formula (iv-a) can be a single enantiomer or a racemic mixture.

### <Step 4-2>

A compound represented by formula (iv-d) can be produced by allowing a compound of formula (iv-c) to react following a well-known process or a process similar to that described in published documents, for example, Contour-Galcera, M. O., et al., Bioorganic and Medicinal Chemistry Letters, 2001, Volume 11, Issue 5, pages 741-745, in the presence of ammonium acetate in an inert solvent such as toluene, xylenes, or acetic acid, and temperatures ranging from 110° C. and 150° C. The reaction can proceed using conventional heating or microwave irradiation.

### <Step 4-3>

A compound represented by formula (iv-e) can be produced by a similar process as that used in <Step 3-2> of (Reaction Scheme 3) using a compound represented by formula (iv-d).

### <Step 4-4>

A compound represented by formula (iv-g) can be produced by a similar process as that used in <Step 1-4> of (Reaction Scheme 1) using a compound represented by formula (iv-e) with a compound represented by formula (iv-f). In the case where a compound of formula (iv-g) is a racemic mixture, a compound of formula (iv-g) can be obtained as a single enantiomer using a chiral resolution process such as chiral preparatory HPLC or chiral supercritical fluid chromatography (SFC).

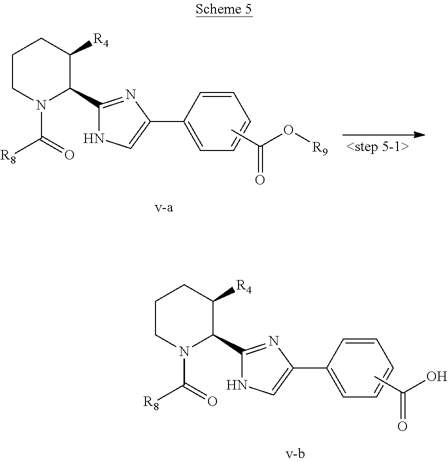

R⁴ and R⁶ are defined above. R⁹ is methyl or ethyl.

### <Step 5-1>

In the specific case where a compound of formula (iv-g) contains an ester group as represented by formula (v-a), a compound of formula (v-b) can be produced by a well-known process or a process similar to that described in published documents, for example, Greene, T. W., et. al., Protective Groups in Organic Synthesis (2007), 4th Ed., in the presence of a base such as lithium hydroxide, sodium hydroxide or potassium hydroxide and a solvent such as tetrahydrofuran, methanol, ethanol, or water or mixtures thereof, at a temperature between room temperature and the solvent reflux temperature. Alternatively, the reaction may be carried out in the presence of a Lewis acid such as boron tribromide in an inert solvent such as dichloromethane at room temperature. In the case where a compound of formula (v-b) is obtained as a racemic mixture, a compound of formula (v-b) can be obtained as a single enantiomer using a chiral resolution process such as chiral preparatory HPLC or chiral supercritical fluid chromatography (SFC).

### EXAMPLES

### Example 1-1

### (E)-tert-butyl 4-(1-(3-(5-chloro-2-(1H-tetrazol-1-yl)phenyl)acryloyl)-3-phenylpiperidine-2-carboxamido)benzoate

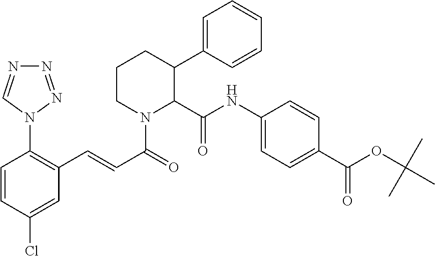 [SMILES: CC(C)(C)OC(=O)c1ccc(NC(=O)C2C(c3ccccc3)CCCN2C(=O)/C=C/c2cc(Cl)ccc2-n2cnnn2)cc1]

### Example 1-2

### (E)-1-(2-(3-(2-((4-carboxyphenyl)carbamoyl)-3-phenylpiperidin-1-yl)-3-oxoprop-1-en-1-yl)-4-chlorophenyl)-1H-tetrazol-2-ium

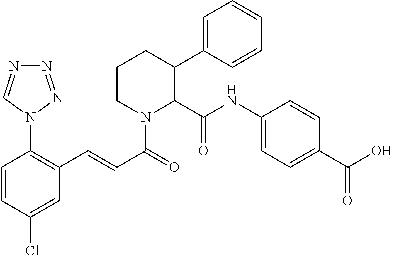 [SMILES: O=C(O)c1ccc(NC(=O)C2C(c3ccccc3)CCCN2C(=O)/C=C/c2cc(Cl)ccc2-n2cnnn2)cc1]

### Example 1-56

### 4-((2R,3R)-1-((E)-3-(5-chloro-2-(1H-tetrazol-1-yl)phenyl)acryloyl)-3-phenylpiperidine-2-carboxamido)benzoic acid

### Example 1-57

### 4-((2S,3S)-1-((E)-3-(5-chloro-2-(1H-tetrazol-1-yl)phenyl)acryloyl)-3-phenylpiperidine-2-carboxamido)benzoic acid

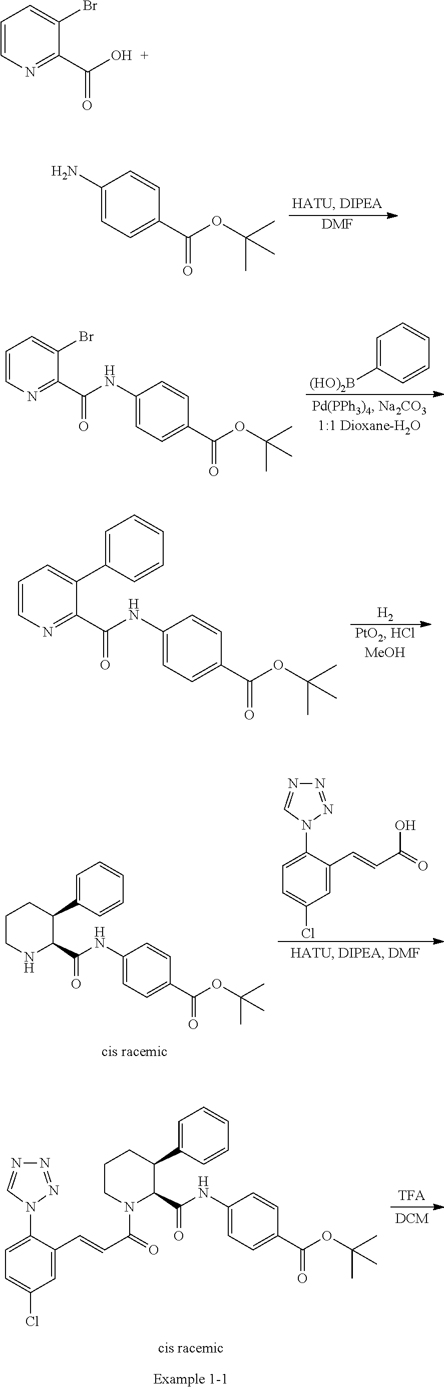

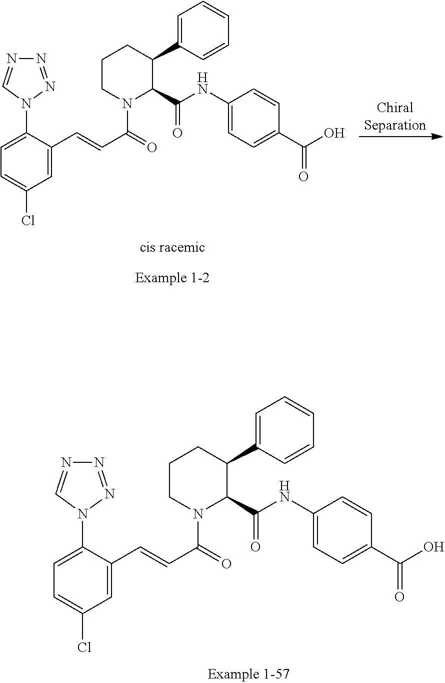 [SMILES: O=C(O)c1ccc(NC(=O)[C@@H]2[C@H](c3ccccc3)CCCN2C(=O)/C=C/c2cc(Cl)ccc2-n2cnnn2)cc1 ; O=C(O)c1ccc(NC(=O)[C@@H]2[C@H](c3ccccc3)CCCN2C(=O)/C=C/c2cc(Cl)ccc2-n2cnnn2)cc1]

### Step 1: tert-Butyl 4-(3-bromopicolinamido)benzoate

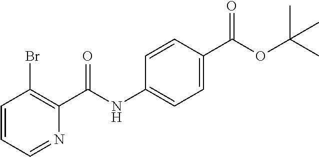 [SMILES: CC(C)(C)OC(=O)c1ccc(NC(=O)c2ncccc2Br)cc1]

N,N-Diisopropylethylamine (2.59 ml, 14.85 mmol) was added to a stirred room temperature mixture of HATU (3.76 g, 9.90 mmol), tert-butyl-4-aminobenzoate (1.15 g, 5.94 mmol), and 3-bromopicolinic acid (1 g, 5 mmol) in DMF. The mixture was stirred at room temperature overnight. The mixture was cooled and diluted with ethyl acetate. The organic phase was washed with aqueous sodium hydrogen carbonate, filtered, and the solvent was evaporated under reduced pressure. The residue was purified by silica gel column chromatography (Biotage 40M), eluting with ethyl acetate/isohexane to give tert-butyl 4-(3-bromopicolinamido)benzoate as a yellow solid. MS (ESI) m/z 378.90 (M+H).

### Step 2: tert-Butyl 4-(3-phenylpicolinamido)benzoate

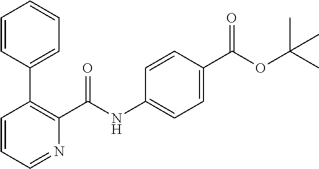 [SMILES: CC(C)(C)OC(=O)c1ccc(NC(=O)c2ncccc2-c2ccccc2)cc1]

A mixture of sodium carbonate (91 mg, 0.86 mmol), tetrakis(triphenylphosphine)palladium(0) (66.2 mg, 0.057 mmol), phenyl boronic acid (69.8 mg, 0.573 mmol), and tert-butyl 4-(3-bromopicolinamido)benzoate (108 mg, 0.286 mmol) in 1:1 dioxane-water (4 mL) was heated at 120° C. for 1 hour in a microwave reactor. The mixture was cooled, diluted with ethyl acacate and then washed with aqueous sodium hydrogen carbonate. The organic phase was dried over MgSO₄, filtered and the solvent was evaporated under reduced pressure. The residue was purified by column chromatography on silica gel (Biotage 25M), eluting with ethyl acetate/isohexane to give tert-butyl 4-(3-phenylpicolinamido)benzoate. MS (ESI) m/z 375.10 (M+H).

### Step 3: tert-Butyl 4-(3-phenylpiperidine-2-carboxamido)benzoate

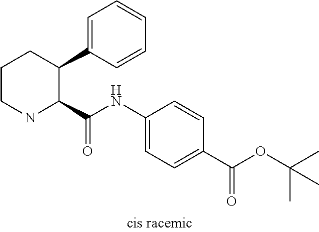 [SMILES: CC(C)(C)OC(=O)c1ccc(NC(=O)[C@H]2NCCC[C@H]2c2ccccc2)cc1]

Platinum(IV) oxide (40 mg, 0.176 mmol) was added to a stirred room temperature mixture of tert-butyl 4-(3-phenylpicolinamido)benzoate (170 mg, 0.454 mmol) and methanol (10 mL). To this mixture was added 3 N aqueous HCl (3 mL). The reaction was degassed and then stirred under a hydrogen balloon at room temperature for 2 hours. The reaction mixture was filtered through a celite pad, and aqueous sodium hydrogen carbonate was added. The filtrate was concentrated on the rotary evaporator to remove most of the methanol. The mixture was cooled, diluted with ethyl acetate and washed with aqueous sodium hydrogen carbonate. The organic phase was dried over MgSO₄, filtered and the solvent was evaporated under reduced pressure to yield tert-butyl 4-(3-phenylpiperidine-2-carboxamido)benzoate. MS (ESI) m/z 381.18 (M+H). The crude product was used in the next step without further purification.

### Step 4: (E)-tert-Butyl 4-(1-(3-(5-chloro-2-(H-tetrazol-1-yl)phenyl)acryloyl)-3-phenylpieridine-2-carboxamido)benzoate

### Example 1-1

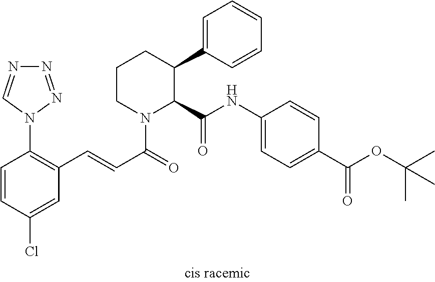 [SMILES: CC(C)(C)OC(=O)c1ccc(NC(=O)[C@@H]2[C@H](c3ccccc3)CCCN2C(=O)/C=C/c2cc(Cl)ccc2-n2cnnn2)cc1]

N,N-Diisopropylethylamine (0.213 ml, 1.222 mmol) was added to a stirred, room temperature mixture of HATU (341 mg, 0.896 mmol), tert-butyl 4-(3-phenylpiperidine-2-carboxamido)benzoate (155 mg, 0.407 mmol), and (E)-3-(5-chloro-2-(1H-tetrazol-1-yl)phenyl)acrylic acid (204 mg, 0.815 mmol) in DMF (5 mL) and the mixture was stirred at room temperature for 4 hours. The mixture was cooled and diluted with ethyl acetate. The organic phase was washed with aqueous sodium hydroxide, dried over MgSO₄, filtered and the solvent was evaporated under reduced pressure to give a yellow solid. The residue was purified by column chromatography on silica gel (Biotage 25M) eluting with ethyl acetate/isohexane to give (E)-tert-butyl 4-(1-(3-(5-chloro-2-(1H-tetrazol-1-yl)phenyl)acryloyl)-3-phenylpiperidine-2-carboxamido)benzoate MS (ESI) m/z 635.16 (M+Na).

### Step 5: (E)-1-(2-(3-(2-((4-carboxyphenyl)carbamoyl)-3-phenylpipieridin-1-yl)-3-oxoprop-1-en-1-yl)-4-chlorophenyl)-1H-tetrazol-2-ium

### Example 1-2

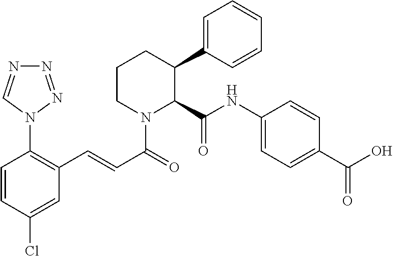 [SMILES: O=C(O)c1ccc(NC(=O)[C@@H]2[C@H](c3ccccc3)CCCN2C(=O)/C=C/c2cc(Cl)ccc2-n2cnnn2)cc1]

Trifluoroacetic acid was added to a stirred, room temperature mixture of (E)-tert-butyl 4-(1-(3-(5-chloro-2-(1H-tetrazol-1-yl)phenyl)acryloyl)-3-phenylpiperidine-2-carboxamido)benzoate in DCM and the mixture was stirred at room temperature overnight. The reaction mixture was concentrated and dissolved in MeOH and then purified by reverse phase chromatography to give the trifluoroacetic acid salt of the title compound. ¹H NMR (500 MHz, CD₃OD) δ 9.54 (s, 1H), 8.17 (s, 1H), 7.84 (d, J=8.3 Hz, 2H), 7.66-7.56 (m, 2H), 7.36-7.14 (m, 10H), 5.35 (d, J=5.4 Hz, 1H), 4.21 (d, J=11.6 Hz, 1H), 4.01 (t, J=12.9 Hz, 1H), 3.17-3.14 (m, 1H), 2.61 (q, J=11.4 Hz, 1H), 2.09 (d, J=13.8 Hz, 1H), 1.85 (d, J=13.0 Hz, 1H), 1.78 (d, J=13.2 Hz, 1H). From this product 50 mg was subjected to chiral separation using supercritical fluid chromatography (2×25 cm OJ-H column eluting with 40% methanol in CO₂ at 100 bar pressure and a flow rate of 50 mL/min) to afford 4-((2S,3S)-1-((E)-3-(5-chloro-2-(1H-tetrazol-1-yl)phenyl)acryloyl)-3-phenylpiperidine-2-carboxamido)benzoic acid (peak 1, Example 1-57). The enantiomer 4-((2R,3R)-1-((E)-3-(5-chloro-2-(1H-tetrazol-1-yl)phenyl)acryloyl)-3-phenylpiperidine-2-carboxamido)-benzoic acid (Example 1-56) was also obtained.

The following compounds may be prepared by someone skilled in the art following a procedure similar to the one described above and using appropriate starting materials.

| | | LCMS |
|---|---|---|
| Example | Structure | [M + 1]⁺ |
| 1-3 | 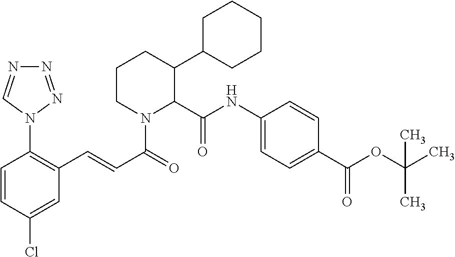 [SMILES: CC(C)(C)OC(=O)c1ccc(NC(=O)C2C(C3CCCCC3)CCCN2C(=O)/C=C/c2cc(Cl)ccc2-n2cnnn2)cc1] | 619.30 |
| 1-4 | 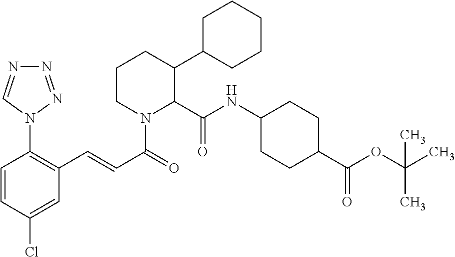 [SMILES: CC(C)(C)OC(=O)C1CCC(NC(=O)C2C(C3CCCCC3)CCCN2C(=O)/C=C/c2cc(Cl)ccc2-n2cnnn2)CC1] | 625.39 |
| 1-5 | 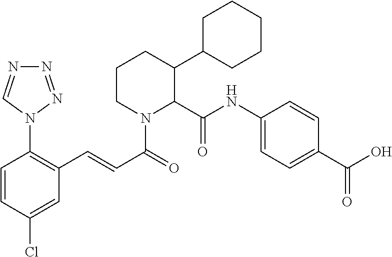 [SMILES: O=C(O)c1ccc(NC(=O)C2C(C3CCCCC3)CCCN2C(=O)/C=C/c2cc(Cl)ccc2-n2cnnn2)cc1] | 585.07 [M + Na]⁺ |
| 1-6 | 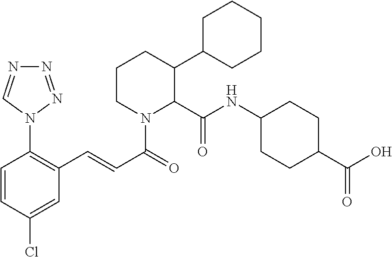 [SMILES: O=C(O)C1CCC(NC(=O)C2C(C3CCCCC3)CCCN2C(=O)/C=C/c2cc(Cl)ccc2-n2cnnn2)CC1] | 591.20 [M + Na]⁺ |
| 1-9 | 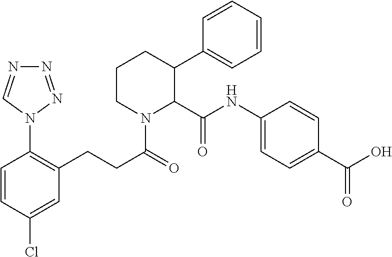 [SMILES: O=C(O)c1ccc(NC(=O)C2C(c3ccccc3)CCCN2C(=O)CCc2cc(Cl)ccc2-n2cnnn2)cc1] | 558.14 [M + Na]⁺ |
| 1-10 | 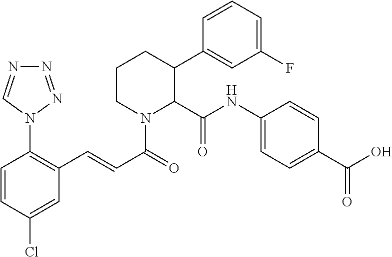 [SMILES: O=C(O)c1ccc(NC(=O)C2C(c3cccc(F)c3)CCCN2C(=O)/C=C/c2cc(Cl)ccc2-n2cnnn2)cc1] | 575.22 |
| 1-17 | 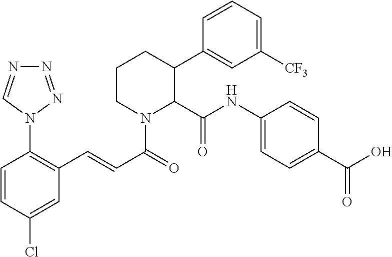 [SMILES: O=C(O)c1ccc(NC(=O)C2C(c3cccc(C(F)(F)F)c3)CCCN2C(=O)/C=C/c2cc(Cl)ccc2-n2cnnn2)cc1] | 647.22 [M + Na]⁺ |
| 1-18 | 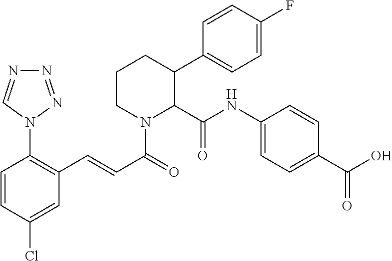 [SMILES: O=C(O)c1ccc(NC(=O)C2C(c3ccc(F)cc3)CCCN2C(=O)/C=C/c2cc(Cl)ccc2-n2cnnn2)cc1] | 597.20 [M + Na]⁺ |
| 1-19 | 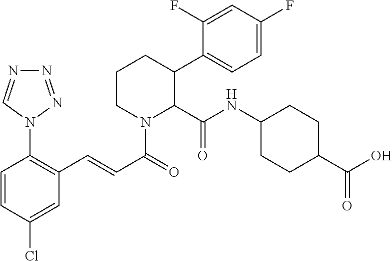 [SMILES: O=C(O)C1CCC(NC(=O)C2C(c3ccc(F)cc3F)CCCN2C(=O)/C=C/c2cc(Cl)ccc2-n2cnnn2)CC1] | 621.28 [M + Na]⁺ |
| 1-26 | 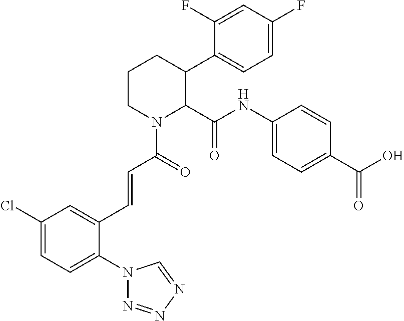 [SMILES: O=C(O)c1ccc(NC(=O)C2C(c3ccc(F)cc3F)CCCN2C(=O)/C=C/c2cc(Cl)ccc2-n2cnnn2)cc1] | 575.22 |
| 1-32 | 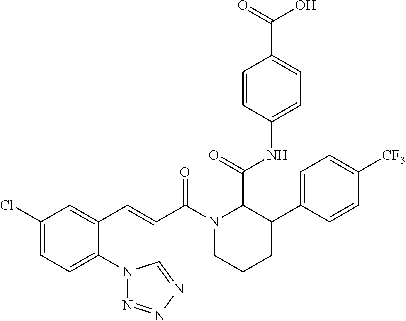 [SMILES: O=C(O)c1ccc(NC(=O)C2C(c3ccc(C(F)(F)F)cc3)CCCN2C(=O)/C=C/c2cc(Cl)ccc2-n2cnnn2)cc1] | 625.08 |
| 1-33 | 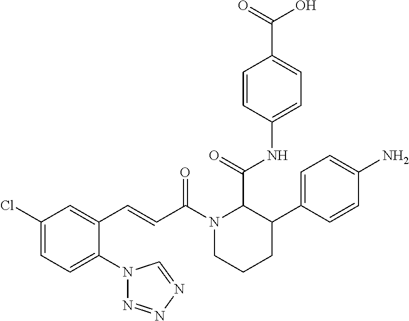 [SMILES: Nc1ccc(C2CCCN(C(=O)/C=C/c3cc(Cl)ccc3-n3cnnn3)C2C(=O)Nc2ccc(C(=O)O)cc2)cc1] | 594.21 [M + Na]⁺ |
| 1-35 | 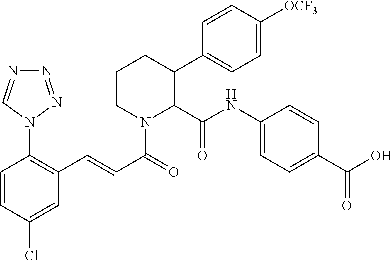 [SMILES: O=C(O)c1ccc(NC(=O)C2C(c3ccc(OC(F)(F)F)cc3)CCCN2C(=O)/C=C/c2cc(Cl)ccc2-n2cnnn2)cc1] | 663.19 [M + Na]⁺ |
| 1-36 | 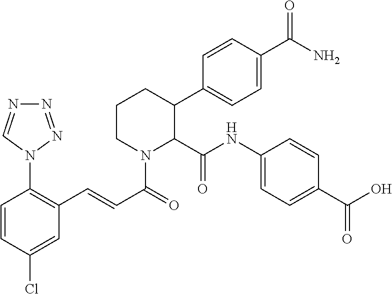 [SMILES: NC(=O)c1ccc(C2CCCN(C(=O)/C=C/c3cc(Cl)ccc3-n3cnnn3)C2C(=O)Nc2ccc(C(=O)O)cc2)cc1] | 622.33 [M + Na]⁺ |
| 1-37 | 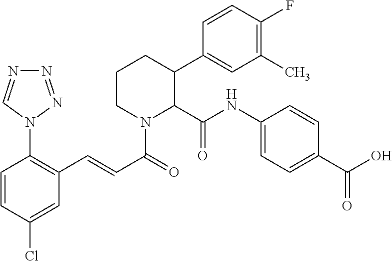 [SMILES: Cc1cc(C2CCCN(C(=O)/C=C/c3cc(Cl)ccc3-n3cnnn3)C2C(=O)Nc2ccc(C(=O)O)cc2)ccc1F] | 611.17 [M + Na]⁺ |

### Example 1-20

### (4-(2-((4-carboxyphenyl)carbamoyl)-3-phenylpiperidine-1-carbonyl)phenyl)methanaminium 2,2,2-trifluoroacetate

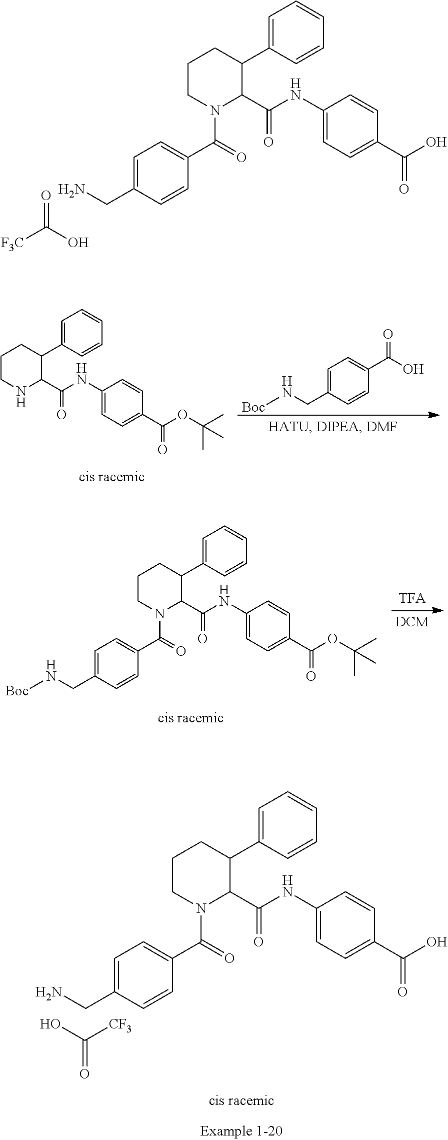

### Step 1: tert-Butyl 4-(1-(4-(((tert-butoxycarbonyl)amino)methyl)benzoyl)-3-phenylpiperidine-2-carboxamido)benzoate

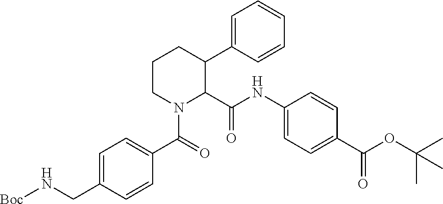 [SMILES: CC(C)(C)OC(=O)NCc1ccc(C(=O)N2CCCC(c3ccccc3)C2C(=O)Nc2ccc(C(=O)OC(C)(C)C)cc2)cc1]

DIPEA (0.069 ml, 0.394 mmol) was added to a stirred, room temperature mixture of HATU (75 mg, 0.197 mmol), 4-(((tert-butoxycarbonyl)amino)methyl)benzoic acid (33 mg, 0.131 mmol), and tert-butyl 4-(3-phenylpiperidine-2-carboxamido)-benzoate (33 mg, 0.131 mmol, prepared as described above for example 1-1) in 5 mL DCM and the mixture was stirred at room temperature overnight. The mixture was cooled and aqueous ammonium chloride was added. The mixture was extracted with ethyl acacate. The combined organic fractions were washed with brine, dried over MgSO₄, filtered and the solvent was evaporated under reduced pressure to afford the crude product which was taken on to the next step without further purification.

### Step 2: (4-(2-((4-Carboxyphenyl)carbamoyl)-3-phenylpiperidine-1-carbonyl)phenyl)methanaminium 2,2,2-trifluoroacetate

### Example 1-20

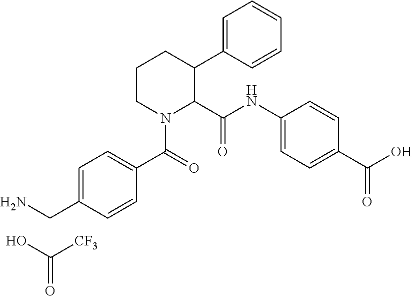

The crude material from above, tert-butyl 4-(1-(4-(((tert-butoxycarbonyl)amino)-methyl)benzoyl)-3-phenyl-piperidine-2-carboxamido)benzoate was combined with DCM (2 mL) and TFA (1 mL) and the reaction mixture was stirred at room temperature for 4 hours. After this time, the reaction mixture was concentrated, and the crude product was purified by reverse-phase HPLC to give the title compound. ¹H NMR (500 MHz, CD₃OD) δ 8.08 (s, 1H), 7.87 (d, J=8.5 Hz, 2H), 7.66-7.08 (m, 12H), 5.47 (bd, J=5 Hz, 1H), 4.20 (s, 2H), 4.03 (t, J=12.8 Hz, 1H), 3.79-3.59 (m, 1H), 2.67-2.62 (m, 1H), 2.09 (d, J=13.8 Hz, 1H), 1.94-1.60 (m, 4H).

The following compounds may be prepared by someone skilled in the art following a procedure similar to the one described above and using appropriate starting materials.

| | | LCMS |
|---|---|---|
| Example | Structure | [M + 1]⁺ |
| 1-8 | 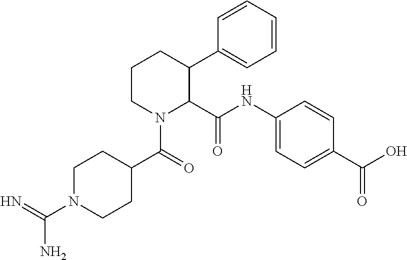 [SMILES: N=C(N)N1CCC(C(=O)N2CCCC(c3ccccc3)C2C(=O)Nc2ccc(C(=O)O)cc2)CC1] | 478.25 |
| 1-14 | 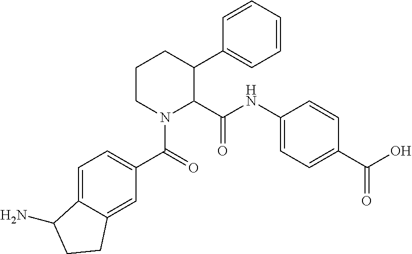 [SMILES: NC1CCc2cc(C(=O)N3CCCC(c4ccccc4)C3C(=O)Nc3ccc(C(=O)O)cc3)ccc21] | 484.28 |
| 1-39 | 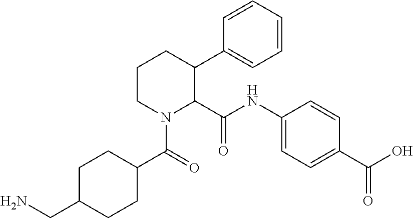 [SMILES: NCC1CCC(C(=O)N2CCCC(c3ccccc3)C2C(=O)Nc2ccc(C(=O)O)cc2)CC1] | 464.12 |
| 1-43 | 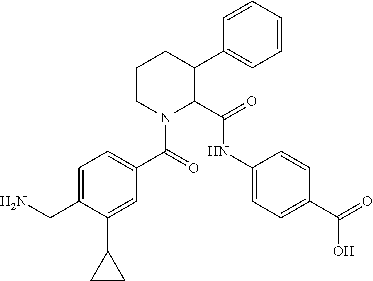 [SMILES: NCc1ccc(C(=O)N2CCCC(c3ccccc3)C2C(=O)Nc2ccc(C(=O)O)cc2)cc1C1CC1] | 497.93 |

### Example 1-44

### (E)-Methyl (3-bromo-4-(2-(1-(3-(5-chloro-2-(1H-tetrazol-1-yl)phenyl)acryloyl)-3-phenylpiperidin-2-yl)-1H-imidazol-4-yl)phenyl)carbamate

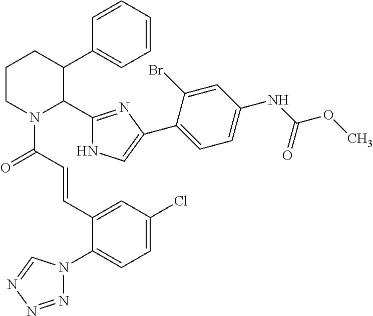 [SMILES: COC(=O)Nc1ccc(-c2c[nH]c(C3C(c4ccccc4)CCCN3C(=O)/C=C/c3cc(Cl)ccc3-n3cnnn3)n2)c(Br)c1]

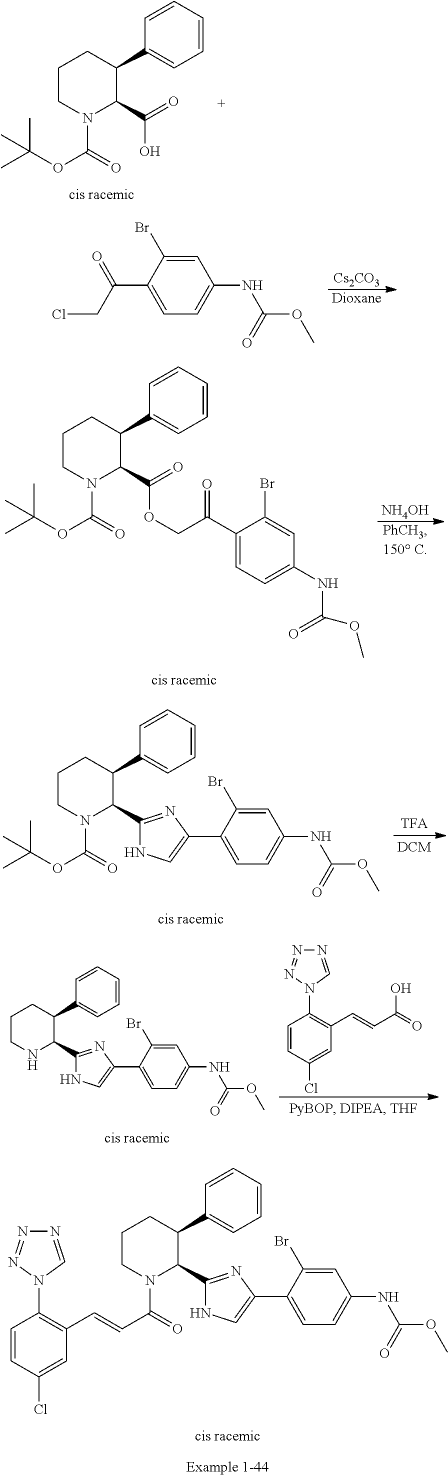

### Step 1: 2-(2-(2-Bromo-4-((methyoxycarbonyl)amino)phenyl)-2-oxoethyl 1-tert-butyl 3-phenylpiperidine-1,2-dicarboxylate

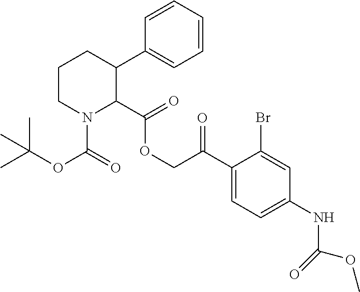 [SMILES: COC(=O)Nc1ccc(C(=O)COC(=O)C2C(c3ccccc3)CCCN2C(=O)OC(C)(C)C)c(Br)c1]

The starting material 1-(tert-butoxycarbonyl)-3-phenylpiperidine-2-carboxylic acid (0.632 g, 2.070 mmol) was dissolved in dioxane (20.0 ml). Cesium carbonate (0.877 g, 2.69 mmol) was added, followed by methyl (3-bromo-4-(2-chloroacetyl)-phenyl)carbamate (0.761 g, 2.484 mmol), and the reaction was stirred overnight at room temperature. The reaction was diluted with three volumes of dichloromethane, then filtered and washed with dichloromethane. The filtrate was concentrated to a yield a cloudy brown liquid that was resuspended in about 5 mL dichlormethane then syringe filtered to yield a homogenous red-brown solution. the solution was loaded directly on an Isco RediSep® Gold 24 g column. Flash chromatography (0-80% ethyl acetate in hexanes) was used to purify the product, giving the title compound as a viscous yellow oil. MS (ESI) m/z 597, 599 [M+Na]⁺.

### Step 2: tert-Butyl 2-(4-(2-bromo-4-((methoxycarbonyl)amino)phenyl)-1H-imidazol-2-yl)-3-phenylpiperidine-1-carboxylate

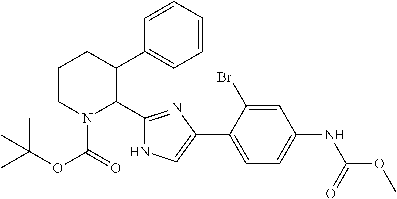 [SMILES: COC(=O)Nc1ccc(-c2c[nH]c(C3C(c4ccccc4)CCCN3C(=O)OC(C)(C)C)n2)c(Br)c1]

The starting material 2-(2-(2-bromo-4-((methoxycarbonyl)amino)phenyl)-2-oxoethyl) 1-tert-butyl 3-phenylpiperidine-1,2-dicarboxylate (0.9942 g, 1.641 mmol) was dissolved in toluene (20 mL) in a 25 mL microwave vial. Ammonium acetate (0.6100 g, 7.91 mmol) was added, and the reaction was heated using microwave irradiation for 60 minutes at 150° C. (high absorbance). The clear, light yellow solution has become a brighter yellow, and is now somewhat opaque. The reaction was resubmitted to 60 minutes irradiation at 150° C. The reaction solution was diluted with 10 mL ethyl acetate and was then washed once with 10 mL 500/% saturated cesium chloride, resulting in a bright red-orange organic layer and cloudy light yellow aqueous. The aqueous layer was extracted once with 10 mL ethyl acetate to yield an orange-yellow second organic layer that was combined with the first. The combined organic layers were washed with 8 mL of saturated cesium chloride then concentrated to yield a red-orange oil that was redissolved in dichloromethane and loaded directly on an Isco RediSep® Gold 24 g column. Flash chromatography (0-40% ethyl acetate in hexanes) afforded the title compound as a yellow solid. MS (ESI) m/z 555, 557 [M+H]⁺.

### Step 3: Methyl (3-bromo-4-(2-(3-phenylpiperidin-2-yl)-1H-imidazol-4-yl)-phenyl)carbamate

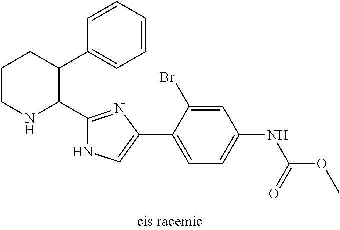 [SMILES: COC(=O)Nc1ccc(-c2c[nH]c(C3NCCCC3c3ccccc3)n2)c(Br)c1]

The starting material tert-butyl 2-(4-(2-bromo-4-((methoxycarbonyl)amino)phenyl)-1H-imidazol-2-yl)-3-phenylpiperidine-1-carboxylate (0.0794 g, 0.143 mmol) was dissolved in dichloromethane (5 ml) and TFA (1.0 ml, 12.98 mmol) and the mixture was stirred over 2 hours at room temperature. The reaction was concentrated and left under vacuum for one hour to yield the title compound as an orange oil. MS (ESI) m/z 456.93 [M+H]⁺.

### Step 4: (E)-Methyl (3-bromo-4-(2-(1-(3-(5-chloro-2-(1H-tetrazol-1-yl)-phenyl)acryloyl)-3-phenylpiperidin-2-yl)-1H-imidazol-4-yl)phenyl)carbamate

### Example 1-44

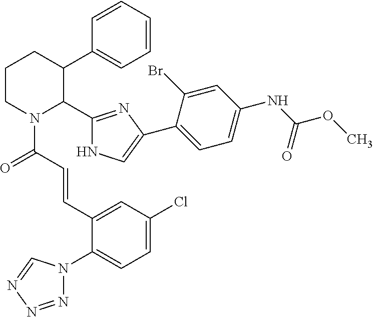 [SMILES: COC(=O)Nc1ccc(-c2c[nH]c(C3C(c4ccccc4)CCCN3C(=O)/C=C/c3cc(Cl)ccc3-n3cnnn3)n2)c(Br)c1]

Methyl (3-bromo-4-(2-(3-phenylpiperidin-2-yl)-1H-imidazol-4-yl)-phenyl)-carbamate (0.081 g, 0.143 mmol), (E)-3-(5-chloro-2-(1H-tetrazol-1-yl)phenyl)-acrylic acid_(0.054 g, 0.215 mmol), PyBOP (0.112 g, 0.215 mmol) and DIPEA (0.150 ml, 0.858 mmol) were combined in THF (6.00 ml) and the reaction mixture was stirred overnight at room temperature. The reaction was concentrated to an orange oil that was redissolved in dichloromethane and loaded on two 1000 micron Analtech prep TLC plates. The plates were run once in 75% hexane-ethyl acetate. The top band was isolated to yield the title compound as a yellow solid. MS (ESI) m/z 687, 689 [M+H]⁺.

The following compounds may be prepared by someone skilled in the art following a procedure similar to the one described above and using appropriate starting materials. In cases where a chiral, non-racemic compound is indicated, the enantiomerically pure material was either by resolution of the final product by chiral supercritical fluid chromatography or by using the chiral, non-racemic intermediate (2S,3S)-1-(tert-butoxycarbonyl)-3-phenylpiperidine-2-carboxylic acid.

| | | LCMS |
|---|---|---|
| Example | Structure | [M + 1]⁺ |
| 1-7 | 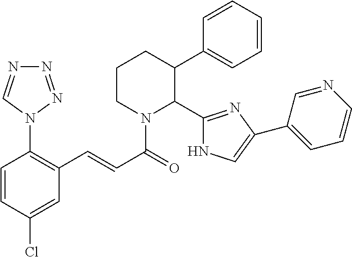 [SMILES: O=C(/C=C/c1cc(Cl)ccc1-n1cnnn1)N1CCCC(c2ccccc2)C1c1nc(-c2cccnc2)c[nH]1] | 537.21 |
| 1-13 | 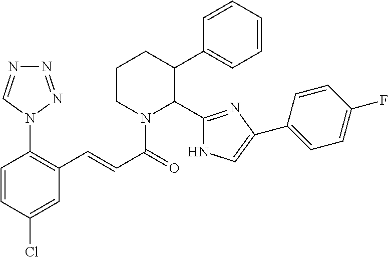 [SMILES: O=C(/C=C/c1cc(Cl)ccc1-n1cnnn1)N1CCCC(c2ccccc2)C1c1nc(-c2ccc(F)cc2)c[nH]1] | 553.88 |
| 1-21 | 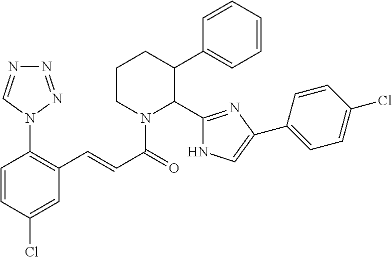 [SMILES: O=C(/C=C/c1cc(Cl)ccc1-n1cnnn1)N1CCCC(c2ccccc2)C1c1nc(-c2ccc(Cl)cc2)c[nH]1] | 570.12 |
| 1-22 | 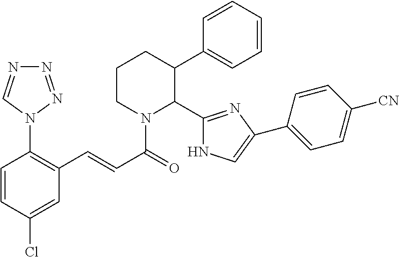 [SMILES: N#Cc1ccc(-c2c[nH]c(C3C(c4ccccc4)CCCN3C(=O)/C=C/c3cc(Cl)ccc3-n3cnnn3)n2)cc1] | 561.18 |
| 1-27 | 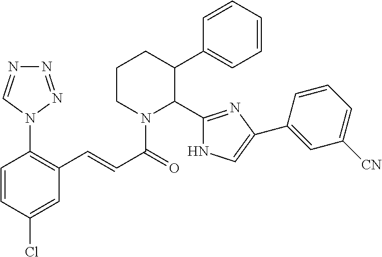 [SMILES: N#Cc1cccc(-c2c[nH]c(C3C(c4ccccc4)CCCN3C(=O)/C=C/c3cc(Cl)ccc3-n3cnnn3)n2)c1] | 560.89 |
| 1-28 | 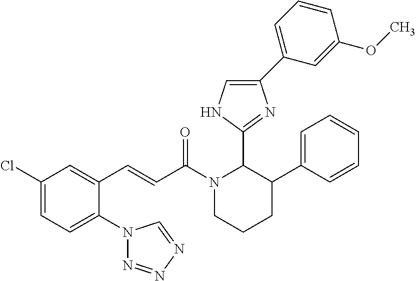 [SMILES: COc1cccc(-c2c[nH]c(C3C(c4ccccc4)CCCN3C(=O)/C=C/c3cc(Cl)ccc3-n3cnnn3)n2)c1] | 565.91 |
| 1-29 | 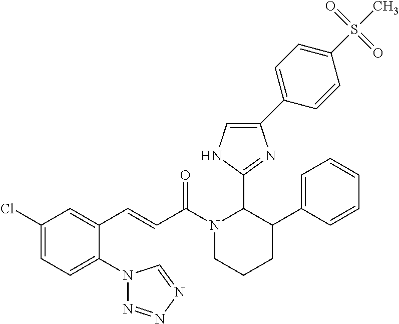 [SMILES: CS(=O)(=O)c1ccc(-c2c[nH]c(C3C(c4ccccc4)CCCN3C(=O)/C=C/c3cc(Cl)ccc3-n3cnnn3)n2)cc1] | 613.89 |
| 1-30 | 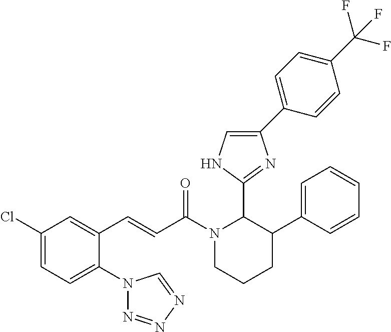 [SMILES: O=C(/C=C/c1cc(Cl)ccc1-n1cnnn1)N1CCCC(c2ccccc2)C1c1nc(-c2ccc(C(F)(F)F)cc2)c[nH]1] | 603.81 |
| 1-31 | 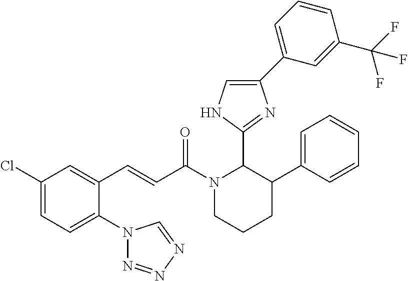 [SMILES: O=C(/C=C/c1cc(Cl)ccc1-n1cnnn1)N1CCCC(c2ccccc2)C1c1nc(-c2cccc(C(F)(F)F)c2)c[nH]1] | 603.80 |
| 1-38 | 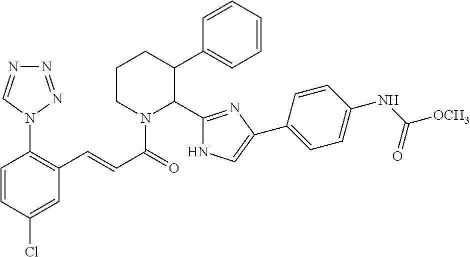 [SMILES: COC(=O)Nc1ccc(-c2c[nH]c(C3C(c4ccccc4)CCCN3C(=O)/C=C/c3cc(Cl)ccc3-n3cnnn3)n2)cc1] | 608.88 |
| 1-41 | 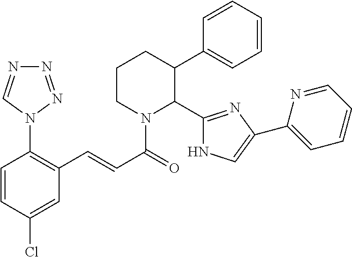 [SMILES: O=C(/C=C/c1cc(Cl)ccc1-n1cnnn1)N1CCCC(c2ccccc2)C1c1nc(-c2ccccn2)c[nH]1] | 537.09 |
| 1-42 | 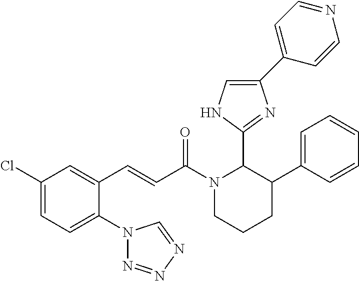 [SMILES: O=C(/C=C/c1cc(Cl)ccc1-n1cnnn1)N1CCCC(c2ccccc2)C1c1nc(-c2ccncc2)c[nH]1] | 537.05 |
| 1-46 | 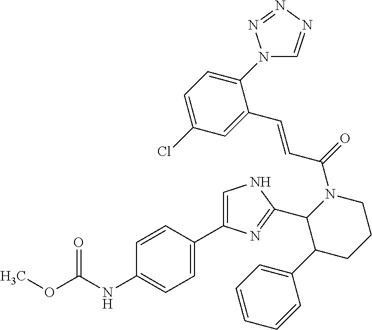 [SMILES: COC(=O)Nc1ccc(-c2c[nH]c(C3C(c4ccccc4)CCCN3C(=O)/C=C/c3cc(Cl)ccc3-n3cnnn3)n2)cc1] | 608.91 |
| 1-48 | 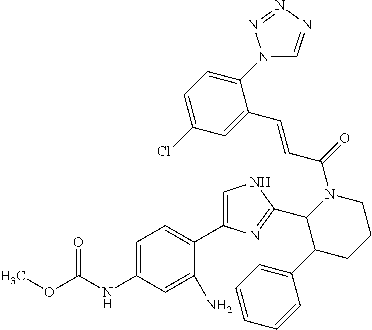 [SMILES: COC(=O)Nc1ccc(-c2c[nH]c(C3C(c4ccccc4)CCCN3C(=O)/C=C/c3cc(Cl)ccc3-n3cnnn3)n2)c(N)c1] | 623.90 |
| 1-63 | 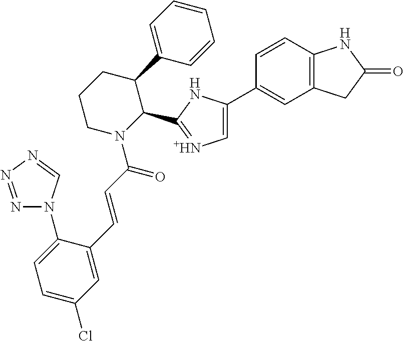 [SMILES: O=C1Cc2cc(-c3c[nH+]c([C@@H]4[C@H](c5ccccc5)CCCN4C(=O)/C=C/c4cc(Cl)ccc4-n4cnnn4)[nH]3)ccc2N1] | 595.51 |
| 1-76 | 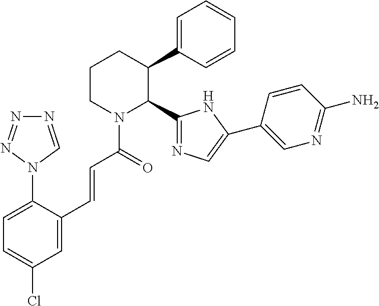 [SMILES: Nc1ccc(-c2cnc([C@@H]3[C@H](c4ccccc4)CCCN3C(=O)/C=C/c3cc(Cl)ccc3-n3cnnn3)[nH]2)cn1] | 552.27 |

### Example 1-52

### (E)-methyl (4-(2-(1-(3-(5-chloro-2-(1H-tetrazol-1-yl)phenyl)acryloyl)-3-phenylpiperidin-2-yl)-5-methyl-1H-imidazol-4-yl)phenyl)carbamate

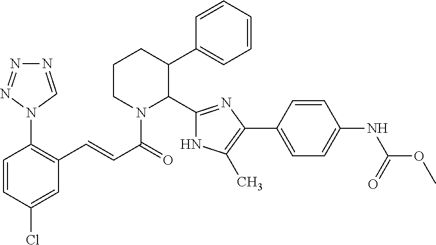 [SMILES: COC(=O)Nc1ccc(-c2nc(C3C(c4ccccc4)CCCN3C(=O)/C=C/c3cc(Cl)ccc3-n3cnnn3)[nH]c2C)cc1]

### Example 1-55

### Methyl (4-(5-chloro-2-((2S,3S)-1-((E)-3-(5-chloro-2-(1H-tetrazol-1-yl)phenyl)acryloyl)-3-phenylpiperidin-2-yl)-1H-imidazol-4-yl)phenyl)carbamate

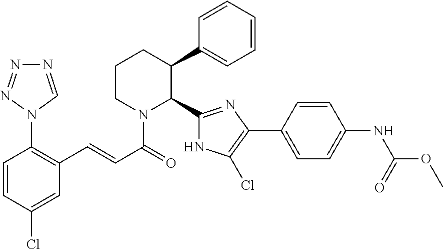 [SMILES: COC(=O)Nc1ccc(-c2nc([C@@H]3[C@H](c4ccccc4)CCCN3C(=O)/C=C/c3cc(Cl)ccc3-n3cnnn3)[nH]c2Cl)cc1]

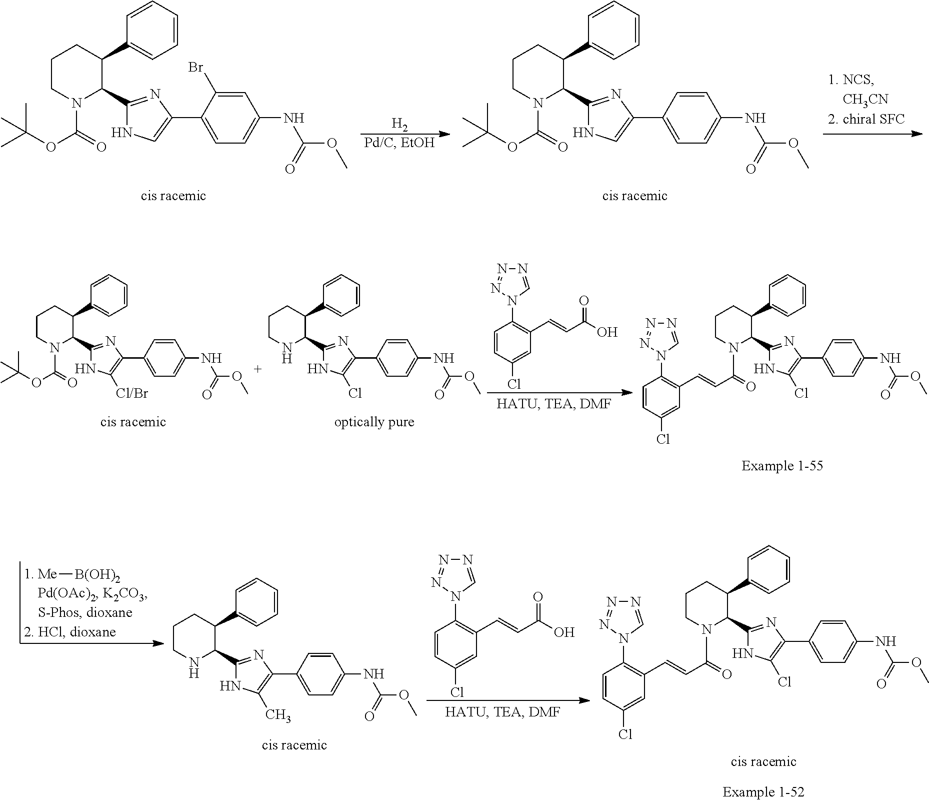

### Step 1: tert-Butyl 2-(4-(4-((methoxycarbonyl)amino)phenyl)-1H-imidazol-2-yl)-3-phenylpiperidine-1-carboxylate

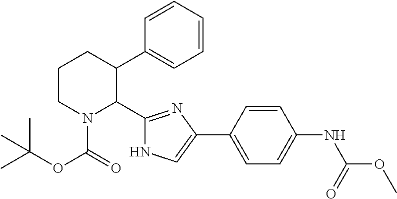 [SMILES: COC(=O)Nc1ccc(-c2c[nH]c(C3C(c4ccccc4)CCCN3C(=O)OC(C)(C)C)n2)cc1]

The starting material tert-butyl 2-(4-(2-bromo-4-((methoxycarbonyl)amino)-phenyl)-1H-imidazol-2-yl)-3-phenylpiperidine-1-carboxylate (216 mg, 0.389 mmol, described above in the preparation of Example 1-44) was combined with EtOH (10 mL) in a 250 mL round bottom flask. Palladium on carbone (20 mg, 0.188 mmol) was added, then the reaction mixture was back-filled with hydrogen gas using a balloon. The reaction mixture was stirred under a balloon of hydrogen gas for 2.5 hours. After this time, LCMS shows desired product but mostly unreacted starting material. Reaction mixture was back-filled with nitrogen than an additional 20 mg of catalyst was carefully added. Backfilled again with a balloon of hydrogen gas and the reaction mixture was stirred at room temperature under the hydrogen gas balloon for another 22 hours. The reaction mixture was evacuated to remove hydrogen then backfilled with nitrogen gas. The reaction mixture was then filtered through celite and the celite layer was washed with ethanol. The filtrate and washings were combined and concentrated to afford the title compound as a tan brittle foam. MS (ESI) m/z 477.32 (M+H).

### Step 2: Methyl (4-(5-chloro-2-((2S,3S)-3-phenylpiperidin-2-yl)-1H-imidazol-4-yl)phenyl)carbamate

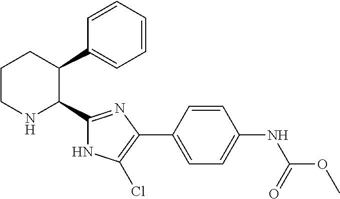 [SMILES: COC(=O)Nc1ccc(-c2nc([C@H]3NCCC[C@H]3c3ccccc3)[nH]c2Cl)cc1]

A 50 mL round bottom flask was charged with tert-butyl 2-(4-(4-((methoxy-carbonyl)amino)phenyl)-1H-imidazol-2-yl)-3-phenylpiperidine-1-carboxylate (185 mg, 0.388 mmol), 1-chloropyrrolidine-2,5-dione (55 mg, 0.412 mmol), and acetonitrile (5 mL). The reaction mixture was heated at 65° C. over 4 hours. After this time, two main product peaks appeared LCMS. One product appeared to be the title compound and the other product appeared to the boc-protected product. A brominated side product also was observed, likely from residual HBr in the last step. The reaction mixture was cooled to room temperature and then combined with 10 mL EtOAc. The organic phase was washed twice with saturated aqueous NaHCO₃. The organic phase was dried over Na₂SO₄, filtered, then concentrated to afford the crude product as an orange oil. Flash chromatography (2% MeOH-DCM ramped to 10% MeOH-DCM) was used to isolate the products. Fractions containing the title compound were collected and subjected to chiral supercritical fluid chromatography to resolve the two enantiomers of the title compound. The fastest eluting SFC peak was taken on to the next step. MS (ESI) m/z 411.25 (M+H).

### Step 3: Methyl(4-(5-chloro-2-((2S,3S)-1-((E)-3-(5-chloro-2-(H-tetrazol-1-yl)phenyl)acryloyl)-3-phenylpiperidin-2-yl)-1H-imidazol-4-yl)phenyl)carbamate

### Example 1-55

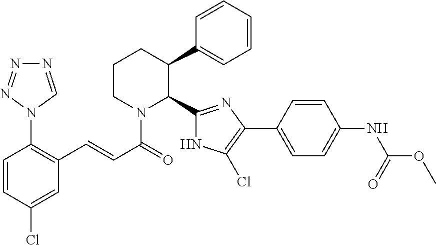 [SMILES: COC(=O)Nc1ccc(-c2nc([C@@H]3[C@H](c4ccccc4)CCCN3C(=O)/C=C/c3cc(Cl)ccc3-n3cnnn3)[nH]c2Cl)cc1]

A 20 mL vial was charged with methyl (4-(5-chloro-2-((2S,3S)-3-phenylpiperidin-2-yl)-1H-imidazol-4-yl)phenyl)carbamate (10 mg, 0.024 mmol), HATU (13.88 mg, 0.037 mmol), TEA (50 μl, 0.359 mmol), and (E)-3-(5-chloro-2-(1H-tetrazol-1-yl)phenyl)acrylic acid (9.15 mg, 0.037 mmol). DMF (500 μl) was added and the reaction mixture was stirred overnight at room temperature. In the morning, LCMS shows formation of the desired product. The reaction mixture was partioned between ethyl acetate and saturated aqueous NaHCO₃. The organic phase was filtered, concentrated and the crude product was loaded onto a 40 gram ISCO column. Flash chromatography (1.5% MeOH in DCM ramped to 5% MeOH in DCM) provided the title compound as a white solid. MS (ESI) m/z 643.38 (M+H).

### Step 4: Methyl(4-(5-methyl-2-(3-phenylpiperidin-2-yl)-1H-imidazol-4-yl)phenyl)carbamate

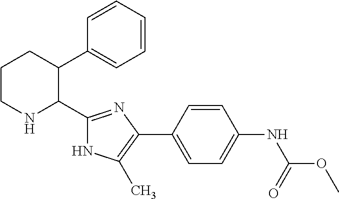 [SMILES: COC(=O)Nc1ccc(-c2nc(C3NCCCC3c3ccccc3)[nH]c2C)cc1]

A 25 mL round bottom flask was charged with tert-butyl 2-(5-bromo-4-(4-((methoxycarbonyl)amino)phenyl)-1H-imidazol-2-yl)-3-phenylpiperidine-1-carboxylate (0.080 g, 0.144 mmol), methylboronic acid (20 mg, 0.334 mmol), Palladium (II) acetate (2 mg, 8.91 μmol), S-Phos (8.24 mg, 0.017 mmol), K₂CO₃ (59.7 mg, 0.432 mmol), Dioxane (1200 μl), and Water (240 μl). The reaction mixture was heated at 90° C. for 5 hours. After this time, LCMS shows mostly the methylated product. A small amount of de-halogenation product is observed. No bromide and only trace chloride is observed. The reaction mixture was cooled to room temperature and stirred at room temperature over night. In the morning, the reaction mixture was diluted with 20 mL EtOAc, then the mixture was washed with water. The organic phase was dried over Na₂SO₄, filtered, then concentrated to an orange oil. This crude product was loaded directly onto a 24 gram ISCO column. Flash chromatography was used to isolate the boc-protected form of the title compound. MS (ESI) m/z 491.47 (M+H). This product was treated with 4 N HCl in dioxane to give the title product as a hydrochloride salt.

### Step 5: (E)-methyl (4-(2-(1-(3-(5-chloro-2-(1H-tetrazol-1-yl)phenyl)acryloyl)-3-phenylpiperidin-2-yl)-5-methyl-1H-imidazol-4-yl)phenyl)carbamate

### Example 1-55

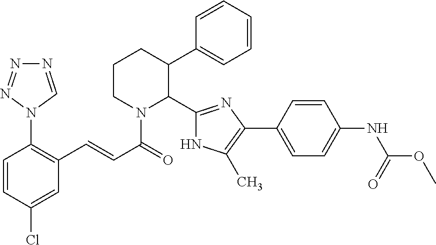 [SMILES: COC(=O)Nc1ccc(-c2nc(C3C(c4ccccc4)CCCN3C(=O)/C=C/c3cc(Cl)ccc3-n3cnnn3)[nH]c2C)cc1]

A round bottom flask was charged with (E)-3-(5-chloro-2-(1H-tetrazol-1-yl)phenyl)acrylic acid (14.68 mg, 0.059 mmol), methyl (4-(5-methyl-2-(3-phenylpiperidin-2-yl)-1H-imidazol-4-yl)phenyl)carbamate hydrochloride (25 mg, 0.059 mmol), HATU (26.7 mg, 0.070 mmol), DMF (1 ml), and TEA (0.024 ml, 0.176 mmol). The resulting mixture was stirred overnight at room temperature. In the morning, LCMS indicated the desired m/z for a dominant new product band. The reaction mixture was diluted with ethyl acetate and then the organic phase was washed with saturated aqueous sodium bicarbonate. The organic phase was dried over Na₂SO₄, filtered, then concentrated. Flash chromatography (0.5% MeOH in DCM ramped to 2% MeOH in DCM) afforded the title compound. MS (ESI) m/z 623.40 (M+H).

### Example 1-54

### (E)-methyl 3-(2-(1-(3-(5-chloro-2-(1H-tetrazol-1-yl)phenyl)acryloyl)-3-phenylpiperidin-2-yl)-1H-imidazol-4-yl)benzoate

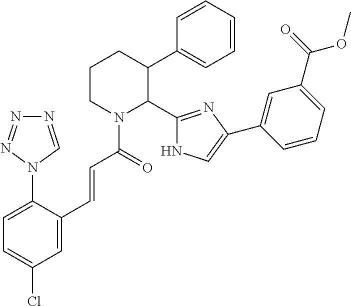 [SMILES: COC(=O)c1cccc(-c2c[nH]c(C3C(c4ccccc4)CCCN3C(=O)/C=C/c3cc(Cl)ccc3-n3cnnn3)n2)c1]

### Example 1-58

### (E)-3-(2-(1-(3-(5-chloro-2-(1H-tetrazol-1-yl)phenyl)acryloyl)-3-phenylpiperidin-2-yl)-1H-imidazol-4-yl)benzoic acid

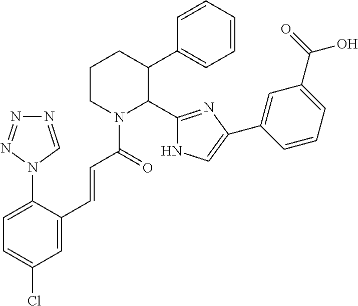 [SMILES: O=C(O)c1cccc(-c2c[nH]c(C3C(c4ccccc4)CCCN3C(=O)/C=C/c3cc(Cl)ccc3-n3cnnn3)n2)c1]

### Example 1-69

### 3-(2-((2S,3S)-1-((E)-3-(5-chloro-2-(1H-tetrazol-1-yl)phenyl)-acryloyl)-3-phenylpiperidin-2-yl)-1H-imidazol-4-yl)benzoic acid

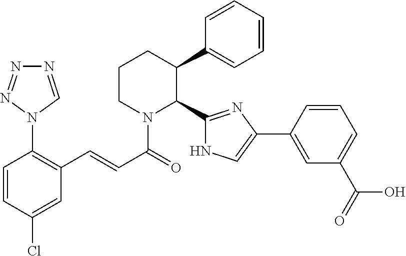 [SMILES: O=C(O)c1cccc(-c2c[nH]c([C@@H]3[C@H](c4ccccc4)CCCN3C(=O)/C=C/c3cc(Cl)ccc3-n3cnnn3)n2)c1]

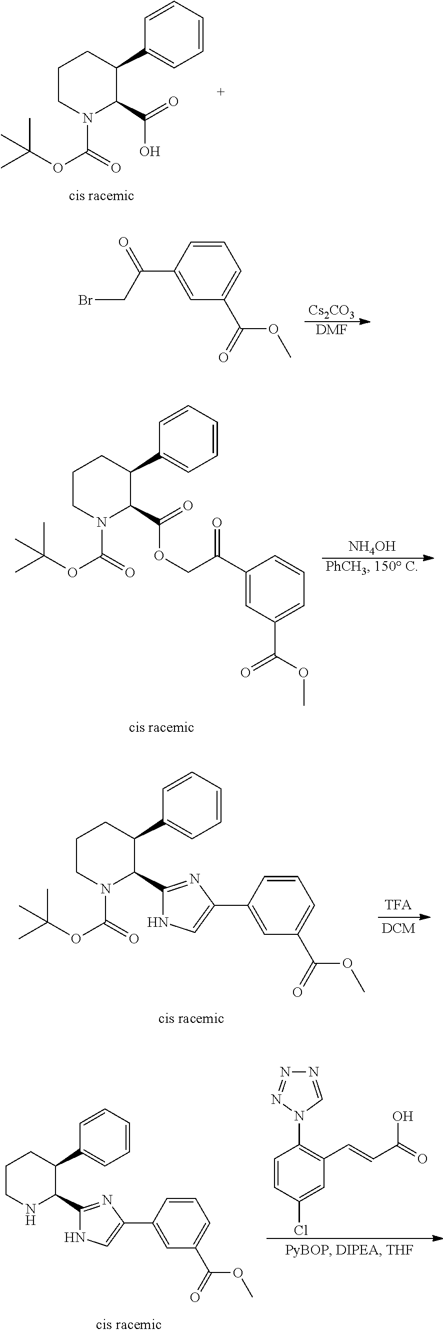

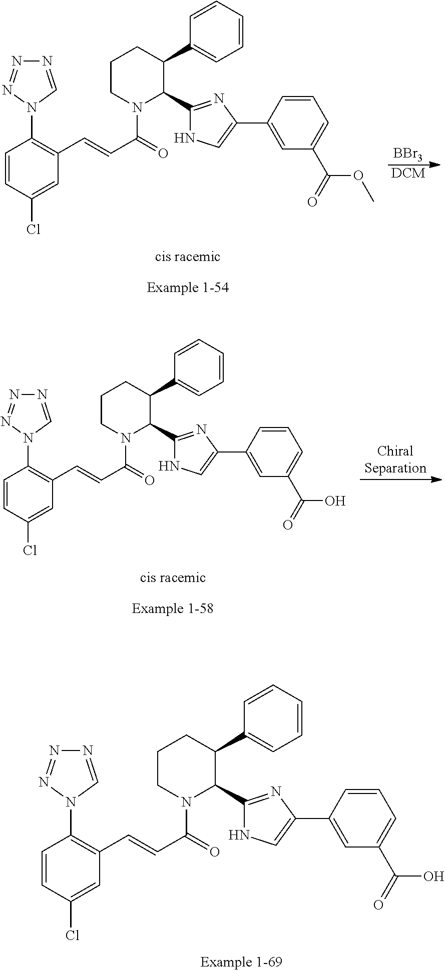

### Step 1: 1-tert-Butyl 2-(2-(3-(methoxycarbonyl)phenyl)-2-oxoethyl) 3-phenylpiperidine-1,2-dicarboxylate

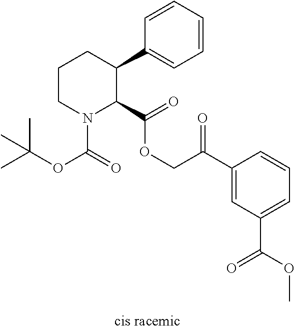 [SMILES: COC(=O)c1cccc(C(=O)COC(=O)[C@@H]2[C@H](c3ccccc3)CCCN2C(=O)OC(C)(C)C)c1]

Methyl 3-(2-bromoacetyl)benzoate (253 mg, 0.982 mmol) was added to a mixture of 1-(tert-butoxycarbonyl)-3-phenylpiperidine-2-carboxylic acid (300 mg, 0.982 mmol) and cesium carbonate (160 mg, 0.491 mmol) in DMF. The reaction mixture was stirred at room temperature over 1 hour. The mixture was diluted with ethyl acetate and washed twice with water. The organic phase was washed with brine, dried over MgSO₄, filtered and the solvent was evaporated under reduced pressure to give crude 1-tert-butyl 2-(2-(3-(methoxycarbonyl)phenyl)-2-oxoethyl) 3-phenylpiperidine-1,2-dicarboxylate (497 mg, 100%) as a colorless oil. MS (ESI) m/z 382.41 (M+H). The crude product was used in the next step without further purification.

### Step 2: tert-Butyl 2-(4-(3-(methoxycarbonyl)phenyl)-1H-imidazol-2-yl)-3-phenylpiperidine-1-carboxylate

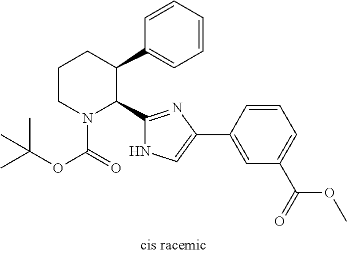 [SMILES: COC(=O)c1cccc(-c2c[nH]c([C@@H]3[C@H](c4ccccc4)CCCN3C(=O)OC(C)(C)C)n2)c1]

A microwave bottle was charged with 1-tert-butyl 2-(2-(3-(methoxycarbonyl)-phenyl)-2-oxoethyl) 3-phenylpiperidine-1,2-dicarboxylate (473 mg, 0.982 mmol), toluene (9.8 mL), and ammonium acetate (303 mg, 3.93 mmol) and then capped. The mixture was stirred at room temperature and then heated to 150° C. for 20 minutes using a microwave reactor. The reaction mixture was concentrated. The crude residue was purified by column chromatography on silica gel (ReadySep 24 g), eluting with ethyl acetate/isohexane (0-70%) to give tert-butyl-2-(4-(3-(methoxycarbonyl)phenyl)-1H-imidazol-2-yl)-3-phenylpiperidine-1-carboxylate as a light orange smear. MS (ESI) m/z 462.44 (M+H).

### Step 3: Methyl 3-(2-(3-phenylpiperidin-2-yl)-1H-imidazol-4-yl)benzoate

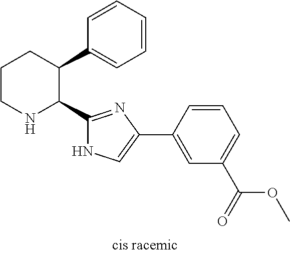 [SMILES: COC(=O)c1cccc(-c2c[nH]c([C@H]3NCCC[C@H]3c3ccccc3)n2)c1]

A mixture of tert-butyl 2-(4-(3-(methoxycarbonyl)phenyl)-1H-imidazol-2-yl)-3-phenylpiperidine-1-carboxylate (0.320 g, 0.693 mmol) was combined with DCM (6 mL) and trifluoroacetic acid (3 mL). The reaction mixture was stirred at room temperature for 1 hour and 45 minutes. The reaction mixture was concentrated to dryness to afford methyl 3-(2-(3-phenylpiperidin-2-yl)-1H-imidazol-4-yl)benzoate. MS (ESI) m/z 362.38 (M+H). The crude product was used in the next step without further purification.

### Step 4: (E)-methyl 3-(2-(1-(3-(5-chloro-2-(1H-tetrazol-1-yl)phenyl)acryloyl)-3-phenylpiperidin-2-yl)-H-imidazol-4-yl)benzoate

### Example 1-54

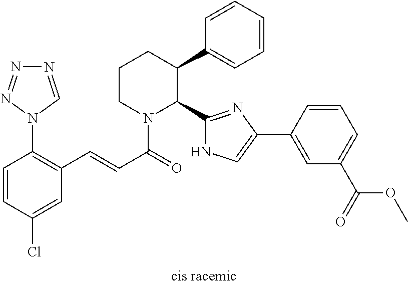 [SMILES: COC(=O)c1cccc(-c2c[nH]c([C@@H]3[C@H](c4ccccc4)CCCN3C(=O)/C=C/c3cc(Cl)ccc3-n3cnnn3)n2)c1]

Crude methyl 3-(2-(3-phenylpiperidin-2-yl)-1H-imidazol-4-yl)benzoate from above was combined with (E)-3-(5-chloro-2-(1H-tetrazol-1-yl)phenyl)acrylic acid (0.209 g, 0.832 mmol), PyBOP (0.433 g, 0.832 mmol) and THF (25 ml). DIPEA (0.727 ml, 4.16 mmol) was added, and the reaction mixture was stirred at room temperature overnight. The mixture was diluted with ethyl acetate, washed with water, brine, dried over MgSO₄, filtered and the solvent was evaporated under reduced pressure. The crude residue was purified by column chromatography on silica gel (RediSep 40 g), eluting with ethyl acetate/isohexane (30-80%) to give (E)-methyl 3-(2-(1-(3-(5-chloro-2-(1H-tetrazol-1-yl)phenyl)acryloyl)-3-phenylpiperidin-2-yl)-1H-imidazol-4-yl)benzoate as a colorless solid. MS (ESI) m/z 594.47 (M+H).

### Step 5: 3-(2-((2S,3S)-1-((E)-3-(5-chloro-2-(1H-tetrazol-1-yl)phenyl)acryloyl)-3-phenylpiperidin-2-yl)-1H-imidazol-4-yl)benzoic acid

### Example 1-58

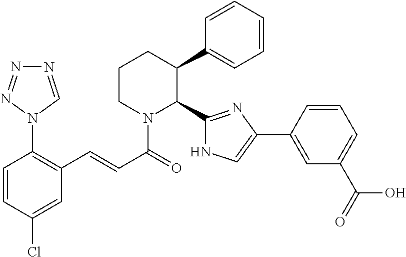 [SMILES: O=C(O)c1cccc(-c2c[nH]c([C@@H]3[C@H](c4ccccc4)CCCN3C(=O)/C=C/c3cc(Cl)ccc3-n3cnnn3)n2)c1]

Boron tribromide (0.115 ml, 1.212 mmol) was added to a cloudy solution of (E)-methyl 3-(2-(1-(3-(5-chloro-2-(1H-tetrazol-1-yl)phenyl)acryloyl)-3-phenylpiperidin-2-yl)-1H-imidazol-4-yl)benzoate (72 mg, 0.121 mmol) in DCM (1.5 mL) at room temperature. A precipitate formed instantly, and the reaction mixture was stirred at room temperature overnight. After this time, a 2:1 ratio of product to starting material was observed by UPLC. The reaction mixture was stirred at room temperature for another 24 hours. After this time, the reaction mixture was quenched carefully with aqueous NH₄Cl. Then, 2 N aqueous HCl was added and the mixture was stirred with a spatula. The solution was removed, and the slurry material was partitioned between ethyl acetate and water. The organic phase was washed with brine, dried over MgSO₄, filtered and the solvent was evaporated under reduced pressure. The crude residue was purified by preparative reverse phase HPLC (C-18 column), eluting with acetonitrile/water+0.1% TFA, to give the trifluoroacetic acid salt of (E)-3-(2-(1-(3-(5-chloro-2-(H-tetrazol-1-yl)phenyl)acryloyl)-3-phenylpiperidin-2-yl)-1H-imidazol-4-yl)benzoic acid (19.7 mg) as a colorless solid. The procedure from above was repeated using (E)-methyl 3-(2-(1-(3-(5-chloro-2-(1H-tetrazol-1-yl)phenyl)acryloyl)-3-phenylpiperidin-2-yl)-1H-imidazol-4-yl)benzoate (148 mg, 0.249 mmol) and boron tribromide (0.120 mL, 1.25 mmol) to afford additional trifluoroacetic acid salt of (E)-3-(2-(1-(3-(5-chloro-2-(1H-tetrazol-1-yl)phenyl)acryloyl)-3-phenylpiperidin-2-yl)-1H-imidazol-4-yl)benzoic acid as a colorless solid. ¹H NMR (500 MHz, CD₃OD) δ 9.53 (s, 1H), 8.19 (d, J=2.2 Hz, 1H), 8.14 (t, J=1.6 Hz, 1H), 8.05 (d, J=7.9 Hz, 1H), 7.78 (s, 1H), 7.72 (d, J=7.9 Hz, 1H), 7.68 (dd, J=8.5, 2.2 Hz, 1H), 7.57 (m, 2H), 7.36 (d, J=15.5 Hz, 1H), 7.29-7.19 (m, 3H), 7.15-7.11 (m, 3H), 6.17 (d, J=6.0 Hz, 1H), 4.41 (d, J=13.6 Hz, 1H), 3.74 (t, J=11.7 Hz, 1H), 3.55-3.49 (m, 1H), 2.39 (q, J=10.4 Hz, 1H), 2.27 (d, J=12.6 Hz, 1H), 2.10 (d, J=12.9 Hz, 1H), 1.98 (q, J=12.0 Hz, 1H). From the product above, 72.4 mg was subjected to chiral separation using supercritical fluid chromatography (21×250 mm OJ-H column eluting with 60% methanol in CO₂ at 120 bar pressure) to afford 3-(2-((2S,3S)-1-((E)-3-(5-chloro-2-(1H-tetrazol-1-yl)phenyl)acryloyl)-3-phenylpiperidin-2-yl)-1H-imidazol-4-yl)benzoic acid (peak 1, Example 1-69) as a colorless solid. MS (ESI) m/z 580.24 (M+H). The enantiomer 3-(2-((2R,3R)-1-((E)-3-(5-chloro-2-(1H-tetrazol-1-yl)phenyl)acryloyl)-3-phenylpiperidin-2-yl)-1H-imidazol-4-yl)benzoic acid (peak 2) was also obtained as a colorless solid. MS (ESI) m/z 580.23 (M+H).

The following compounds may be prepared by someone skilled in the art following a procedure similar to the one described above and using appropriate starting materials.

| | | LCMS |
|---|---|---|
| Example | Structure | [M + 1]⁺ |
| 1-40 | 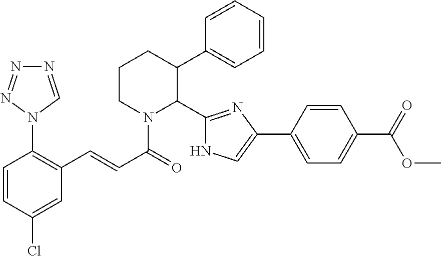 [SMILES: COC(=O)c1ccc(-c2c[nH]c(C3C(c4ccccc4)CCCN3C(=O)/C=C/c3cc(Cl)ccc3-n3cnnn3)n2)cc1] | 594.10 |
| 1-45 | 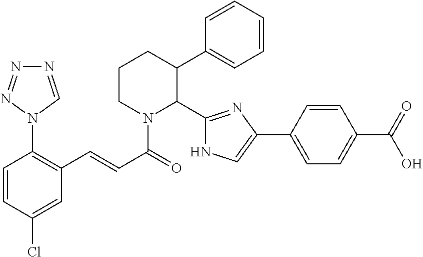 [SMILES: O=C(O)c1ccc(-c2c[nH]c(C3C(c4ccccc4)CCCN3C(=O)/C=C/c3cc(Cl)ccc3-n3cnnn3)n2)cc1] | 579.83 |

### Example 1-59

### ((E)-1-(3-(5-chloro-2-(H-tetrazol-1-yl)phenyl)acryloyl)-N-(l H-indazol-5-yl)-3-phenylpiperidine-2-carboxamide

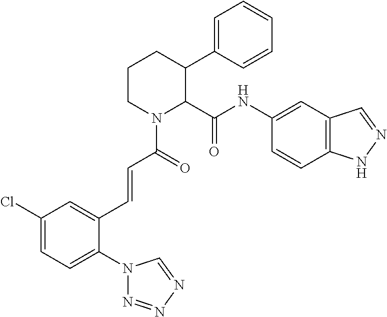 [SMILES: O=C(Nc1ccc2[nH]ncc2c1)C1C(c2ccccc2)CCCN1C(=O)/C=C/c1cc(Cl)ccc1-n1cnnn1]

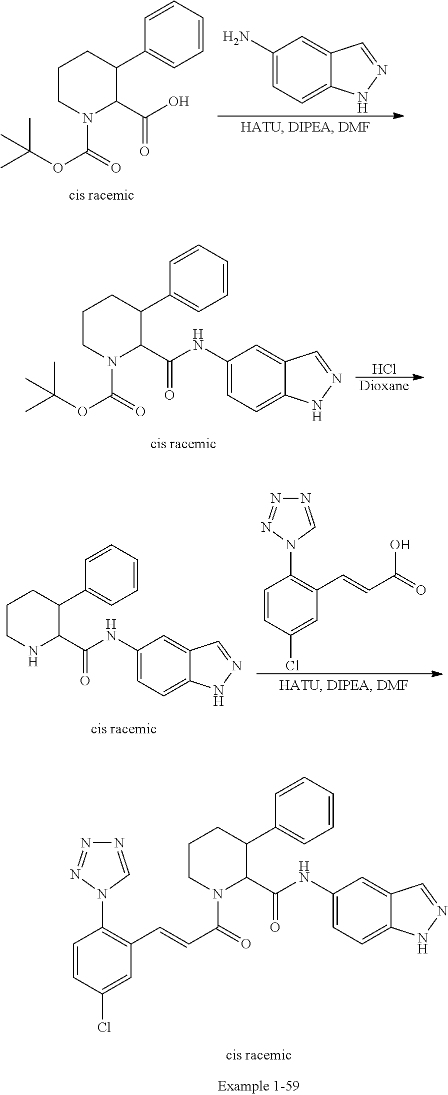

### Step 1: tert-Butyl 2-((1H-indazol-5-yl)carbamoyl)-3-phenylpiperidine-1-carboxylate

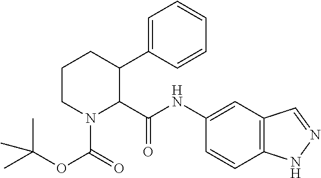 [SMILES: CC(C)(C)OC(=O)N1CCCC(c2ccccc2)C1C(=O)Nc1ccc2[nH]ncc2c1]

The starting material 1-(tert-Butoxycarbonyl)-3-phenylpiperidine-2-carboxylic acid (200 mg, 0.655 mmol), 1H-indazol-5-amine (87 mg, 0.655 mmol), and HATU (249 mg, 0.655 mmol) were dissolved in 2 mL of DMF. To this solution was added DIPEA (0.343 ml, 1.965 mmol). The brown mixture was stirred at room temperature for 2 hours then purified using flash chromatography (ISCO) (0-100° % EtOAc in hexane) to give the title compound. MS (ESI) m/z 421.47 (M+H).

### Step 2: N-(1H-indazol-5-yl)-3-phenylpiperidine-2-carboxamide

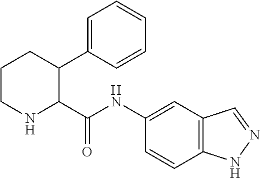 [SMILES: O=C(Nc1ccc2[nH]ncc2c1)C1NCCCC1c1ccccc1]

The crude material from above, tert-butyl 2-((1H-indazol-5-yl)carbamoyl)-3-phenylpiperidine-1-carboxylate, was dissolved in 4M HCl in dioxane, and the mixture was stirred at room temperature for 2 hours. The solvent was removed and the residue was dried under vacuum to give the title compound which was used directly in the next step. MS (ESI) m/z 321.37 (M+H).

### Step 3: ((E)-1-(3-(5-chloro-2-(1H-tetrazol-1-yl)phenyl)acryloyl)-N-(1H-indazol-5-yl)-3-phenylpiperidine-2-carboxamide

### Example 1-59

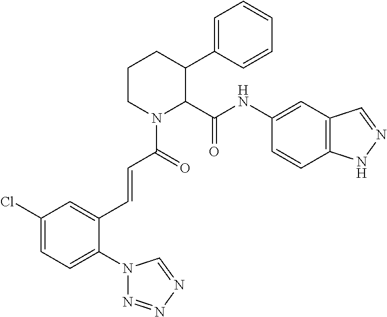 [SMILES: O=C(Nc1ccc2[nH]ncc2c1)C1C(c2ccccc2)CCCN1C(=O)/C=C/c1cc(Cl)ccc1-n1cnnn1]

To a mixture of HATU (91 mg, 0.239 mmol), N-(1H-indazol-5-yl)-3-phenylpiperidine-2-carboxamide (107 mg, 0.299 mmol), (E)-3-(5-chloro-2-(1H-tetrazol-1-yl)phenyl)acrylic acid (50 mg, 0.199 mmol) in 1.0 mL DMF was added DIPEA (0.174 ml, 0.997 mmol). The mixture was then diluted with DCM and washed with water followed by saturated aqueous sodium bicarbonate. The organic phase was dried over MgSO₄, filtered and purified on reverse phase HPLC (10-90/o MeCN in water with 0.05% TFA) to give the title compound as a TFA salt. MS (ESI) m/z 553.51 (M+H).

The following compounds may be prepared by someone skilled in the art following a procedure similar to the one described above.

| | | LCMS |
|---|---|---|
| Example | Structure | [M + 1]⁺ |
| 1-61 | 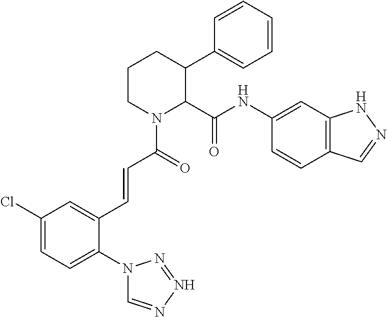 [SMILES: O=C(Nc1ccc2cn[nH]c2c1)C1C(c2ccccc2)CCCN1C(=O)/C=C/c1cc(Cl)ccc1-n1cnnn1] | 553.50 |
| 1-73 | 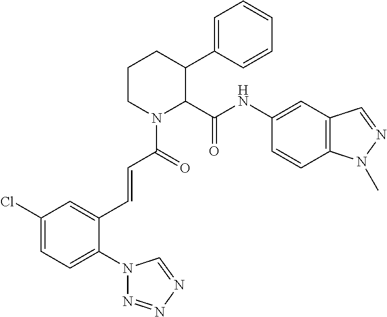 [SMILES: Cn1ncc2cc(NC(=O)C3C(c4ccccc4)CCCN3C(=O)/C=C/c3cc(Cl)ccc3-n3cnnn3)ccc21] | 567.51 |
| 1-74 | 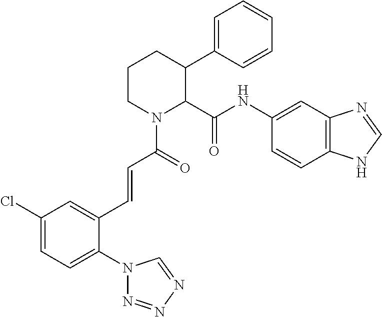 [SMILES: O=C(Nc1ccc2[nH]cnc2c1)C1C(c2ccccc2)CCCN1C(=O)/C=C/c1cc(Cl)ccc1-n1cnnn1] | 553.2 |
| 1-75 | 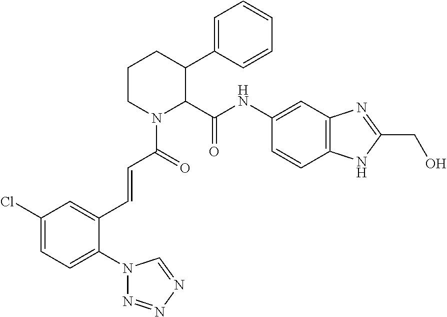 [SMILES: O=C(Nc1ccc2[nH]c(CO)nc2c1)C1C(c2ccccc2)CCCN1C(=O)/C=C/c1cc(Cl)ccc1-n1cnnn1] | 583.1 |

### INTERMEDIATES

### (E)-3-(5-chloro-2-(1H-tetrazol-1-yl)phenyl)acrylic acid

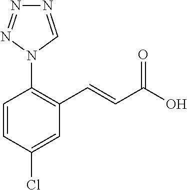 [SMILES: O=C(O)/C=C/c1cc(Cl)ccc1-n1cnnn1]

### Step 1: 5-Chloro-2-(1H-tetrazol-1-yl)benzoic acid

A suspension of 2-amino-5-chlorobenzoic acid (5.0 g, 29.1 mmol) and sodium azide (5.41 g, 83 mmol) in trimethyl orthoformate (9.12 ml, 83 mmol) was cooled to 0° C. Acetic acid (100 mL) was added, and the mixture was stirred at 0° C. for 3 hours. The reaction was warmed to room temperature and then stirred at room temperature overnight. The mixture was concentrated in vacuo, and the residue was partitioned between ethyl acetate and 3 N HCl. The organic phase was dried over MgSO₄, filtered and the solvent was evaporated under reduced pressure to give 6.28 g (100%) of 5-chloro-2-(1H-tetrazol-1-yl)benzoic acid as a light yellow solid.

### Step 2: 5-Chloro-N-methoxy-N-methyl-2-(1H-tetrazol-1-yl)benzamide

A mixture of 5-chloro-2-(l H-tetrazol-1-yl)benzoic acid (6.28 g, 28.0 mmol), PyBOP (14.55 g, 28.0 mmol), N,O-dimethylhydroxylamine hydrochloride (2.73 g, 28.0 mmol), N,N-diisopropylethylamine (14.65 mL, 84 mmol), and DCM (200 mL) was stirred at room temperature over the weekend. After this time, the reaction mixture was concentrated in vacuo. The residue was diluted with ethyl acetate, washed with water, 10% aqueous KHSO₄, saturated aqueous NaHCO₃, and brine. The organic phase was dried over MgSO₄, filtered and the solvent was evaporated under reduced pressure. The residue was purified by column chromatography on silica gel (Biotage 65i), eluting with ethyl acetate to give 6.3 g (84%) of 5-chloro-N-methoxy-N-methyl-2-(l H-tetrazol-1-yl)benzamide as an off white solid.

### Step 3: 5-Chloro-2-(1H-tetrazol-1-yl)benzaldehyde

A solution of 5-chloro-N-methoxy-N-methyl-2-(1H-tetrazol-1-yl)benzamide (2.25 g, 8.41 mmol) in 30 mL of THF was added dropwise to a stirred. −78° C. 1 M solution of LiAlH₄ (16.81 mL, 16.81 mmol) in THF. Dropwise addition occurred over a period of 30 minutes, and the mixture was stirred at −78° C. for 1 hour. After this time, 6.5 mL of cool water was added carefully. The resulting mixture was diluted with ethyl acacate, and washed with 1 M hydrochloric acid followed by brine. The organic phase was dried over MgSO₄, filtered and the solvent was evaporated under reduced pressure to give 1.56 g (89%) of 5-chloro-2-(1H-tetrazol-1-yl)benzaldehyde as a light green solid.

### Step 4: (E)-methyl 3-(5-chloro-2-(1H-tetrazol-1-yl)phenyl)acrylate

Methyl 2-(dimethoxyphosphoryl)acetate (1.402 ml, 9.72 mmol) was added dropwise to a stirred, 0° C. suspension of sodium hydride (0.359 g, 8.97 mmol) in THF (60 mL). The reaction mixture was allowed to warm up to room temperature and the stirred for 1 hour. A solution of 5-chloro-2-(1H-tetrazol-1-yl)benzaldehyde (1.56 g, 7.48 mmol) in THF (10 mL) was then added. The mixture was stirred vigorously for 30 minutes. The mixture was poured into a cold saturated NH₄Cl solution. The resulting mixture was extracted with ethyl acetate and the combined organic fractions were washed with brine, dried over MgSO₄, filtered and the solvent was evaporated under reduced pressure. The residue was recrystallized from ethyl acetate. The solid was collected and dried in vacuo to give 1.78 g (90%) of (E)-methyl 3-(5-chloro-2-(1H-tetrazol-1-yl)phenyl)acrylate as an off white solid.

### Step 5: (E)-3-(5-chloro-2-(1H-tetrazol-1-yl)phenyl)acrylic acid

A suspension of (E)-methyl 3-(5-chloro-2-(1H-tetrazol-1-yl)phenyl)acrylate (1.27 g, 4.80 mmol), 1 M aqueous sodium hydroxide (14.4 mL, 14.4 mmol) was stirred at room temperature vigorously for 2.5 hours. The mixture was neutralized with 1 N hydrochloric acid and then concentrated to give a beige solid. The solid was partitioned between 1 N hydrochloric acid and ethyl acetate. The organic layer was washed with brine, dried and concentrated to give 1.2 g (94%) of (E)-3-(5-chloro-2-(1H-tetrazol-1-yl)phenyl)acrylic acid as an off white solid. MS (ESI) m/z 251.03 (M+H), 292.11 (M+CH₃CN).

### 1-(tert-butoxycarbonyl)-3-phenylpiperidine-2-carboxylic acid

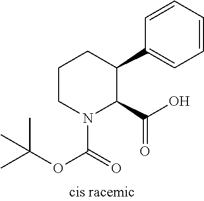 [SMILES: CC(C)(C)OC(=O)N1CCC[C@@H](c2ccccc2)[C@H]1C(=O)O]

### Step 1: 3-Phenylpiperidine-2-carboxylic acid

A solution of 3-phenyl picolinic acid (1.9949 g, 10.01 mmol) in methanol (16 mL) was added to a suspension of platinum (IV) oxide hydrate (0.1956 g, 0.798 mmol) in methanol (4 mL), and the mixture was degassed and charged with hydrogen gas from a balloon. The reaction mixture was stirred for 2.25 hours at room temperature. A sample of the reaction mixture was checked by LCMS and compared to a sample of starting material. Strong peaks for the starting material and the desired product were observed along with a weak over-reduced by-product. The hydrogen balloon was removed and the reaction was flushed with nitrogen, sealed, and left overnight. In the morning, a fresh hydrogen balloon was attached and the reaction was charged and stirred for 4.5 hours at room temperature. The balloon was removed, and the reaction mixture was diluted with dichloromethane and filtered through celite resulting in removal of a black solid. The filtrate was concentrated and left under vacuum for 30 minutes to yield 3-phenylpiperidine-2-carboxylic acid (2.0188 g, 98%) as a dark brown solid. MS (ESI) m/z 206.11 (M+H). The crude product was used in subsequent steps without further purification.

### Step 2: 1-(tert-butoxycarbonyl)-3-phenylpiperidine-2-carboxylic acid

To a solution of crude 3-phenylpiperidine-2-carboxylic acid (2.055 g, 10.01 mmol) in acetone (10 ml) and water (10.00 ml) was added sodium bicarbonate (2.52 g, 30.0 mmol) and di-tert-butyl dicarbonate (2.67 ml, 11.51 mmol). The reaction was stirred at room temperature over the weekend. Additional di-tert-butyl dicarbonate (0.711 ml, 3.06 mmol) was added, and the reaction was stirred at room temperature for 4 hours. The reaction was monitored by LCMS, indicated a mixture of starting material and desired product. Additional sodium bicarbonate (0.6200 g, 7.38 mmol) and di-tert-buty-dicarbonate (0.723 ml, 3.12 mmol) were added and the reaction was stirred overnight at room temperature. The reaction was concentrated to remove part of the acetone, and the remaining brown suspension was acidified to about pH 4 with 3.0 mL of concentrated phosphoric acid. The suspension was extracted three times with 100 mL ethyl acetate. The first extract is yellow, the second and third are colorless. The first two organic extracts were combined and dried over anhydrous sodium sulfate, filtered, washed and concentrated then chased with dichloromethane and left under vacuum for 20 minutes to yield 1-(tert-butoxycarbonyl)-3-phenylpiperidine-2-carboxylic acid (2.2440 g, 66%) as an orange-brown oil. MS (ESI) m/z 206.18 (M-C₅H₁₀O₂).

### (2S,3S)-1-(tert-butoxycarbonyl)-3-phenylpiperidine-2-carboxylic acid

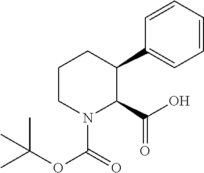 [SMILES: CC(C)(C)OC(=O)N1CCC[C@@H](c2ccccc2)[C@H]1C(=O)O]

The optically pure material (2S,3S)-1-(tert-butoxycarbonyl)-3-phenylpiperidine-2-carboxylic acid was obtained through resolution of the racemic mixture 1-(tert-butoxycarbonyl)-3-phenylpiperidine-2-carboxylic acid using chiral supercritical fluid chromatography.

### Methyl (3-bromo-4-(2-chloroacetyl)phenyl)carbamate

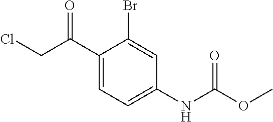 [SMILES: COC(=O)Nc1ccc(C(=O)CCl)c(Br)c1]

### Step 1: Methyl (3-bromophenyl)carbamate

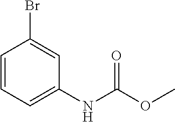

To a solution of compound 3-bromoaniline (160 g, 0.93 mol) and pyridine (95.6 g, 1.21 mol) and in anhydrous DCM (1.8 L) was added a solution of chloro-(methoxy)methanone (106.19 g, 1.12 mol) in dichloromethane (120 mL) drop-wise at 0° C. for 0.5 h, then the reaction mixture was warmed to room temperature overnight, quenched by water, extracted with DCM (800 mL), the combined organic layer was washed by water and brine, dried over anhydrous Na₂SO₄, filtered and concentrated to give a crude, which was washed with 50/200 mL of EtOAc/PE for two times to afford compound the title compound as a white solid. ¹H NMR (400 MHz, CDCl₃) δ 7.66 (s, 1H), 7.28-7.29 (d, J=3.2 Hz, 1H), 7.16-7.22 (m, 2H), 6.71 (s, 1H), 3.80 (s, 3H).

### Step 2: Methyl (3-bromo-4-(2-chloroacetyl)phenyl)carbamate

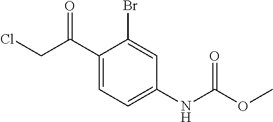 [SMILES: COC(=O)Nc1ccc(C(=O)CCl)c(Br)c1]

To a solution of methyl (3-bromophenyl)carbamate (172 g, 0.748 mol) in DCE (2 L) was added 2-chloroacethyl chloride (126.7 g, 1.121 mol) at room temperature, then cooled to 0° C., AlCl₃ (299.1 g, 2.243 mol,) in portions. The resulting solution was stirred for 2.5 h at 70° C. and cooled to room temperature, then poured into 2 L of ice water. The resulting solution was extracted with dichloromethane. The combined organic layers were washed with water and brine, dried over anhydrous Na₂SO₄, filtered and concentrated to give a crude produce, which was recrystallized from EtOAc/Pet Ether in the ratio of 1:2˜1:3 to afford the title compound as a yellow solid. ¹HNMR (400 MHz, CDCl₃) δ 7.82-7.83 (d, J=2.0 Hz, 1H), 7.53-7.55 (d, J=8.4 Hz, 1H), 7.43-7.45 (dd, J₁=J₂=2.0 Hz, 1H), 6.95 (s, 1H), 4.70 (s, 2H), 3.83 (s, 3H).

### Factor XIa Assay

The effectiveness of compound of the present invention as inhibitors of Coagulation Factor XIa can be determined using a relevant purified serine protease, and an appropriate synthetic substrate. The rate of hydrolysis of the chromogenic or fluorogenic substrate by the relevant serine protease was measured both in the absence and presence of compounds of the present invention. Assays were conducted at room temperature or at 37° C. Hydrolysis of the substrate resulted in release of amino trifluoromethylcoumarin (AFC), which was monitored spectrofluorometrically by measuring the increase in emission at 510 nm with excitation at 405 nm. A decrease in the rate of fluorescence change in the presence of inhibitor is indicative of enzyme inhibition. Such methods are known to one skilled in the art. The results of this assay are expressed as the inhibitory constant, Kᵢ.

Factor XIa determinations were made in 50 mM HEPES buffer at pH 7.4 containing 150 mM NaCl, 5 mM CaCl₂, and 0.1% PEG 8000 (polyethylene glycol; J T Baker or Fisher Scientific). Determinations were made using purified human Factor XIa at a final concentration of 40 pM (Sekisui Diagnostics) and he synthetic substrate, Z-Gly-Pro-Arg-AFC, TFA salt (Sigma #C0980) at a concentration of 100 μM.

Activity assays were performed by diluting a stock solution of substrate at least tenfold to a final concentration ≦0.1 Kₘ into a solution containing enzyme or enzyme equilibrated with inhibitor. Times required to achieve equilibration between enzyme and inhibitor were determined in control experiments. Initial velocities of product formation in the absence (Vₒ) or presence of inhibitor (Vᵢ) were measured. Assuming competitive inhibition, and that unity is negligible compared Kₘ/[S], [I]/e, and [I]/e (where [S], [I], and e respectively represent the total concentrations, of substrate, inhibitor and enzyme), the equilibrium constant (Kᵢ) for dissociation of the inhibitor from the enzyme can be obtained from the dependence of Vₒ/Vᵢ on [I] shown in the following equation.

Vₒ/Vᵢ=1+[I]/Kᵢ

The activities shown by this assay indicate that the compounds of the invention may be therapeutically useful for treating or preventing various cardiovascular and/or cerebrovascular thromboembolic conditions in patients suffering from unstable angina, acute coronary syndrome, refractory angina, myocardial infarction, transient ischemic attacks, atrial fibrillation, stroke such as thrombotic stroke or embolic stroke, venous thrombosis, coronary and cerebral arterial thrombosis, cerebral and pulmonary embolism, atherosclerosis, deep vein thrombosis, disseminated intravascular coagulation, and reocclusion or restenosis of recanalized vessels.

Factor XIa inhibition

| Example | hFXIa Ki (nM) |
|---|---|
| 1-1 | >10000 |
| 1-2 | 80.9 |
| 1-3 | >10000 |
| 1-4 | >10000 |
| 1-5 | 44 |
| 1-6 | 5252 |
| 1-7 | 904.6 |
| 1-8 | 15.42 |
| 1-9 | 57.1 |
| 1-10 | 18.11 |
| 1-11 | 8646 |
| 1-12 | 570.2 |
| 1-13 | >10000 |
| 1-14 | 572.1 |
| 1-15 | >10000 |
| 1-16 | 619.4 |
| 1-17 | 2849 |
| 1-18 | 25.32 |
| 1-19 | >10000 |
| 1-20 | 627.1 |
| 1-21 | >10000 |
| 1-22 | 3265 |
| 1-23 | >10000 |
| 1-24 | 8510 |
| 1-25 | — |
| — | — |
| — | — |
| 1-26 | — |
| | — |
| 1-27 | >10000 |
| 1-28 | >10000 |
| 1-29 | >10000 |
| 1-30 | >10000 |
| 1-31 | >10000 |
| 1-32 | 218.5 |
| 1-33 | 46.7 |
| 1-34 | 24.87 |
| 1-35 | 122.1 |
| 1-36 | 36.21 |
| 1-37 | 51.5 |
| 1-38 | 90.02 |
| 1-39 | 45.64 |
| 1-40 | >10000 |
| 1-41 | 3266 |
| 1-42 | 237.3 |
| 1-43 | 169.3 |
| 1-44 | 411.4 |
| 1-45 | 2819 |
| 1-46 | 65.47 |
| 1-47 | >10000 |
| 1-48 | 103.3 |
| 1-49 | 916 |
| 1-50 | 72.78 |
| 1-51 | >10000 |
| 1-52 | 95.77 |
| 1-53 | >10000 |
| 1-54 | >10000 |
| 1-55 | 31.54 |
| 1-56 | 4011 |
| 1-57 | 7.6 |
| 1-58 | 56.86 |
| 1-59 | 13.85 |
| 1-60 | 145.1 |
| 1-61 | 91.64 |
| 1-62 | >10000 |
| 1-63 | 206.8 |
| 1-64 | >10000 |
| 1-65 | 31.4 |
| 1-66 | 3910 |
| 1-67 | 2643 |
| 1-68 | 427.8 |
| 1-69 | 19.3 |
| 1-70 | 5735 |
| 1-71 | 2613 |
| 1-72 | 63.6 |

1. A compound of Formula I 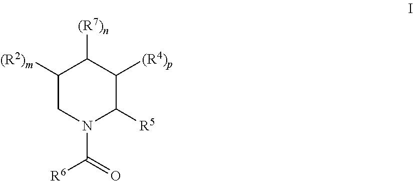
or a pharmaceutically acceptable salt thereof, wherein
m is 0 or 1, n is 0 or 1, and p is 0 or 1, provided that m+n+p=1;
R² and R⁷ are independently selected from aryl, heterocyclyl, and C₃₋₆cycloalkyl;
R⁶ is
1) —CH═CH-aryl, wherein aryl is mono or disubstituted with a substituent independently selected from the group consisting of halogen and tetrazole,
2) —CH₂CH₂-aryl, wherein aryl is mono or disubstituted with a substituent independently selected from the group consisting of halogen and tetrazole,
3) 4-7 membered monocyclic heterocyclyl having one or two heteroatoms independently selected from N, O and S, which is unsubstituted or substituted at the nitrogen atom with —C(NH)NH₂,
4) 5-7 membered monocyclic cycloalkyl or aryl, wherein cycloalkyl or aryl is unsubstituted or substituted with one or two of —CH₂NH₂, NH₂, C(CH₃)₂NH₂, C₁₋₆alkyl, or C₃₋₈ cycloalkyl,
5) 7-9 membered bicyclic aryl, which is unsubstituted or substituted with NH₂,
or
6) 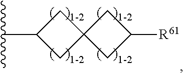
wherein R⁶¹ is —(CH₂)₀₋₁NH₂;
R⁴ is
1) —CH₂OR⁴¹, wherein R⁴¹ is hydrogen or —Si(C₁₋₆alkyl)(C₁₋₆ alkyl)(C₁₋₆ alkyl),
2) aryl, or
3) 3-7 membered monocyclic cycloalkyl,
wherein aryl is unsubstituted or substituted with one or two substituents independently selected from CF₃, halogen, NH₂, OCF₃, C(O)NH₂, C₁₋₆alkyl or 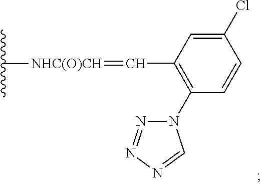
R⁵ is
1) —C(O)NHR⁵¹ wherein
R⁵¹ is
a) 3-7 membered monocyclic aryl or cycloalkyl,
b) 8 membered bicyclic cycloalkyl, or
c) 9 membered bicyclic heteroaryl,
wherein aryl or cycloalkyl is unsubstituted or substituted with one or two substituents independently selected from C(O)OC(C₁₋₆ alkyl)(C₁₋₆ alkyl)(C₁₋₆ alkyl) or C(O)OH, and wherein heteroaryl is unsubstituted or substituted with methyl or CHOH,
2) 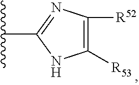
wherein
R⁵² is
a) 3-7 membered monocyclic aryl,
b) 6 membered heteroaryl containing 1 nitrogen atom, or
c) 9 membered bicyclic heteroaryl containing 1 or 2 heteroatoms, selected from N and O
wherein aryl is unsubstituted or substituted with with one or two substituents independently selected from CN, halogen, OC₁₋₆alkyl, SO₂C₁₋₆ alkyl, CF₃, C(O)OC₁₋₆ alkyl, NH₂, NHC(O)OC₁₋₆ alkyl, NHC(O)C₁₋₆ alkyl, C(O)OC(CH₃)₂, C(O)OH, PO₃H₂, or PO₃(C₁₋₆ alkyl)₂, wherein heteroaryl is unsubstituted or substituted with methyl or NH₂, and wherein bicyclic heteroaryl is unsubstituted or substituted with a ═O, and
R⁵³ is hydrogen, halogen or C₁₋₆alkyl, and
3) 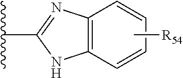
wherein
R⁵⁴ is hydrogen or halogen.

2. A compound of claim 1, or a pharmaceutically acceptable salt thereof, wherein m is 0, n is 0, p is 1,
R⁶ is
1) —CH═CH-aryl, wherein aryl is mono or disubstituted with a substituent independently selected from the group consisting of halogen and tetrazole,
2) —CH₂CH₂-aryl, wherein aryl is mono or disubstituted with a substituent independently selected from the group consisting of halogen and tetrazole,
3) 4-7 membered monocyclic heterocyclyl having one or two heteroatoms independently selected from N, O and S, which is unsubstituted or substituted at the nitrogen atom with —C(NH)NH₂,
4) 5-7 membered monocyclic cycloalkyl or aryl, wherein cycloalkyl or aryl is unsubstituted or substituted with one or two of —CH₂NH₂, NH₂, C(CH₃)₂NH₂, C₁₋₆alkyl, or C₃₋₈ cycloalkyl,
5) 7-9 membered bicyclic aryl, which is unsubstituted or substituted with NH₂,
or
6) 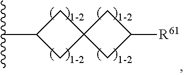
wherein R⁶ is —(CH₂)₀₋₁NH₂;
R⁴ is
1) —CH₂OR⁴¹, wherein R⁴¹ is hydrogen or —Si(C₁₋₆ alkyl)(C₁₋₆ alkyl)(C₁₋₆ alkyl),
2) aryl, or
3) 3-7 membered monocyclic cycloalkyl,
wherein aryl is unsubstituted or substituted with one or two substituents independently selected from CF₃, halogen, NH₂, OCF₃, C(O)NH₂, C₁₋₆ alkyl or 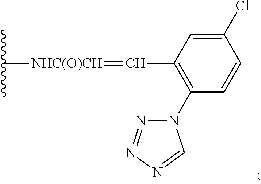
R⁵ is
1) —C(O)NHR⁵¹ wherein
R⁵¹ is
a) 3-7 membered monocyclic aryl or cycloalkyl,
b) 8 membered bicyclic cycloalkyl, or
c) 9 membered bicyclic heteroaryl,
wherein aryl or cycloalkyl is unsubstituted or substituted with with one or two substituents independently selected from C(O)OC(C₁₋₆ alkyl)(C₁₋₆ alkyl) or C(O)OH, and wherein heteroaryl is unsubstituted or substituted with methyl or CHOH,
2) 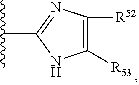
wherein
R⁵² is
a) 3-7 membered monocyclic aryl,
b) 6 membered heteroaryl containing 1 nitrogen atom, or
c) 9 membered bicyclic heteroaryl containing 1 or 2 heteroatoms, selected from N and O,
wherein aryl is unsubstituted or substituted with one or two substituents independently selected from CN, halogen, OC₁₋₆ alkyl, SO₂C₁₋₆ alkyl, CF₃, C(O)OC₁₋₆ alkyl, NH₂, NHC(O)OC₁₋₆ alkyl, NHC(O)C₁₋₆ alkyl, C(O)OC(CH₃)₂, C(O)OH, PO₃H₂, and PO₃(C₁₋₆ alkyl)₂, wherein heteroaryl is unsubstituted or substituted with methyl or NH₂, and wherein bicyclic heteroaryl is unsubstituted or substituted with ═O, and
R⁵³ is hydrogen, halogen or C₁₋₆ alkyl, or
3) 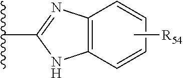
wherein
R⁵⁴ is hydrogen or halogen.

3. A compound of claim 1, or a pharmaceutically acceptable salt thereof, wherein m is 0, n is 0, p is 1, and R⁴ is
1) —CH₂OR⁴¹, wherein R⁴¹ is hydrogen or —Si(C₁₋₆ alkyl)(C₁₋₆ alkyl)(C₁₋₆ alkyl),
2) aryl, or
3) 5-7 membered monocyclic cycloalkyl,
wherein aryl is unsubstituted or substituted with one or two substituents independently selected from CF₃, halogen, NH₂, OCF₃, C(O)NH₂, C₁₋₆ alkyl or

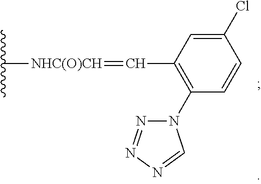

4. A compound of claim 1, or a pharmaceutically acceptable salt thereof, wherein R⁶ is
1) —CH═CH-aryl, wherein aryl is substituted with chloro and substituted with tetrazole,
2) —CH₂CH₂-aryl, wherein aryl is mono or disubstituted with a substituent independently selected from the group consisting of halogen and tetrazole,
3) 4-7 membered heterocyclyl having one N atom, wherein heterocyclyl is unsubstituted or substituted at the nitrogen atom with —C(NH)NH₂,
4) 5-7 membered monocyclic aryl, wherein aryl is unsubstituted or substituted with one or two substituents independently selected from —CH₂NH₂, NH₂, C(CH₃)₂NH₂, C(CH₃)₃, or cyclopropyl or
5) 7-9 membered bicyclic aryl, wherein bicyclic aryl is unsubstituted or substituted with NH₂.

5. A compound of claim 1, or a pharmaceutically acceptable salt thereof, wherein m is 0, n is 0, p is 1, and R⁶ is

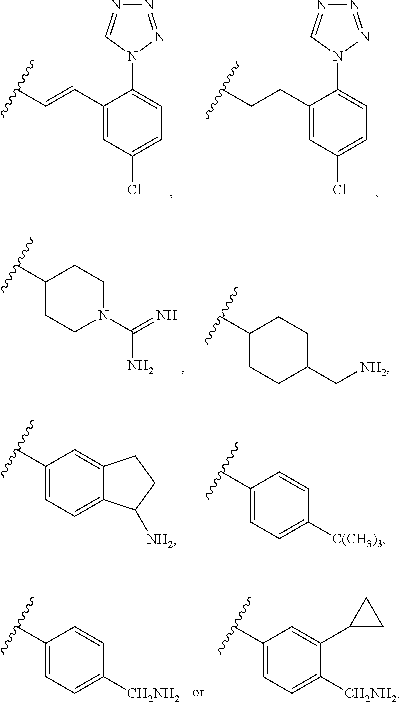

6. A compound of claim 1, or a pharmaceutically acceptable salt thereof, wherein m is 0, n is 0, p is 1 and R⁴ is
1) —CH₂OR⁴¹, wherein R⁴¹ is hydrogen or —Si(CH₃)₂(C(CH₃)₃),
2) phenyl, or
3) 5-7 membered monocyclic cycloalkyl,
wherein phenyl is unsubstituted or substituted with with one or two substituents independently selected from CF₃, F, NH₂, OCF₃, C(O)NH₂, methyl, or

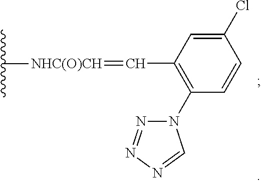

7. A compound of claim 1, or a pharmaceutically acceptable salt thereof, wherein m is 0, n is 0, p is 1, and p is 1 and R⁴ is

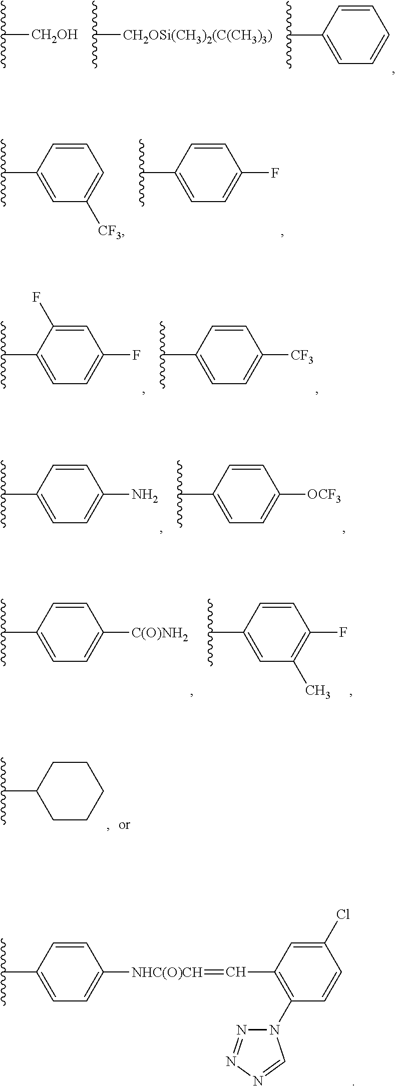

8. A compound of claim 1, or a pharmaceutically acceptable salt thereof, wherein m is 0, n is 0, p is 1, and R⁵ is
1) —C(O)NHR⁵¹, wherein R⁵¹ is
a) 5-7 membered monocyclic aryl or cycloalkyl,
b) 8 membered bicyclic cycloalkyl, or
c) 9 membered bicyclic heteroaryl,
wherein alkyl or cycloalkyl is unsubstituted or substituted with one or two substituents independently selected from C(O)OC(CH₃)₂ or C(O)OH, and wherein heteroaryl is unsubstituted or substituted with methyl or CHOH,
2) 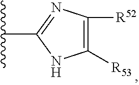
wherein
R⁵² is
a) 5-7 membered monocyclic aryl,
b) 6 membered heterocyclyl containing 1 nitrogen atom, or
c) 9 membered bicyclic heteroaryl containing 1 or 2 heteroatoms selected from N and O,
wherein aryl is unsubstituted or substituted with one or two substituents independently selected from CN, F, Cl, Br, OCH₃, SO₂CH₃, CF₃, C(O)OCH₃, NH₂, NHC(O)OCH₃, NHC(O)CH₂CH₂CH₃, C(O)OC(CH₃)₂, or C(O)OH, wherein heterocyclyl is unsubstituted or substituted with methyl or NH₂, and wherein bicyclic heteroaryl is unsubstituted or substituted with ═O, and
R⁵³ is hydrogen, Cl or methyl, or
3) 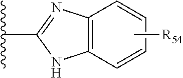
wherein R⁵ is hydrogen or F.

9. A compound of claim 1, or a pharmaceutically acceptable salt thereof, wherein m is 0, n is 0, p is 1, and R⁵ is

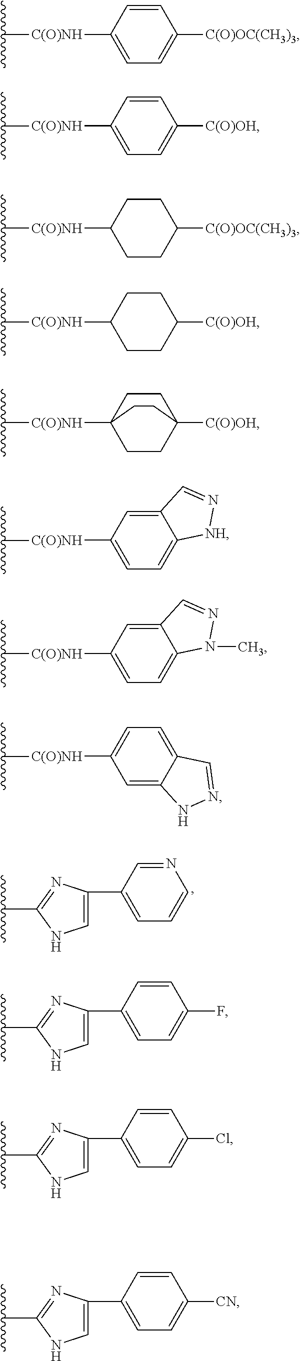

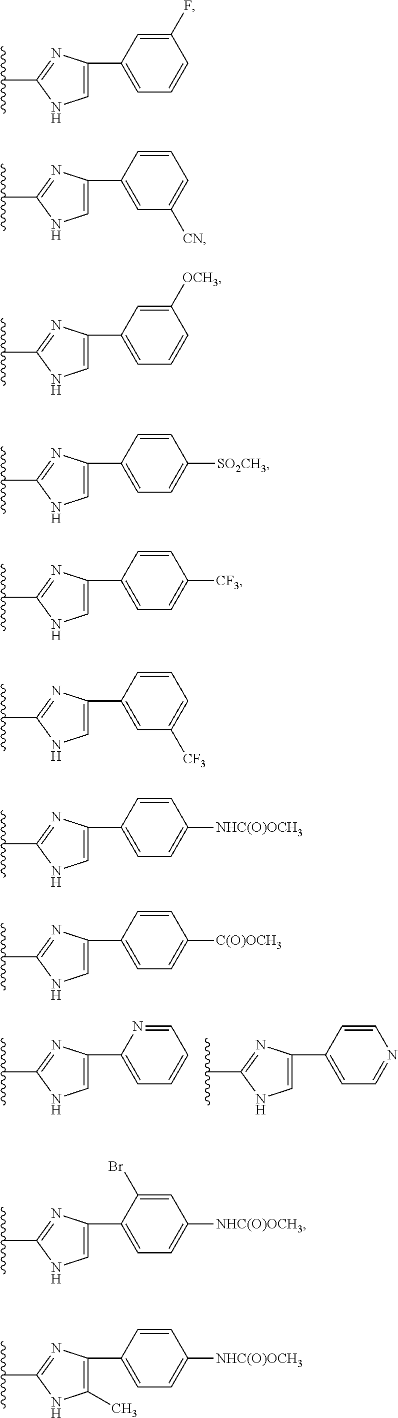

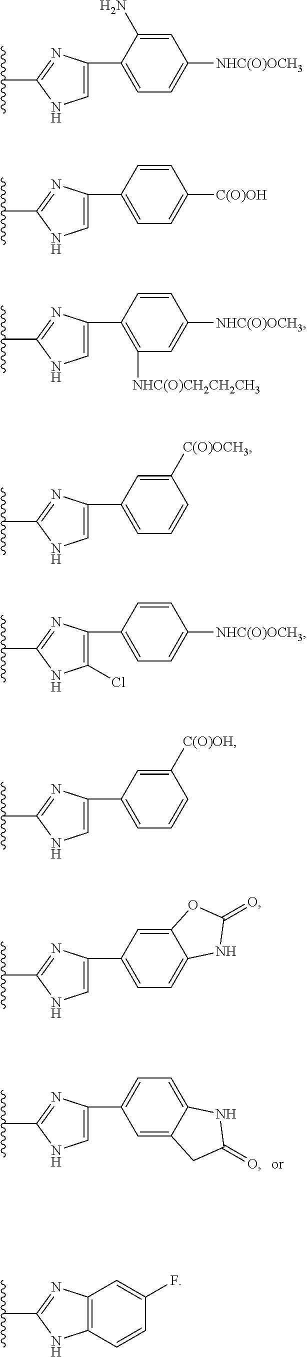

10. A compound of claim 1, or a pharmaceutically acceptable salt thereof, which is
(E)-tert-butyl 4-(1-(3-(5-chloro-2-(1H-tetrazol-1-yl)phenyl)acryloyl)-3-phenylpiperidine-2-carboxamido)benzoate,
(E)-1-(2-(3-(2-((4-carboxyphenyl)carbamoyl)-3-phenylpiperidin-1-yl)-3-oxoprop-1-en-1-yl)-4-chlorophenyl)-1H-tetrazol-2-ium 2,2,2-trifluoroacetate,
(E)-tert-butyl 4-(1-(3-(5-chloro-2-(1H-tetrazol-1-yl)phenyl)acryloyl)-3-cyclohexylpiperidine-2-carboxamido)benzoate,
(E)-tert-butyl 4-(1-(3-(5-chloro-2-(1H-tetrazol-1-yl)phenyl)acryloyl)-3-cyclohexylpiperidine-2-carboxamido)cyclohexanecarboxylate,
(E)-1-(2-(3-(2-((4-carboxyphenyl)carbamoyl)-3-cyclohexylpiperidin-1-yl)-3-oxoprop-1-en-1-yl)-4-chlorophenyl)-1H-tetrazol-2-ium 2,2,2-trifluoroacetate,
1-6: (E)-1-(2-(3-(2-((4-carboxyhexyl)carbamoyl)-3-cyclohexylpiperidin-1-yl)-3-oxoprop-1-en-1-yl)-4-chlorophenyl)-1H-tetrazol-2-ium 2,2,2-trifluoroacetate,
(E)-3-(5-chloro-2-(1H-tetrazol-1-yl)phenyl)-1-(3-phenyl-2-(4-(pyridin-3-yl)-1H-imidazol-2-yl)piperidin-1-yl)prop-2-en-1-one,
4-(1-(1-carbamimidoylpiperidine-4-carbonyl)-3-phenylpiperidine-2-carboxamido)benzoic acid,
4-(1-(3-(5-chloro-2-(1H-tetrazol-1-yl)phenyl)propanoyl)-3-phenylpiperidine-2-carboxamido)benzoic acid,
(E)-4-(1-(3-(5-chloro-2-(1H-tetrazol-1-yl)phenyl)acryloyl)-3-(3-fluorophenyl)piperidine-2-carboxamido)benzoic acid,
(E)-3-(5-chloro-2-(1H-tetrazol-1-yl)phenyl)-1-(3-phenyl-2-(4-(pyridin-3-yl)-1H-imidazol-2-yl)piperidin-1-yl)prop-2-en-1-one,
(E)-3-(5-chloro-2-(1H-tetrazol-1-yl)phenyl)-1-(3-phenyl-2-(4-(pyridin-3-yl)-1H-imidazol-2-yl)piperidin-1-yl)prop-2-en-1-one,
(E)-3-(5-chloro-2-(1H-tetrazol-1-yl)phenyl)-1-(2-(4-(4-fluorophenyl)-1H-imidazol-2-yl)-3-phenylpiperidin-1-yl)prop-2-en-1-one,
4-(1-(1-amino-2,3-dihydro-1H-indene-5-carbonyl)-3-phenylpiperidine-2-carboxamido)benzoic acid,
4-(1-(4-(2-aminopropan-2-yl)benzoyl)-3-phenylpiperidine-2-carboxamido)benzoic acid,
4-(1-((S)-1-amino-2,3-dihydro-1H-indene-5-carbonyl)-3-phenylpiperidine-2-carboxamido)benzoic acid,
ammonium (E)-4-(1-(3-(5-chloro-2-(1H-tetrazol-1-yl)phenyl)acryloyl)-3-(3-(trifluoromethyl)phenyl)piperidine-2-carboxamido)benzoate,
ammonium (E)-4-(1-(3-(5-chloro-2-(1H-tetrazol-1-yl)phenyl)acryloyl)-3-(4-fluorophenyl)piperidine-2-carboxamido)benzoate,
ammonium (E)-4-(1-(3-(5-chloro-2-(1H-tetrazol-1-yl)phenyl)acryloyl)-3-(2,4-difluorophenyl)piperidine-2-carboxamido)cyclohexanecarboxylate,
(4-(2-((4-carboxyphenyl)carbamoyl)-3-phenylpiperidine-1-carbonyl)phenyl)methanaminium 2,2,2-trifluoroacetate,
(E)-3-(5-chloro-2-(1H-tetrazol-1-yl)phenyl)-1-(2-(4-(4-chlorophenyl)-1H-imidazol-2-yl)-3-phenylpiperidin-1-yl)prop-2-en-1-one,
(E)-4-(2-(1-(3-(5-chloro-2-(1H-tetrazol-1-yl)phenyl)acryloyl)-3-phenylpiperidin-2-yl)-1H-imidazol-4-yl)benzonitrile,
3-(5-chloro-2-(1H-tetrazol-1-yl)phenyl)-1-(2-(4-(4-chorophenyl)-1H-imidazol-2-yl)-3-phenylpiperidin-1-yl)propan-1-one,
4-(2-(1-(3-(5-chloro-2-(1H-tetrazol-1-yl)phenyl)propanoyl)-3-phenylpiperidin-2-yl)-1H-imidazol-4-yl)benzonitrile,
(E)-4-(1-(3-(5-chloro-2-(1H-tetrazol-1-yl)phenyl)acryloyl)-3-(3-(trifluoromethyl)phenyl)piperidine-2-carboxamido)benzoic acid,
E)-4-(1-(3-(5-chloro-2-(1H-tetrazol-1-yl)phenyl)acryloyl)-3-(4-fluorophenyl)piperidine-2-carboxamido)benzoic acid,
(E)-4-(1-(3-(5-chloro-2-(1H-tetrazol-1-yl)phenyl)acryloyl)-3-(2,4-difluorophenyl)piperidine-2-carboxamido)benzoic acid,
(E)-4-(1-(3-(5-chloro-2-(1H-tetrazol-1-yl)phenyl)acryloyl)-3-(2,4-difluorophenyl)piperidine-2-carboxamido)benzoic acid,
(E)-3-(5-chloro-2-(1H-tetrazol-1-yl)phenyl)-1-(2-(4-(3-fluorophenyl)-1H-imidazol-2-yl)-3-phenylpiperidin-1-yl)prop-2-en-1-one,
(E)-3-(2-(1-(3-(5-chloro-2-(1H-tetrazol-1-yl)phenyl)acryloyl)-3-phenylpiperidin-2-yl)-1H-imidazol-4-yl)benzonitrile,
(E)-3-(5-chloro-2-(1H-tetrazol-1-yl)phenyl)-1-(2-(4-(3-methoxyphenyl)-1H-imidazol-2-yl)-3-phenylpiperidin-1-yl)prop-2-en-1-one,
(E)-3-(5-chloro-2-(1H-tetrazol-1-yl)phenyl)-1-(2-(4-(4-(methylsulfonyl)phenyl)-1H-imidazol-2-yl)-3-phenylpiperidin-1-yl)prop-2-en-1-one,
(E)-3-(5-chloro-2-(1H-tetrazol-1-yl)phenyl)-1-(3-phenyl-2-(4-(4-(trifluoromethyl)phenyl)-1H-imidazol-2-yl)piperidin-1-yl)prop-2-en-1-one,
(E)-3-(5-chloro-2-(1H-tetrazol-1-yl)phenyl)-1-(3-phenyl-2-(4-(3-(trifluoromethyl)phenyl)-1H-imidazol-2-yl)piperidin-1-yl)prop-2-en-1-one,
(E)-4-(1-(3-(5-chloro-2-(1H-tetrazol-1-yl)phenyl)acryloyl)-3-(4-(trifluoromethyl)phenyl)piperidine-2-carboxamido)benzoic acid,
(E)-4-(3-(4-aminophenyl)-1-(3-(5-chloro-2-(1H-tetrazol-1-yl)phenyl)acryloyl)piperidine-2-carboxamido)benzoic,
4-(3-(4-((E)-3-(5-chloro-2-(1H-tetrazol-1-yl)phenyl)acrylamido)phenyl)-1-((E)-3-(5-chloro-2-(1H-tetrazol-1-yl)phenyl)acryloyl)piperidine-2-carboxamido)benzoic acid,
(E)-4-(1-(3-(5-chloro-2-(1H-tetrazol-1-yl)phenyl)acryloyl)-3-(4-(trifluoromethoxy)phenyl)piperidine-2-carboxamido)benzoic acid,
(E)-4-(3-(4-carbamoylphenyl)-1-(3-(5-chloro-2-(1H-tetrazol-1-yl)phenyl)acryloyl)piperidine-2-carboxamido)benzoic acid,
(E)-4-(1-(3-(5-chloro-2-(1H-tetrazol-1-yl)phenyl)acryloyl)-3-(4-fluoro-3-methylphenyl)piperidine-2-carboxamido)benzoic acid,
(E)-methyl (4-(2-(1-(3-(5-chloro-2-(1H-tetrazol-1-yl)phenyl)acryloyl)-3-phenylpiperidin-2-yl)-1H-imidazol-4-yl)phenyl)carbamate,
(4-(2-((4-carboxyphenyl)carbamoyl)-3-phenylpiperidine-1-carbonyl)cyclohexyl)methanaminium 2,2,2-trifluoroacetate,
(E)-methyl 4-(2-(1-(3-(5-chloro-2-(1H-tetrazol-1-yl)phenyl)acryloyl)-3-phenylpiperidin-2-yl)-1H-imidazol-4-yl)benzoate,
(E)-3-(5-chloro-2-(1H-tetrazol-1-yl)phenyl)-1-(3-phenyl-2-(4-(pyridin-2-yl)-1H-imidazol-2-yl)piperidin-1-yl)prop-2-en-1-one,
(E)-3-(5-chloro-2-(1H-tetrazol-1-yl)phenyl)-1-(3-phenyl-2-(4-(pyridin-4-yl)-1H-imidazol-2-yl)piperidin-1-yl)prop-2-en-1-one,
(4-(2-((4-carboxyphenyl)carbamoyl)-3-phenylpiperidine-1-carbonyl)-2-cyclopropylphenyl)methanaminium 2,2,2-trifluoroacetate,
(E)-methyl (3-bromo-4-(2-(1-(3-(5-chloro-2-(1H-tetrazol-1-yl)phenyl)acryloyl)-3-phenylpiperidin-2-yl)-1H-imidazol-4-yl)phenyl)carbamate,
(E)-4-(2-(1-(3-(5-chloro-2-(1H-tetrazol-1-yl)phenyl)acryloyl)-3-phenylpiperidin-2-yl)-1H-imidazol-4-yl)benzoic acid,
(E)-methyl (4-(2-(1-(3-(5-chloro-2-(1H-tetrazol-1-yl)phenyl)acryloyl)-3-phenylpiperidin-2-yl)-1H-imidazol-4-yl)phenyl)carbamate,
(E)-methyl (4-(2-(1-(3-(5-chloro-2-(1H-tetrazol-1-yl)phenyl)acryloyl)-3-phenylpiperidin-2-yl)-1H-imidazol-4-yl)phenyl)carbamate,
(E)-methyl (3-amino-4-(2-(1-(3-(5-chloro-2-(1H-tetrazol-1-yl)phenyl)acryloyl)-3-phenylpiperidin-2-yl)-1H-imidazol-4-yl)phenyl)carbamate,
4-(1-(4-(aminomethyl)cyclohexanecarbonyl)-3-phenylpiperidine-2-carboxamido)benzoic acid,
4-(1-(4-(aminomethyl)cyclohexanecarbonyl)-3-phenylpiperidine-2-carboxamido)benzoic acid,
(E)-methyl (3-butyramido-4-(2-(1-(3-(5-chloro-2-(1H-tetrazol-1-yl)phenyl)acryloyl)-3-phenylpiperidin-2-yl)-1H-imidazol-4-yl)phenyl)carbamate,
methyl (4-(2-((2S,3S)-1-((E)-3-(5-chloro-2-(1H-tetrazol-1-yl)phenyl)acryloyl)-3-phenylpiperidin-2-yl)-5-methyl-1H-imidazol-4-yl)phenyl)carbamate,
(E)-6-(2-(1-(3-(5-chloro-2-(1H-tetrazol-1-yl)phenyl)acryloyl)-3-phenylpiperidin-2-yl)-1H-imidazol-4-yl)benzo[d]oxazol-2(3H)-one,
(E)-methyl 3-(2-(1-(3-(5-chloro-2-(1H-tetrazol-1-yl)phenyl)acryloyl)-3-phenylpiperidin-2-yl)-1H-imidazol-4-yl)benzoate,
methyl (4-(5-chloro-2-((2S,3S)-1-((E)-3-(5-chloro-2-(1H-tetrazol-1-yl)phenyl)acryloyl)-3-phenylpiperidin-2-yl)-1H-imidazol-4-yl)phenyl)carbamate,
4-((2R,3R)-1-((E)-3-(5-chloro-2-(1H-tetrazol-1-yl)phenyl)acryloyl)-3-phenylpiperidine-2-carboxamido)benzoic acid,
4-((2S,3S)-1-((E)-3-(5-chloro-2-(1H-tetrazol-1-yl)phenyl)acryloyl)-3-phenylpiperidine-2-carboxamido)benzoic acid,
(E)-3-(2-(1-(3-(5-chloro-2-(1H-tetrazol-1-yl)phenyl)acryloyl)-3-phenylpiperidin-2-yl)-1H-imidazol-4-yl)benzoic acid
(E)-1-(3-(5-chloro-2-(1H-tetrazol-1-yl)phenyl)acryloyl)-N-(1H-indazol-5-yl)-3-phenylpiperidine-2-carboxamide,
(E)-1-(3-(5-chloro-2-(1H-tetrazol-1-yl)phenyl)acryloyl)-N-(1H-indazol-5-yl)-3-phenylpiperidine-2-carboxamide,
(E)-1-(3-(5-chloro-2-(1H-tetrazol-1-yl)phenyl)acryloyl)-N-(1H-indazol-6-yl)-3-phenylpiperidine-2-carboxamide,
5-(2-((2R,3R)-1-((E)-3-(5-chloro-2-(1H-tetrazol-1-yl)phenyl)acryloyl)-3-phenylpiperidin-2-yl)-1H-imidazol-5-yl)indolin-2-one
5-(2-((2S,3S)-1-((E)-3-(5-chloro-2-(1H-tetrazol-1-yl)phenyl)acryloyl)-3-phenylpiperidin-2-yl)-1H-imidazol-5-yl)indolin-2-one,
methyl (4-(5-chloro-2-((2R,3R)-1-((E)-3-(5-chloro-2-(1H-tetrazol-1-yl)phenyl)acryloyl)-3-phenylpiperidin-2-yl)-1H-imidazol-4-yl)phenyl)carbamate,
(2S,3S)-1-((E)-3-(5-chloro-2-(1H-tetrazol-1-yl)phenyl)acryloyl)-N-(1H-indazol-5-yl)-3-phenylpiperidine-2-carboxamide,
methyl (4-(2-((2S,3R)-3-(((tert-butyldimethylsilyl)oxy)methyl)-1-((E)-3-(5-chloro-2-(1H-tetrazol-1-yl)phenyl)acryloyl)piperidin-2-yl)-1H-imidazol-4-yl)phenyl)carbamate,
methyl (4-(2-((2S,3R)-1-((E)-3-(5-chloro-2-(1H-tetrazol-1-yl)phenyl)acryloyl)-3-(hydroxymethyl)piperidin-2-yl)-1H-imidazol-4-yl)phenyl)carbamate,
(E)-3-(5-chloro-2-(1H-tetrazol-1-yl)phenyl)-1-((2S,3S)-2-(5-fluoro-1H-benzo[d]imidazol-2-yl)-3-phenylpiperidin-1-yl)prop-2-en-1-one,
3-(2-((2S,3S)-1-((E)-3-(5-chloro-2-(1H-tetrazol-1-yl)phenyl)acryloyl)-3-phenylpiperidin-2-yl)-1H-imidazol-4-yl)benzoic acid,
3-(2-((2R,3R)-1-((E)-3-(5-chloro-2-(1H-tetrazol-1-yl)phenyl)acryloyl)-3-phenylpiperidin-2-yl)-1H-imidazol-4-yl)benzoic acid,
(E)-4-(1-(3-(5-chloro-2-(1H-tetrazol-1-yl)phenyl)acryloyl)-5-phenylpiperidine-2-carboxamido)benzoic acid, or
4-((2S,3S)-1-((E)-3-(5-chloro-2-(1H-tetrazol-1-yl)phenyl)acryloyl)-3-phenylpiperidine-2-carboxamido)bicyclo[2.2.2]octane-1-carboxylic acid.

11. A composition for inhibiting thrombus formation in blood comprising a compound of claim 1 and a pharmaceutically acceptable carrier.

12. A method for inhibiting thrombin in blood comprising adding to the blood a composition of claim 11.

13. A method for inhibiting formation of blood platelet aggregates in blood comprising adding to the blood a composition of claim 11.

14. A method for inhibiting thrombus formation in blood comprising adding to the blood a composition of claim 11.

15. (canceled)

16. A method for treating or preventing venous thromboembolism and pulmonary embolism in a mammal comprising administering to the mammal a composition of claim 11.

17. A method for treating or preventing deep vein thrombosis in a mammal comprising administering to the mammal a composition of claim 11.

18. A method for treating or preventing thromboembolic stroke in humans and other mammals comprising administering to the mammal a composition of claim 11.
